(19) United States
(12) Patent Application Publication
(10) Pub. No.: US 2009/0221648 A1
(43) Pub. Date: Sep. 3, 2009

(54) COMPOSITIONS FOR TREATMENT OF COGNITIVE DISORDERS

(75) Inventors: Lynne E. Rueter, Round Lake Beach, IL (US); Gerald B. Fox, Gurnee, IL (US); Kaitlin E. Browman, Deerfield, IL (US); James P. Sullivan, Deerfield, IL (US); Jorge D. Brioni, Vernon Hills, IL (US); Marlon D. Cowart, Round Lake Beach, IL (US); Ramin Faghih, Lake Forest, IL (US); Gregory A. Gfesser, Lindenhurst, IL (US); Kathleen H. Mortell, Chicago, IL (US); Murali Gopalakrishnan, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/338,865

(22) Filed: Dec. 18, 2008

Related U.S. Application Data
(60) Provisional application No. 61/017,388, filed on Dec. 28, 2007.
(60) Provisional application No. 61/015,996, filed on Dec. 21, 2007.

(51) Int. Cl. A61K 31/465; A61P 25/00

(52) U.S. Cl. 514/343

(57) ABSTRACT

The present invention relates to compositions for treatment of cognitive deficits. Such compositions can have a nicotinic acetylcholine receptor ligand, of either α4β2 subtype or α7 nicotinic acetylcholine subtype, and a histamine-3 receptor ligand, a method of using the same, and a related article of manufacture. Combinations of such active agents with psychostimulants or monoamine reuptake inhibitors also is contemplated.

### CROSS-REFERENCE TO RELATED APPLICATIONS

This application claims priority to U.S. Provisional Patent Application Ser. No. 61/015,996, filed Dec. 21, 2007, and U.S. Provisional Patent Application No. 61/017,388, filed on Dec. 28, 2007, both of which are herein incorporated by reference.

### BACKGROUND OF THE INVENTION

1. Technical Field

The present invention relates to compositions for treatment of cognitive disorders, particularly attention-deficit hyperactivity disorder, methods for using such compositions, and a related article of manufacture.

2. Description of Related Technology

Deficits in attention and response inhibition are apparent across several neurodegenerative and neuropsychiatric disorders for which pharmacotherapy is inadequate. Increased impulsivity and deficits in attention are key symptoms frequently observed across a number of different neuropsychiatric and neurodegenerative diseases. Both symptoms are co-morbid in attention-deficit hyperactivity disorder (ADHD), which is estimated to affect 6 to 10% of school-aged children, and in schizophrenia, where poor functional outcome in the majority of patients is predicted by such cognitive and behavioural deficits. In both ADHD and schizophrenia, these deficits ultimately lead to severe impairment of executive functioning, which remains largely untreated.

Attention-deficit hyperactivity disorder is a psychiatric disorder that can first appear in childhood and can also occur into and throughout adulthood. Symptoms of ADHD can include aspects such as selective and divided attention and distractability. In addition, impulsivity, which has been defined as a failure of response inhibition, also is characteristic. Such impulsivity has been linked to an increased probability of suicide, gambling, drug abuse, and aggression. Indeed schizophrenic patients often exhibit higher levels of impulsivity and demonstrate an increased risk for suicide and substance abuse.

The current standard of treatment for ADHD are psychostimulants, for example amphetamine, methylphenidate, and pemoline. Antidepressants such as desimpramine, which acts to selectively block the reuptake or norepinephrine also are effective in some cases. In addition, monoamine reuptake inhibitors, such as atomoxetine, have been used for treatment of such disorders. While current therapies seem to control activity, there remains a need for addressing cognitive deficits in current therapies. Specifically, while psychostimulants are recognized for their broad efficacy, their use is limited by a significant number of dose-dependent side effects including insomnia, decreased appetite, motor tics, restlessness, increased heart rate or blood pressure and irritability. Indeed, methods used in common practice to limit side effects include decreasing doses in the form of skipping doses in the evening and on school holidays (Reeves and Schwietzer, Expert Opin Pharmacother, 5 (2004): 1313-1320). However, decreasing doses in this fashion leads to significant symptom breakthrough. Therefore, pursuing a dose sparing approach with adjunctive therapy of a novel compound plus a low dose of a stimulant that would retain the full efficacy seen with stimulants but with a lower incidence of stimulant-induced side effects would be a significant improvement to current therapies. Furthermore, while reuptake inhibitors such as atomoxetine show a decreased side effect profile relative to the stimulants, they also show less overall clinical efficacy. Therefore, an adjunctive therapy of a novel compound with a reuptake inhibitor that enhances efficacy to levels seen with stimulants without stimulant-induced side effects would be a significant improvement to current therapies.

Accordingly, it would be beneficial to provide therapy, including compositions and methods of treatment, which address cognitive attention performance and impulsivity. Such compositions and methods would provide a benefit for the treatment of cognitive disorders that is remain unmet by current therapies.

### BRIEF DESCRIPTION OF THE DRAWINGS

FIG. 1 is a graphical representation of the transfer latency of atomoxetine relative to vehicle.

FIG. 2 is a graphical representation of the transfer latency of Compound 1, an alpha7 ligand, relative to vehicle.

### SUMMARY OF THE INVENTION

The present invention relates to compositions for treatment of cognitive deficits. Such compositions can have a nicotinic acetylcholine receptor ligand, of either α4β2 subtype or α7 nicotinic acetylcholine subtype, and a histamine-3 receptor ligand, a method of using the same, and a related article of manufacture. Compositions having a histamine-3 receptor ligand in combination with a psychostimulant, a monoamine reuptake inhibitor, or both a psychostimulant and a monoamine reuptake inhibitor, also are contemplated. The invention further contemplates a composition having a nicotinic acetylcholine receptor ligand of α4β2 subtype and a psychostimulant, a monoamine reuptake inhibitor, or both a psychostimulant and a monoamine reuptake inhibitor.

In one embodiment, the invention relates to a composition comprising (i) a nicotinic acetylcholine receptor ligand; and (ii) a histamine-3 receptor ligand.

In another embodiment, the present invention relates to a method for treating or preventing a cognitive condition in a patient. In the method, the steps include, but are not limited to, (i) administering a nicotinic acetylcholine receptor ligand to a patient; and (ii) administering a histamine-3 receptor ligand to a patient to treat or prevent a psychotic condition.

Yet another embodiment relates to an article of manufacture, having (i) a first pharmaceutical dosage form with a nicotinic acetylcholine receptor ligand (ii) a second pharmaceutical dosage form with at least one histamine-3 receptor ligand; and wherein the article contains first and second pharmaceutical dosage forms.

Yet another embodiment relates to a composition comprising (i) a nicotinic acetylcholine receptor ligand or a histamine-3 receptor ligand; and (ii) a psychostimulant, a monoamine reuptake inhibitor, or both.

In another embodiment, the present invention relates to a method for treating or preventing a cognitive condition in a patient. In the method, the steps include, but are not limited to, (i) administering a nicotinic acetylcholine receptor ligand or a histamine-3 receptor ligand to a patient; and (ii) administering in combination with (i) a psychostimulant, a monoamine reuptake inhibitor, or both a psychostimulant and a monoamine reuptake inhibitor, to a patient to treat or prevent a psychotic condition.

Yet another embodiment relates to an article of manufacture, having (i) a first pharmaceutical dosage form with a nicotinic acetylcholine receptor ligand or a histamine-3 receptor ligand and (ii) a second pharmaceutical dosage form with at least a psychostimulant, a monoamine reuptake inhibitor, or both a psychostimulant and a monoamine reuptake inhibitor; wherein the article contains first and second pharmaceutical dosage forms.

The embodiments of the present invention, how to prepare them, and how to use them are further described herein.

### DETAILED DESCRIPTION OF THE INVENTION

### Definition of Terms

The term “alkyl” means a straight or branched chain hydrocarbon containing from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkylcarbonyl” means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(O)—. Representative examples of alkoxycarbonyl include, but are not limited to, methoxycarbonyl, ethoxycarbonyl, and tert-butoxycarbonyl.

The term “aryl” means a monocyclic or bicyclic aromatic ring system. Representative examples of aryl include, but are not limited to, phenyl and naphthyl. Bicyclic ring systems are also exemplified by phenyl ring system fused to a cycloalkyl ring. The bicyclic cycloalkyl is connected to the parent molecular moiety through any carbon atom contained within the phenyl ring. Representative examples of bicyclic ring systems include, but are not limited to, 1,2,3,4-tetrahydronaphthalenyl, and indanyl. The aryl groups of this invention are substituted with 0, 1, 2, 3, 4, or 5 substituents independently selected from acyl, acyloxy, alkenyl, alkoxy, alkoxyalkoxy, alkoxyalkyl, alkoxycarbonyl, alkoxysulfonyl, alkyl, alkylcarbonyl, alkylsulfonyl, alkynyl, carboxy, cyano, cyanoalkyl, cycloalkyl, formyl, haloalkoxy, haloalkyl, halo, hydroxy, hydroxyalkyl, mercapto, morpholinyl, nitro, thioalkoxy, —NRⁱR^{j}, (NRⁱR^{j})alkyl, (NRⁱR^{j})alkoxy, (NRⁱR^{j})carbonyl, (NRⁱR^{j})sulfonyl, —OCH₂CH═CH₂, —OC₆H₅, and pyridyl wherein Rⁱ and R^{j} are each independently hydrogen, alkoxyalkyl, alkoxycarbonyl, alkyl, alkylcarbonyl, or formyl, or Rⁱ and R^{j} taken together with the nitrogen atom to which they are attached, may form a 4, 5, 6 or 7 membered heterocyclic ring.

The term “arylalkoxy” means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “aryloxy” means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “carboxy” means a —CO₂H group.

The term “cyano” means a —CN group.

The cycloalkyl groups of the present invention are optionally substituted with 1, 2, 3, or 4 substituents selected from the group consisting of acyl, acyloxy, alkenyl, alkoxy, alkoxyalkoxy, alkoxyalkyl, alkoxycarbonyl, alkoxysulfonyl, alkyl, alkylsulfonyl, alkynyl, aryl, carboxy, cyano, formyl, haloalkoxy, haloalkyl, halo, hydroxy, hydroxyalkyl, mercapto, nitro, oxo, thioalkoxy, —NRⁱR^{j}, (NRⁱR^{j})alkyl, (NRⁱR^{j})alkoxy, (NRⁱR^{j})carbonyl, and (NRⁱR^{j})sulfonyl, wherein Rⁱ and R^{j} are each independently hydrogen, alkoxyalkyl, alkoxycarbonyl, alkyl, alkylcarbonyl, or formyl, or Rⁱ and R^{j} taken together with the nitrogen atom to which they are attached, may form a 4, 5, 6 or 7 membered heterocyclic ring.

The term “haloalkoxy” means at least one halogen, as defined herein, appended to the parent molecular moiety through an alkoxy group, as defined herein. Representative examples of haloalkoxy include, but are not limited to, chloromethoxy, 2-fluoroethoxy, difluoromethoxy, trifluoromethoxy, and pentafluoroethoxy.

The term “heteroaryl” means a monocyclic heteroaryl or a bicyclic heteroaryl. The monocyclic heteroaryl is a 5 or 6 membered ring containing at least one heteroatom independently selected from the group consisting of O, N, and S. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, imidazolium, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl, or a monocyclic heteroaryl fused to a monocyclic heterocycle. The bicyclic heteroaryl is connected to the parent molecular moiety through any carbon atom or any substitutable nitrogen atom contained within the bicyclic heteroaryl. Representative examples of bicyclic heteroaryl include, but are not limited to, benzimidazolyl, benzofuranyl, benzothiazolyl, benzothienyl, benzoxadiazolyl, cinnolinyl, dihydro-1-oxo-1H-indenyl, dihydroquinolinyl, dihydroisoquinolinyl, furopyridinyl, indazolyl, indolyl, isoquinolinyl, naphthyridinyl, pyrido[3,2-b]pyrazinyl, pyrido[2,3-b]pyrazinyl, quinolinyl, quinoxalinyl, tetrahydroquinolinyl, thienopyridinyl, and triazolopyridinyl.

The heteroaryl groups of the invention are substituted with 0, 1, 2, 3 or 4 substituents independently selected from acyl, acyloxy, alkenyl, alkoxy, alkoxyalkoxy, alkoxyalkyl, alkoxycarbonyl, alkoxysulfonyl, alkyl, alkylsulfonyl, alkynyl, carboxy, cyano, cycloalkyl, formyl, haloalkoxy, haloalkyl, halo, hydroxy, hydroxyalkyl, mercapto, morpholinyl, nitro, thioalkoxy, —NRⁱR^{j}, (NRⁱR^{j})alkyl, (NRⁱR^{j})alkoxy, (NRⁱR^{j})carbonyl, and (NRⁱR^{j})sulfonyl, wherein Rⁱ and R^{j} are each independently hydrogen, alkoxyalkyl, alkoxycarbonyl, alkyl, alkylcarbonyl, or formyl, or Rⁱ and R^{j} taken together with the nitrogen atom to which they are attached, may form a 4, 5, 6 or 7 membered heterocyclic ring.

The term “heterocycle” or “heterocyclic” means a monocyclic heterocycle or a bicyclic heterocycle. The monocyclic heterocycle is a 3, 4, 5, 6 or 7 membered ring containing at least one heteroatom independently selected from the group consisting of O, N, and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 2,5-dihydro-1H-pyrrolyl, 2,3-dihydrothiazolyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a cycloalkyl, or a monocyclic heterocycle fused to a cycloalkenyl, or a monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodithiolyl, 4H-benzo[d][1,3]dioxinyl, bicyclo[2,2,1]diazaheptanyl, 3,6-diazabicyclo[3.2.0]heptanyl, 2,5-diazabicyclo[2.2.1]heptanyl, 2,3-dihydro-1-benzofuranyl, 2,3-dihydro-1-benzothienyl, 2,3-dihydro-1H-indolyl, hexahydropyrrolo[3,4-c]pyrrolyl, indolinyl, octahydropyrrolo[3,4-c]pyrrolyl, octahydro-1H-pyrrolo[3,4-b]pyridinyl, and 1,2,3,4-tetrahydroquinolinyl; provided that 2,3-dihydrobenzo[b][1,4] dioxinyl and benzo[d][1,3]dioxolyl derivatives are excluded.

The heterocycles of this invention are optionally substituted with 1, 2, 3, or 4 substituents independently selected from the group consisting of acyl, acyloxy, alkenyl, alkoxy, alkoxyalkoxy, alkoxyalkyl, alkoxycarbonyl, alkoxysulfonyl, alkyl, alkylcarbonyl, alkylsulfonyl, alkynyl, benzyl, carboxy, cyano, cycloalkyl, formyl, haloalkoxy, haloalkyl, halo, hydroxy, hydroxyalkyl, mercapto, morpholinyl, nitro, phenyl, pyridinyl, pyrimidinyl, thioalkoxy, —NRⁱR^{j}, (NRⁱR^{j})alkyl, (NRⁱR^{j})alkoxy, (NRⁱR^{j})carbonyl, and (NRⁱR^{j})sulfonyl, wherein Rⁱ and R^{j} are each independently hydrogen, alkoxyalkyl, alkoxycarbonyl, alkyl, alkylcarbonyl, or formyl, or Rⁱ and R^{j} taken together with the nitrogen atom to which they are attached, may form a 4, 5, 6 or 7 membered heterocyclic ring.

### Nicotinic Acetylcholine Subtype α4β2 Receptor Ligand

Nicotinic acetylcholine subtype α4β2 receptor ligands modulate the function by altering the activity of the receptor. Suitable compounds also can be partial agonists that partially block or partially activate the α4β2 receptor or agonists that activate the receptor. Nicotinic acetylcholine receptor α4β2 receptor ligands suitable for the invention can include full agonists or partial agonists. Compounds modulating activity of nicotinic acetylcholine receptor α4β2 subtype are suitable for the invention regardless of the manner in which they interact with the receptor.

One manner for characterizing α4β2 receptor ligands is by a binding assay. [³H]-Cytisine binding values (“Kᵢ Cyt”) of compounds of the invention ranged from about 0.001 nanomolar to greater than 100 micromolar. Preferred compounds for the composition demonstrate binding values of from about 0.001 nanomolar to 10 micromolar. The [³H]-cytisine binding assays have been well reported; however, further details for carrying out the assays can be obtained in International Publication No. WO 99/32480; U.S. Pat. Nos. 5,948,793 and 5,914,328; WO 2004/018607; U.S. Pat. No. 6,809,105; WO 00/71534; and U.S. Pat. No. 6,833,370.

Accordingly, α4β2 receptor ligands suitable for the invention can be compounds of various chemical classes. Particularly, some examples of α4β2 receptor ligands suitable for the invention include, but are not limited to heterocyclic ether derivatives, for example as described in International Publication No. WO 99/32480, published Jul. 1, 1999 and further described and claimed in U.S. Pat. Nos. 5,948,793, issued Sep. 7, 1999, and 5,914,328, issued Jun. 22, 1999; N-substituted diazabicyclic derivatives, for example as described in International Publication No. WO 2004/0186107, published Sep. 23, 2004, and further described and claimed in U.S. Pat. No. 6,809,105, issued Oct. 26, 2004; heterocyclic substituted amino azacycles, for example as described in International Publication No. WO 00/71534, published Nov. 30, 2000, and further described and claimed in U.S. Pat. No. 6,833,370, issued Dec. 21, 2004; all of which are hereby incorporated by reference in their entirety. Further description and methods for preparing the compounds have been reported in the patents, patent publications, and international patent publications cited.

Additional examples of α4β2 receptor ligands suitable for the invention include, but are not limited to aryl-fused azapolycyclic compounds, for example as described in International Publication No. WO 2001062736, published Aug. 30, 2001; aryl-substituted olefinic amine compounds, for example as described in International Publication Nos. WO 9965876, published Dec. 23, 1999, and WO 00/75110, published Dec. 14, 2000; pyridopyranoazepine derivatives, for example as described in U.S. Pat. No. 6,538,003, published Mar. 25, 2003; benzylidene- and cinnamylidene-anabaseines, for examples as described in International Publication No. WO 99/10338, published Mar. 4, 1999; and 3-pyridoxylalkyl heterocyclic ether compounds, for example as described in International Publication No. WO 96/040682, published Dec. 19, 1996; all of which are hereby incorporated by reference in their entirety. Further description and methods for preparing the compounds have been reported in the patents and international patent publications cited.

Other compounds reported as demonstrating α4β2 ligands include, but are not limited to, TC-1734 (ispronicline), GTS-21,4-hydroxy-GTS-21, TC-5619, TC-2696, dianicline and varenicline, which are all described in the publicly available literature.

Specific examples of compounds contemplated for the α4β2 receptor ligands include, but are not limited to,
5-[(2R)-azetidin-2-ylmethoxy]-2-chloropyridine;
(3R)-1-pyridin-3-ylpyrrolidin-3-amine;
2-methyl-3-(2-(S)-pyrrolidinylmethoxy)pyridine;
3-(5,6-dichloro-pyridin-3-yl)-1S,5S-3,6-diazabicyclo[3.2.0]heptane;
(R,R)-1-(pyridin-3-yl)octahydro-pyrrolo[3,4-b]pyrrole;
6,10-methano-6H-pyrazino[2,3-h][3]benzazepine;
7,8,9,10-tetrahydro-(2S,4E)-N-methyl-5-(5-isopropoxy-3-pyridyl)-4-penten-2-amine;
(2S,4E)-N-methyl-5-(5-methoxy-3-pyridyl)-4-penten-2-amine;
(2S,4E)-N-methyl-5-(5-ethoxy-3-pyridyl)-4-penten-2-amine;
(2S,4E)-N-methyl-3-pyrimidine-4-penten-2-amine;
(5aS,8S,10aR)-5a,6,9,10-tetrahydro-7H,11H-8,10a-methanopyrido[2′,3′:5,6]pyrano[2,3-d]azepine;
3-[1-(2,4-dimethoxy-phenyl)-meth-(E)-ylidene]-3,4,5,6-tetrahydro-[2,3′]bipyridinyl; and
3-[1-(2-methoxy-4-hydroxyphenyl)-meth-(E)-ylidene]-3,4,5,6-tetrahydro-[2,3′]bipyridinyl;
   or pharmaceutically acceptable salts thereof.
   Nicotinic Acetylcholine Subtype α7 Receptor Ligand Nicotinic acetylcholine subtype α7 receptor ligands modulate the function of nicotinic acetylcholine subtype α7 receptors by altering the activity of the receptor. Suitable compounds also can be partial agonists that partially block or partially activate the α7 receptor or agonists that activate the receptor. Positive allosteric modulators are compounds that potentiate the receptor response to acetylcholine without themselves triggering receptor activation or desensitization, or either, of the receptor. Nicotinic acetylcholine subtype α7 receptor ligands suitable for the invention can include full agonists, partial agonists, or positive allosteric modulators.

One manner to characterize α7 receptor ligands is that they demonstrate Kᵢ values from about 0.1 nanomolar to about 10 micromolar when tested by the [³H]-MLA assay, many having a binding value (“Kᵢ MLA”) of less than 1 micromolar. [³H]-Cytisine binding values (“Kᵢ Cyt”) of compounds of the invention ranged from about 50 nanomolar to greater than 100 micromolar. The determination of preferred compounds typically considered the Kᵢ MLA value as measured by MLA assay in view of the Kᵢ Cyt value as measured by [³H]-cytisine binding, such that in the formula D=Kᵢ Cyt/Kᵢ MLA, D is at least 50. For example, preferred compounds typically exhibit greater potency at α7 receptors compared to α4β2 receptors. Although the MLA and [³H]-cytisine binding assays are well known, further details for carrying out the assays can be obtained in International Publication Nos. WO 2005/028477; WO 2005/066168; WO 2005/066166; WO 2005/066167; WO 2005/077899 and WO 2008/058096, and US Patent Publication Nos. US 20050137184; US20050137204; US20050245531.

Positive allosteric modulators, at concentrations ranging from 1 nM to 10 μM, enhance responses of acetylcholine at α7 nicotinic receptors expressed endogenously in neurons or cell lines, or via expression of recombinant protein in Xenopus oocytes or in cell lines.

Accordingly, α7 receptor ligands suitable for the invention can be compounds of various chemical classes. Particularly, some examples of α7 receptor ligands suitable for the invention include, but are not limited to diazabicycloalkane derivatives, for example as described in International Publication No. WO 2005/028477; spirocyclic quinuclidinic ether derivatives, for example as described in International Publication No. WO 2005/066168; fused bicycloheterocycle substituted quinuclidine derivatives, for example as described in US Publication Nos. US20050137184; US20050137204; and US2005024553 1; 3-quinuclidinyl amino-substituted biaryl derivatives, for example as described in International Publication No. WO 2005/066166; 3-quinuclidinyl heteroatom-bridged biaryl derivatives, for example as described in International Publication No. WO 2005/066167; and amino-substituted tricyclic derivatives, for example as described in International Publication No. WO 2005/077899, all of which are hereby incorporated by reference in their entirety. Although it is described that the use of such α7 receptor ligands can be used in combination with antipsychotics for their cognitive benefits, the use of α7 receptor ligands for improving the efficacy of antipsychotics without exaggerating the side effect profile of such agents apparently is not contemplated.

Suitable methods for preparing diazabicycloalkane derivatives can be found in International Publication WO 2005/028477, published Mar. 31, 2005, which is hereby incorporated by reference in its entirety.

Suitable methods for preparing fused bicycloheterocycle substituted quinuclidine derivatives can be found in US Publication Nos. US20050137184, published on Jun. 23, 2005; US20050137204, published on Jun. 23, 2005; and US20050245531, published on Nov. 3, 2005, each of which is hereby incorporated by reference in its entirety.

Spirocyclic quinuclidinic ether derivatives; 3-quinuclidinyl amino-substituted biaryl derivatives, 3-quinuclidinyl heteroatom-bridged biaryl derivatives; and amino-substituted tricyclic derivatives also can be prepared and are suitable for the present invention. Further description for preparing such compounds can be found in International Publication Nos. WO 2005/066168, published on Jul. 21, 2005; WO 2005/066166, published on Jul. 21, 2005; WO 2005/066167, published on Jul. 21, 2005; and WO 2005/077899, published on Aug. 25, 2005, each of which is hereby incorporated by reference in its entirety.

Examples of compounds reported as α7 agonists or partial agonists are quinuclidine derivatives, for example as described in WO 2004/016608 and WO 2004/022556; and tilorone derivatives, for example also as described in WO 2004/016608.

Specific examples of compounds that are suitable nicotinic subtype α7 receptor ligands include, but are not limited to:
5-(6-[(3R)-1-azabicyclo[2.2.2]oct-3-yloxy]pyridazin-3-yl)-1H-indole;
2-(6-phenylpyridazine-3-yl)octahydropyrrolo[3,4-c]pyrrole;
5-[5-{(1R,5R)-6-methyl-3,6-diaza-bicyclo[3.2.0]hept-3-yl}-pyridin-2-yl]-1H-indole;
5-[6-(cis-5-methyl-hexahydro-pyrrolo[3,4-c]pyrrol-2-yl)-pyridazin-3-yl-1H-indole; and
(1R,4R,5S)-4-(5-phenyl-[1,3,4]thiazdiazol-2-yloxy)-1-aza-tricyclo[3.3.1.1^{3,7}]decane;
   or pharmaceutically acceptable salts thereof.

Example of suitable nicotinic subtype α7 positive allosteric modulcators are compounds of formula (I):

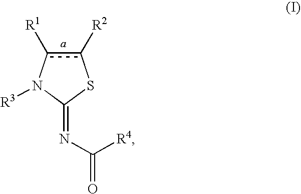

wherein

R¹ is hydrogen, methyl, phenyl, pyrazolyl, or hydroxyl;

R² is hydrogen, alkyl, alkenyl, ═CH₂, or ═CHR^{c} wherein the alkyl group and the alkenyl group are substituted with 0, 1, 2, or 3 substituents independently selected from the group consisting of alkoxycarbonyl, alkylcarbonyloxy, aryl, aryloxy, arylalkoxy, carboxy, cyano, cycloalkyl, haloalkoxy, heteroaryl, heterocycle, hydroxyl, nitro, and R^{d}R^{e}N—, wherein a group represented by R² can be further substituted with 0, 1, or 2 groups selected from halo and alkoxy;

R^{c} is alkyl or halo;

R^{d} and R^{e} are each independently hydrogen, alkyl, alkoxyalkyl, aryl, arylalkyl, cyanoalkyl, heteroaryl, heteroarylalkyl, heterocycle, or heterocyclealkyl;

R³ is optionally substituted aryl, heteroaryl, or -G¹-L-G²;

G¹ is aryl or heteroaryl;

G² is aryl, cycloalkyl, heteroaryl, or heterocycle;

L is a bond, O, alkylene, or —O-alkylene-;

R⁴ is optionally substituted alkyl, cycloalkyl, heteroaryl, heterocycle or —NR⁵R⁶; wherein, R⁵ and R⁶ are independently hydrogen, alkyl, (NRⁱR^{j})alkyl, alkynyl, aryl, heterocyclealkyl, cycloalkylalkyl, cyanoalkyl, cycloalkyl, alkoxyalkyl, arylalkyl, or heteroarylalkyl; and

a is single or double bond;

provided that when R¹ is hydroxyl or when R² is a radical attached to the thiazole ring through an exocyclic double bond, then a is single bond; and

provided that when a is double bond, R¹ is hydrogen, R² is methyl, R³ is phenyl substituted with 3-haloalkyl, 3-halo, 3-haloalkoxy, 2,5-dihalo, 2,3-dihalo, 3,4-dihalo, or 3,5-dihalo, then R⁴ is heterocycle;

or a pharmaceutically acceptable salt, ester, amide or prodrug thereof. Compounds of formula (I) that demonstrate nicotinic subtype α7 positive allosteric modulator activity include, but are not limited to:
N′-[(2Z)-3-(4-methoxyphenyl)-5-methyl-1,3-thiazol-2(3H)-ylidene]-N,N-dimethylurea;
N-[(2Z)-3-[4-(difluoromethoxy)phenyl]-5-methyl-1,3-thiazol-2(3H)-ylidene]pyrrolidine-1-carboxamide;
N-[(2Z)-3-[4-(difluoromethoxy)phenyl]-5-methyl-1,3-thiazol-2(3H)-ylidene]piperidine-1-carboxamide;
N′-[(2Z)-3-[4-(difluoromethoxy)phenyl]-5-methyl-1,3-thiazol-2(3H)-ylidene]-N,N-diethylurea;
N-[(2Z)-5-methyl-3-[4-(trifluoromethoxy)phenyl]-1,3-thiazol-2(3H)-ylidene]pyrrolidine-1-carboxamide;
N,N-diethyl-N′-[(2Z)-5-methyl-3-[4-(trifluoromethoxy)phenyl]-1,3-thiazol-2(3H)-ylidene]urea;
N,N-dimethyl-N′-[(2Z)-5-methyl-3-[4-(trifluoromethoxy)phenyl]-1,3-thiazol-2(3H)-ylidene]urea;
N-[(2Z)-3-(4-chlorophenyl)-5-methyl-1,3-thiazol-2(3H)-ylidene]pyrrolidine-1-carboxamide;
N-[(2Z)-3-(4-chlorophenyl)-5-methyl-1,3-thiazol-2(3H)-ylidene]piperidine-1-carboxamide;
N′-[(2Z)-3-(4-chlorophenyl)-5-methyl-1,3-thiazol-2(3H)-ylidene]-N,N-diethylurea;
N′-[(2Z)-3-(4-chlorophenyl)-5-methyl-1,3-thiazol-2(3H)-ylidene]-N-methyl-N-phenylurea;
N′-[(2Z)-3-(4-chlorophenyl)-5-methyl-1,3-thiazol-2(3H)-ylidene]-N,N-dimethylurea;
N-[(2Z)-3-(4-fluorophenyl)-5-methyl-1,3-thiazol-2(3H)-ylidene]pyrrolidine-1-carboxamide;
N-[(2Z)-3-(4-fluorophenyl)-5-methyl-1,3-thiazol-2(3H)-ylidene]piperidine-1-carboxamide;
N,N-diethyl-N′-[(2Z)-3-(4-fluorophenyl)-5-methyl-1,3-thiazol-2(3H)-ylidene]urea;
N′-[(2Z)-3-(4-fluorophenyl)-5-methyl-1,3-thiazol-2(3H)-ylidene]-N,N-dimethylurea;
N-[(2Z)-3-(4-fluorophenyl)-5-methyl-1,3-thiazol-2(3H)-ylidene]cyclobutanecarboxamide;
N-[(2Z)-5-methyl-3-[4-(trifluoromethyl)phenyl]-1,3-thiazol-2(3H)-ylidene]pyrrolidine-1-carboxamide;
N,N-dimethyl-N′-[(2Z)-5-methyl-3-[4-(trifluoromethyl)phenyl]-1,3-thiazol-2(3H)-ylidene]urea;
2-methyl-N-[(2Z)-5-methyl-3-[4-(trifluoromethyl)phenyl]-1,3-thiazol-2(3H)-ylidene]pyrrolidine-1-carboxamide;
(2R)-2-methyl-N-[(2Z)-5-methyl-3-[4-(trifluoromethyl)phenyl]-1,3-thiazol-2(3H)-ylidene]pyrrolidine-1-carboxamide;
(2S)-2-methyl-N-[(2Z)-5-methyl-3-[4-(trifluoromethyl)phenyl]-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]-2,5-dihydro-1H-pyrrole-1-carboxamide;
N-[(2Z)-3-(4-chlorophenyl)-1,3-thiazolidin-2-ylidene]pyrrolidine-1-carboxamide;
(3R)—N-[(2Z)-3-(4-chlorophenyl)-1,3-thiazolidin-2-ylidene]-3-fluoropyrrolidine-1-carboxamide;
N′-[(2Z)-3-(4-chlorophenyl)-1,3-thiazolidin-2-ylidene]-N-methyl-N-prop-2-ynylurea;
N′-[(2Z)-3-(4-chlorophenyl)-1,3-thiazolidin-2-ylidene]-N-(3-fluorobenzyl)-N-methylurea;
N-{(2Z)-5-methylene-3-[4-(trifluoromethyl)phenyl]-1,3-thiazolidin-2-ylidene}azetidine-1-carboxamide;
N-[(2Z)-5-methyl-3-[4-(trifluoromethyl)phenyl]-1,3-thiazol-2(3H)-ylidene]azetidine-1-carboxamide;
N-[(2Z)-5-methyl-3-(2-naphthyl)-1,3-thiazol-2(3H)-ylidene]pyrrolidine-1-carboxamide;
N-[(2Z)-5-methyl-3-(3-methylisothiazol-5-yl)-1,3-thiazol-2(3H)-ylidene]pyrrolidine-1-carboxamide;
N-[(2Z)-3-(4-bromophenyl)-1,3-thiazol-2(3H)-ylidene]pyrrolidine-1-carboxamide;
N-(3-fluorobenzyl)-N-methyl-N′-[(2Z)-5-methyl-3-(2-naphthyl)-1,3-thiazol-2(3H)-ylidene]urea;
(3R)—N-[(2Z)-3-(5-cyanothien-2-yl)-5-methyl-1,3-thiazol-2(3H)-ylidene]-3-fluoropyrrolidine-1-carboxamide;
N-[(2Z)-5-(hydroxymethyl)-3-(2-naphthyl)-1,3-thiazol-2(3H)-ylidene]pyrrolidine-1-carboxamide;
(3R)-3-fluoro-N-[(2Z)-5-(hydroxymethyl)-3-(2-naphthyl)-1,3-thiazol-2(3H)-ylidene]pyrrolidine-1-carboxamide;
(3R)-3-fluoro-N-[(2Z)-5-(hydroxymethyl)-3-[4-(trifluoromethyl)phenyl]-1,3-thiazol-2(3H)-ylidene]pyrrolidine-1-carboxamide;
(3R)-3-fluoro-N-[(2Z)-5-(hydroxymethyl)-3-[5-(trifluoromethyl)thien-2-yl]-1,3-thiazol-2(3H)-ylidene]pyrrolidine-1-carboxamide;
(3R)—N-[(2Z)-3-(5-chlorothien-2-yl)-5-(hydroxymethyl)-1,3-thiazol-2(3H)-ylidene]-3-fluoropyrrolidine-1-carboxamide;
(3R)—N-[(2Z)-3-(1-benzothien-5-yl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-(2,2,4,4-tetrafluoro-4H-1,3-benzodioxin-6-yl)-1,3-thiazol-2(3H)-ylidene]pyrrolidine-1-carboxamide;
(3R)—N-[(2Z)-3-(5,6-difluoro-2-naphthyl)-5-(hydroxymethyl)-1,3-thiazol-2(3H)-ylidene]-3-fluoropyrrolidine-1-carboxamide;
N-[(2Z)-5-methyl-3-phenyl-1,3-thiazol-2(3H)-ylidene]pyrrolidine-1-carboxamide;
N-[(2Z)-5-methyl-3-(3-methylphenyl)-1,3-thiazol-2(3H)-ylidene]pyrrolidine-1-carboxamide;
N-[(2Z)-5-methyl-3-(4-methylphenyl)-1,3-thiazol-2(3H)-ylidene]pyrrolidine-1-carboxamide;
N-[(2Z)-3-(3-aminophenyl)-5-methyl-1,3-thiazol-2(3H)-ylidene]pyrrolidine-1-carboxamide;
N-[(2Z)-3-(3-hydroxyphenyl)-5-methyl-1,3-thiazol-2(3H)-ylidene]pyrrolidine-1-carboxamide;
N-[(2Z)-3-(4-methoxyphenyl)-5-methyl-1,3-thiazol-2(3H)-ylidene]pyrrolidine-1-carboxamide;
N-[(2Z)-3-(3-fluorophenyl)-5-methyl-1,3-thiazol-2(3H)-ylidene]pyrrolidine-1-carboxamide;
N-[(2Z)-3-(3-chlorophenyl)-5-methyl-1,3-thiazol-2(3H)-ylidene]pyrrolidine-1-carboxamide;
N-[(2Z)-3-(2-bromophenyl)-5-methyl-1,3-thiazol-2(3H)-ylidene]pyrrolidine-1-carboxamide;
N-[(2Z)-3-(3-bromophenyl)-5-methyl-1,3-thiazol-2(3H)-ylidene]pyrrolidine-1-carboxamide;
N-[(2Z)-3-(3-cyanophenyl)-5-methyl-1,3-thiazol-2(3H)-ylidene]pyrrolidine-1-carboxamide;
N-[(2Z)-3-(4-cyanophenyl)-5-methyl-1,3-thiazol-2(3H)-ylidene]pyrrolidine-1-carboxamide;
N-[(2Z)-3-[4-(dimethylamino)phenyl]-5-methyl-1,3-thiazol-2(3H)-ylidene]pyrrolidine-1-carboxamide;
N-[(2Z)-5-methyl-3-[3-(trifluoromethyl)phenyl]-1,3-thiazol-2(3H)-ylidene]pyrrolidine-1-carboxamide;
N-[(2Z)-5-methyl-3-[3-(trifluoromethoxy)phenyl]-1,3-thiazol-2(3H)-ylidene]pyrrolidine-1-carboxamide;
N-[(2Z)-5-methyl-3-(4-phenoxyphenyl)-1,3-thiazol-2(3H)-ylidene]pyrrolidine-1-carboxamide;
N-[(2Z)-3-[4-(benzyloxy)phenyl]-5-methyl-1,3-thiazol-2(3H)-ylidene]pyrrolidine-1-carboxamide;
N-[(2Z)-3-(3,4-dimethylphenyl)-5-methyl-1,3-thiazol-2(3H)-ylidene]pyrrolidine-1-carboxamide;
N-[(2Z)-3-(3,5-dimethylphenyl)-5-methyl-1,3-thiazol-2(3H)-ylidene]pyrrolidine-1-carboxamide;
N-[(2Z)-5-methyl-3-pyridin-2-yl-1,3-thiazol-2(3H)-ylidene]pyrrolidine-1-carboxamide;
N-[(2Z)-3-(3,4-dichlorophenyl)-5-methyl-1,3-thiazol-2(3H)-ylidene]pyrrolidine-1-carboxamide;
N-[(2Z)-3-(3,5-dichlorophenyl)-5-methyl-1,3-thiazol-2(3H)-ylidene]pyrrolidine-1-carboxamide;
N-[(2Z)-5-methyl-3-[4-(methylthio)phenyl]-1,3-thiazol-2(3H)-ylidene]pyrrolidine-1-carboxamide;
N-[(2Z)-3-(3-methoxyphenyl)-5-methyl-1,3-thiazol-2(3H)-ylidene]pyrrolidine-1-carboxamide;
N-[(2Z)-3-(4-chloro-3-methylphenyl)-5-methyl-1,3-thiazol-2(3H)-ylidene]pyrrolidine-1-carboxamide;
N-[(2Z)-3-(4-benzylphenyl)-5-methyl-1,3-thiazol-2(3H)-ylidene]pyrrolidine-1-carboxamide;
N-[(2Z)-3-(1-benzothien-5-yl)-5-methyl-1,3-thiazol-2(3H)-ylidene]pyrrolidine-1-carboxamide;
N-[(2Z)-5-methyl-3-(5-methylthien-2-yl)-1,3-thiazol-2(3H)-ylidene]pyrrolidine-1-carboxamide;
N-[(2Z)-5-methyl-3-(2-methyl-1,3-benzothiazol-5-yl)-1,3-thiazol-2(3H)-ylidene]pyrrolidine-1-carboxamide;
N-[(2Z)-5-methyl-3-[6-(trifluoromethyl)pyridin-3-yl]-1,3-thiazol-2(3H)-ylidene]pyrrolidine-1-carboxamide;
N-[(2Z)-5-methyl-3-[5-(trifluoromethyl)pyridin-3-yl]-1,3-thiazol-2(3H)-ylidene]pyrrolidine-1-carboxamide;
N-[(2Z)-3-[4-(methylthio)phenyl]-1,3-thiazol-2(3H)-ylidene]pyrrolidine-1-carboxamide;
N-[(2Z)-3-phenyl-1,3-thiazol-2(3H)-ylidene]pyrrolidine-1-carboxamide;
N-[(2Z)-3-(3-chlorophenyl)-1,3-thiazol-2(3H)-ylidene]pyrrolidine-1-carboxamide;
N-[(2Z)-3-[3-(trifluoromethyl)phenyl]-1,3-thiazol-2(3H)-ylidene]pyrrolidine-1-carboxamide;
N-[(2Z)-3-(4-chlorophenyl)-1,3-thiazol-2(3H)-ylidene]pyrrolidine-1-carboxamide;
N-[(2Z)-3-(4-methylphenyl)-1,3-thiazol-2(3H)-ylidene]pyrrolidine-1-carboxamide;
N-[(2Z)-3-(4-chloro-3-methylphenyl)-1,3-thiazol-2(3H)-ylidene]pyrrolidine-1-carboxamide;
N-[(2Z)-3-(3,4-dichlorophenyl)-1,3-thiazol-2(3H)-ylidene]pyrrolidine-1-carboxamide;
N-[(2Z)-3-(3,4-dimethylphenyl)-1,3-thiazol-2(3H)-ylidene]pyrrolidine-1-carboxamide;
N-[(2Z)-3-(4-cyanophenyl)-1,3-thiazol-2(3H)-ylidene]pyrrolidine-1-carboxamide;
N-[(2Z)-3-(3-chloro-4-methylphenyl)-1,3-thiazol-2(3H)-ylidene]pyrrolidine-1-carboxamide;
N-[(2Z)-3-[4-(difluoromethoxy)phenyl]-1,3-thiazol-2(3H)-ylidene]pyrrolidine-1-carboxamide;
N-[(2Z)-3-(4-fluorophenyl)-5-methyl-1,3-thiazol-2(3H)-ylidene]-2,2-dimethylpropanamide;
N-[(2Z)-3-(4-chlorophenyl)-5-methyl-1,3-thiazol-2(3H)-ylidene]-2,2-dimethylpropanamide;
2,2-dimethyl-N-[(2Z)-5-methyl-3-[4-(trifluoromethyl)phenyl]-1,3-thiazol-2(3H)-ylidene]propanamide;
N-[(2Z)-3-[6-(trifluoromethyl)pyridin-3-yl]-1,3-thiazol-2(3H)-ylidene]pyrrolidine-1-carboxamide;
N-[(2Z)-3-[3-fluoro-4-(trifluoromethyl)phenyl]-1,3-thiazol-2(3H)-ylidene]pyrrolidine-1-carboxamide;
N-[(2Z)-3-(6-fluoro-5-methylpyridin-3-yl)-1,3-thiazol-2(3H)-ylidene]pyrrolidine-1-carboxamide;
2,2-dimethyl-N-[(2Z)-5-methyl-3-(5-methylthien-2-yl)-1,3-thiazol-2(3H)-ylidene]propanamide;
N-[(2Z)-3-(6-fluoropyridin-3-yl)-5-methyl-1,3-thiazol-2(3H)-ylidene]-2,2-dimethylpropanamide;
2,2-dimethyl-N-[(2Z)-5-methyl-3-[6-(trifluoromethyl)pyridin-3-yl]-1,3-thiazol-2(3H)-ylidene]propanamide;
N-[(2Z)-3-[3-fluoro-4-(trifluoromethyl)phenyl]-5-methyl-1,3-thiazol-2(3H)-ylidene]-2,2-dimethylpropanamide;
N-[(2Z)-3-(6-fluoro-5-methylpyridin-3-yl)-5-methyl-1,3-thiazol-2(3H)-ylidene]-2,2-dimethylpropanamide;
2,2-dimethyl-N-[(2Z)-5-methyl-3-[6-(1H-pyrazol-1-yl)pyridin-2-yl]-1,3-thiazol-2(3H)-ylidene]propanamide;
N-[(2Z)-3-(2-fluoropyridin-4-yl)-5-methyl-1,3-thiazol-2(3H)-ylidene]-2,2-dimethylpropanamide;
(3R)—N-[(2Z)-3-(3-bromophenyl)-5-methyl-1,3-thiazol-2(3H)-ylidene]-3-fluoropyrrolidine-1-carboxamide;
(3R)—N-[(2Z)-3-(4-cyano-3-methylphenyl)-5-methyl-1,3-thiazol-2(3H)-ylidene]-3-fluoropyrrolidine-1-carboxamide;
(3R)-3-fluoro-N-[(2Z)-5-methyl-3-(3-methylphenyl)-1,3-thiazol-2(3H)-ylidene]pyrrolidine-1-carboxamide;
(3R)—N-[(2Z)-3-(4-bromophenyl)-5-methyl-1,3-thiazol-2(3H)-ylidene]-3-fluoropyrrolidine-1-carboxamide;
(3R)-3-fluoro-N-[(2Z)-5-methyl-3-(4-phenoxyphenyl)-1,3-thiazol-2(3H)-ylidene]pyrrolidine-1-carboxamide;
(3R)-3-fluoro-N-[(2Z)-5-methyl-3-(4-methylphenyl)-1,3-thiazol-2(3H)-ylidene]pyrrolidine-1-carboxamide;
(3R)—N-[(2Z)-3-(4-ethylphenyl)-5-methyl-1,3-thiazol-2(3H)-ylidene]-3-fluoropyrrolidine-1-carboxamide;
(3R)-3-fluoro-N-[(2Z)-3-(3-fluorophenyl)-5-methyl-1,3-thiazol-2(3H)-ylidene]pyrrolidine-1-carboxamide;
(3R)-3-fluoro-N-[(2Z)-3-(4-fluorophenyl)-5-methyl-1,3-thiazol-2(3H)-ylidene]pyrrolidine-1-carboxamide;
(3R)-3-fluoro-N-[(2Z)-5-methyl-3-[3-(trifluoromethyl)phenyl]-1,3-thiazol-2(3H)-ylidene]pyrrolidine-1-carboxamide;
(3R)-3-fluoro-N-[(2Z)-5-methyl-3-[4-(trifluoromethyl)phenyl]-1,3-thiazol-2(3H)-ylidene]pyrrolidine-1-carboxamide;
(3R)—N-[(2Z)-3-(4-chlorophenyl)-5-methyl-1,3-thiazol-2(3H)-ylidene]-3-fluoropyrrolidine-1-carboxamide;
(3R)-3-fluoro-N-[(2Z)-3-(4-iodophenyl)-5-methyl-1,3-thiazol-2(3H)-ylidene]pyrrolidine-1-carboxamide;
(3R)—N-[(2Z)-3-(3-cyanophenyl)-5-methyl-1,3-thiazol-2(3H)-ylidene]-3-fluoropyrrolidine-1-carboxamide;
(3R)-3-fluoro-N-[(2Z)-5-methyl-3-(2-naphthyl)-1,3-thiazol-2(3H)-ylidene]pyrrolidine-1-carboxamide;
(3R)—N-[(2Z)-3-(5-bromothien-2-yl)-5-methyl-1,3-thiazol-2(3H)-ylidene]-3-fluoropyrrolidine-1-carboxamide;
(3R)—N-[(2Z)-3-(3,4-dichlorophenyl)-5-methyl-1,3-thiazol-2(3H)-ylidene]-3-fluoropyrrolidine-1-carboxamide;
(3R)-3-fluoro-N-[(2Z)-3-(3-iodophenyl)-5-methyl-1,3-thiazol-2(3H)-ylidene]pyrrolidine-1-carboxamide;
(3R)—N-[(2Z)-3-(3-chloro-4-methylphenyl)-5-methyl-1,3-thiazol-2(3H)-ylidene]-3-fluoropyrrolidine-1-carboxamide;
(3R)—N-[(2Z)-3-(1-benzothien-5-yl)-5-methyl-1,3-thiazol-2(3H)-ylidene]-3-fluoropyrrolidine-1-carboxamide;
N-[(2Z)-3-(5-bromothien-2-yl)-1,3-thiazol-2(3H)-ylidene]pyrrolidine-1-carboxamide;
N-[(2Z)-3-(2-naphthyl)-1,3-thiazol-2(3H)-ylidene]pyrrolidine-1-carboxamide;
N-[(2Z)-3-(5-chlorothien-2-yl)-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]indoline-1-carboxamide;
N-ethyl-N-methyl-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-propylurea;
N-(1,3-dioxolan-2-ylmethyl)-N-methyl-N′-[(2Z)-5-methyl-3-[4-(trifluoromethyl)phenyl]-1,3-thiazol-2(3H)-ylidene]urea;
N-methyl-N-(3-methylbutyl)-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-prop-2-ynylurea;
N,N-diethyl-N′-[(2Z)-5-methyl-3-[4-(trifluoromethyl)phenyl]-1,3-thiazol-2(3H)-ylidene]urea;
N-ethyl-N′-[(2Z)-5-methyl-3-[4-(trifluoromethyl)phenyl]-1,3-thiazol-2(3H)-ylidene]-N-propylurea;
N-butyl-N-ethyl-N′-[(2Z)-5-methyl-3-[4-(trifluoromethyl)phenyl]-1,3-thiazol-2(3H)-ylidene]urea;
N,N-dibutyl-N′-[(2Z)-5-methyl-3-[4-(trifluoromethyl)phenyl]-1,3-thiazol-2(3H)-ylidene]urea;
2,5-dimethyl-N-[(2Z)-5-methyl-3-[4-(trifluoromethyl)phenyl]-1,3-thiazol-2(3H)-ylidene]pyrrolidine-1-carboxamide;
2-methyl-N-[(2Z)-5-methyl-3-[4-(trifluoromethyl)phenyl]-1,3-thiazol-2(3H)-ylidene]piperidine-1-carboxamide;
N-(2-methoxyethyl)-N-methyl-N′-[(2Z)-5-methyl-3-[4-(trifluoromethyl)phenyl]-1,3-thiazol-2(3H)-ylidene]urea;
N-benzyl-N-ethyl-N′-[(2Z)-5-methyl-3-[4-(trifluoromethyl)phenyl]-1,3-thiazol-2(3H)-ylidene]urea;
N-benzyl-N-isopropyl-N′-[(2Z)-5-methyl-3-[4-(trifluoromethyl)phenyl]-1,3-thiazol-2(3H)-ylidene]urea;
N-benzyl-N-butyl-N′-[(2Z)-5-methyl-3-[4-(trifluoromethyl)phenyl]-1,3-thiazol-2(3H)-ylidene]urea;
4-benzyl-N-[(2Z)-5-methyl-3-[4-(trifluoromethyl)phenyl]-1,3-thiazol-2(3H)-ylidene]piperidine-1-carboxamide;
N-isopropyl-N′-[(2Z)-5-methyl-3-[4-(trifluoromethyl)phenyl]-1,3-thiazol-2(3H)-ylidene]urea;
N-(sec-butyl)-N′-[(2Z)-5-methyl-3-[4-(trifluoromethyl)phenyl]-1,3-thiazol-2(3H)-ylidene]urea;
N-(1-methylbutyl)-N′-[(2Z)-5-methyl-3-[4-(trifluoromethyl)phenyl]-1,3-thiazol-2(3H)-ylidene]urea;
N-(1,1-dimethylpropyl)-N′-[(2Z)-5-methyl-3-[4-(trifluoromethyl)phenyl]-1,3-thiazol-2(3H)-ylidene]urea;
N-(1-ethylpropyl)-N′-[(2Z)-5-methyl-3-[4-(trifluoromethyl)phenyl]-1,3-thiazol-2(3H)-ylidene]urea;
N-(2-methoxy-1-methylethyl)-N′-[(2Z)-5-methyl-3-[4-(trifluoromethyl)phenyl]-1,3-thiazol-2(3H)-ylidene]urea;
N-cyclopropyl-N′-[(2Z)-5-methyl-3-[4-(trifluoromethyl)phenyl]-1,3-thiazol-2(3H)-ylidene]urea;
N-cyclobutyl-N′-[(2Z)-5-methyl-3-[4-(trifluoromethyl)phenyl]-1,3-thiazol-2(3H)-ylidene]urea;
N-cyclopentyl-N′-[(2Z)-5-methyl-3-[4-(trifluoromethyl)phenyl]-1,3-thiazol-2(3H)-ylidene]urea;
(3R)—N-[(2Z)-3-(3,5-dichlorophenyl)-5-(hydroxymethyl)-1,3-thiazol-2(3H)-ylidene]-3-fluoropyrrolidine-1-carboxamide;
(3R)—N-[(2Z)-3-(3-chlorophenyl)-5-(hydroxymethyl)-1,3-thiazol-2(3H)-ylidene]-3-fluoropyrrolidine-1-carboxamide;
(3R)—N-[(2Z)-3-(3,4-dimethylphenyl)-5-(hydroxymethyl)-1,3-thiazol-2(3H)-ylidene]-3-fluoropyrrolidine-1-carboxamide;
(3R)—N-[(2Z)-3-(3,5-dimethylphenyl)-5-(hydroxymethyl)-1,3-thiazol-2(3H)-ylidene]-3-fluoropyrrolidine-1-carboxamide;
(3R)—N-[(2Z)-3-(4-chloro-3-methylphenyl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-[4-(trifluoromethoxy)phenyl]-1,3-thiazol-2(3H)-ylidene]pyrrolidine-1-carboxamide;
(3R)—N-[(2Z)-3-(3,4-dichlorophenyl)-5-(hydroxymethyl)-1,3-thiazol-2(3H)-ylidene]-3-fluoropyrrolidine-1-carboxamide;
(3R)-3-fluoro-N-[(2Z)-3-[3-fluoro-4-(trifluoromethyl)phenyl]-5-(hydroxymethyl)-1,3-thiazol-2(3H)-ylidene]pyrrolidine-1-carboxamide;
N-[(2Z)-3-(4-chlorophenyl)-4,5-dimethyl-1,3-thiazol-2(3H)-ylidene]pyrrolidine-1-carboxamide;
N-[(2Z)-4,5-dimethyl-3-[4-(trifluoromethyl)phenyl]-1,3-thiazol-2(3H)-ylidene]pyrrolidine-1-carboxamide;
N-[(2Z)-3-(6-chloropyridin-3-yl)-4,5-dimethyl-1,3-thiazol-2(3H)-ylidene]pyrrolidine-1-carboxamide;
N-[(2Z)-3-(6-chloropyridin-3-yl)-5-methyl-1,3-thiazol-2(3H)-ylidene]pyrrolidine-1-carboxamide;
4-acetyl-N-[(2Z)-5-methyl-3-[4-(trifluoromethyl)phenyl]-1,3-thiazol-2(3H)-ylidene]piperazine-1-carboxamide;
{(2Z)-2-(acetylimino)-3-[4-(trifluoromethyl)phenyl]-2,3-dihydro-1,3-thiazol-5-yl}methyl acetate;
N-[(2Z)-3-(3-methyl-1-benzothien-5-yl)-1,3-thiazol-2(3H)-ylidene]pyrrolidine-1-carboxamide;
N-methyl-N′-[(2Z)-5-methyl-3-[4-(trifluoromethyl)phenyl]-1,3-thiazol-2(3H)-ylidene]-N-pentylurea;
N-[(2′Z)-5-chloro-5′-methyl-2,3′-bi-1,3-thiazol-2′-ylidene]pyrrolidine-1-carboxamide;
N-[(2Z)-3-(4-fluorophenyl)-1,3-thiazol-2(3H)-ylidene]pyrrolidine-1-carboxamide;
N-[(2Z)-3-(5-methylthien-2-yl)-1,3-thiazol-2(3H)-ylidene]pyrrolidine-1-carboxamide;
(3R)—N-[(2Z)-3-(2-chloro-1-benzothien-5-yl)-5-methyl-1,3-thiazol-2(3H)-ylidene]-3-fluoropyrrolidine-1-carboxamide;
(3R)-3-fluoro-N-[(2Z)-5-methyl-3-[1,2,4]triazolo[1,5-a]pyridin-6-yl-1,3-thiazol-2(3H)-ylidene]pyrrolidine-1-carboxamide;
N-[(2Z)-3-[2,4-bis(trifluoromethyl)phenyl]-5-methyl-1,3-thiazol-2(3H)-ylidene]-2,2-dimethylpropanamide;
N-[(2Z)-3-[4-(difluoromethoxy)phenyl]-5-methyl-1,3-thiazol-2(3H)-ylidene]-2,2-dimethylpropanamide;
N-[(2Z)-3-(5-pyridin-2-ylthien-2-yl)-1,3-thiazol-2(3H)-ylidene]pyrrolidine-1-carboxamide;
N-[(2Z)-3-(1-benzofuran-2-yl)-1,3-thiazol-2(3H)-ylidene]pyrrolidine-1-carboxamide;
N-[(2Z)-3-(5-methyl-1-benzothien-2-yl)-1,3-thiazol-2(3H)-ylidene]pyrrolidine-1-carboxamide;
N-[(2Z)-3-thien-2-yl-1,3-thiazol-2(3H)-ylidene]pyrrolidine-1-carboxamide;
N-[(2Z)-3-(2,2′-bithien-5-yl)-1,3-thiazol-2(3H)-ylidene]pyrrolidine-1-carboxamide;
N-[(2Z)-3-(5-cyanothien-2-yl)-1,3-thiazol-2(3H)-ylidene]pyrrolidine-1-carboxamide;
N-[(2Z)-3-(4-chlorophenyl)-1,3-thiazolidin-2-ylidene]-1H-imidazole-1-carboxamide;
(3R)—N-[(2Z)-3-[5-(aminocarbonyl)thien-2-yl]-5-methyl-1,3-thiazol-2(3H)-ylidene]-3-fluoropyrrolidine-1-carboxamide;
N′-[(2Z)-3-(5-chlorothien-2-yl)-5-methyl-1,3-thiazol-2(3H)-ylidene]-N-(3-fluorobenzyl)-N-methylurea;
(3R)—N-[(2Z)-3-(4-cyanophenyl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-(5,6,8-trifluoro-2-naphthyl)-1,3-thiazol-2(3H)-ylidene]pyrrolidine-1-carboxamide;
N-[(2Z)-3-(4-methoxyphenyl)-5-methylene-1,3-thiazolidin-2-ylidene]acetamide;
N-[(2Z)-3-(4-methoxyphenyl)-5-methyl-1,3-thiazol-2(3H)-ylidene]acetamide;
N-{(2Z)-3-[4-(difluoromethoxy)phenyl]-5-methylene-1,3-thiazolidin-2-ylidene}acetamide;
N-[(2Z)-3-[4-(difluoromethoxy)phenyl]-5-methyl-1,3-thiazol-2(3H)-ylidene]acetamide;
N′-[(2Z)-3-[4-(difluoromethoxy)phenyl]-5-methyl-1,3-thiazol-2(3H)-ylidene]-N,N-diisopropylurea;
N′-[(2Z)-3-[4-(difluoromethoxy)phenyl]-5-methyl-1,3-thiazol-2(3H)-ylidene]-N-methyl-N-phenylurea;
N′-[(2Z)-3-[4-(difluoromethoxy)phenyl]-5-methyl-1,3-thiazol-2(3H)-ylidene]-N,N-dimethylurea;
N-{(2Z)-5-methylene-3-[4-(trifluoromethoxy)phenyl]-1,3-thiazolidin-2-ylidene}acetamide;
N-[(2Z)-5-methyl-3-[4-(trifluoromethoxy)phenyl]-1,3-thiazol-2(3H)-ylidene]acetamide;
N-[(2Z)-5-methyl-3-[4-(trifluoromethoxy)phenyl]-1,3-thiazol-2(3H)-ylidene]piperidine-1-carboxamide;
N,N-diisopropyl-N′-[(2Z)-5-methyl-3-[4-(trifluoromethoxy)phenyl]-1,3-thiazol-2(3H)-ylidene]urea;
N-methyl-N′-[(2Z)-5-methyl-3-[4-(trifluoromethoxy)phenyl]-1,3-thiazol-2(3H)-ylidene]-N-phenylurea;
N-[(2Z)-3-(4-chlorophenyl)-5-methylene-1,3-thiazolidin-2-ylidene]acetamide;
N-[(2Z)-3-(4-chlorophenyl)-5-methyl-1,3-thiazol-2(3H)-ylidene]acetamide;
N′-[(2Z)-3-(4-chlorophenyl)-5-methyl-1,3-thiazol-2(3H)-ylidene]-N,N-diisopropylurea;
N-[(2Z)-3-(6,7-dicyano-2-naphthyl)-5-methyl-1,3-thiazol-2(3H)-ylidene]-2,2-dimethylpropanamide;
N-[(2Z)-3-(4-benzylphenyl)-5-methyl-1,3-thiazol-2(3H)-ylidene]-2,2-dimethylpropanamide;
N′-[(2Z)-3-(4-fluorophenyl)-5-methyl-1,3-thiazol-2(3H)-ylidene]-N,N-diisopropylurea;
N-[(2Z)-3-[4-(cyanomethyl)phenyl]-5-methyl-1,3-thiazol-2(3H)-ylidene]pyrrolidine-1-carboxamide;
N-[(2Z)-3-[4-(cyanomethyl)phenyl]-5-methyl-1,3-thiazol-2(3H)-ylidene]piperidine-1-carboxamide;
N′-[(2Z)-3-[4-(cyanomethyl)phenyl]-5-methyl-1,3-thiazol-2(3H)-ylidene]-N,N-diethylurea;
N′-[(2Z)-3-[4-(cyanomethyl)phenyl]-5-methyl-1,3-thiazol-2(3H)-ylidene]-N,N-dimethylurea;
N-[(2Z)-5-methyl-3-[4-(trifluoromethyl)phenyl]-1,3-thiazol-2(3H)-ylidene]piperidine-1-carboxamide;
N-[(2Z)-3-[4-(cyanomethyl)phenyl]-5-methyl-1,3-thiazol-2(3H)-ylidene]-2,2-dimethylpropanamide;
N-[(2Z)-3-(2,6-dimethylpyrimidin-4-yl)-5-methyl-1,3-thiazol-2(3H)-ylidene]-2,2-dimethylpropanamide;
N-[(2Z)-5-methyl-3-[4-(trifluoromethyl)phenyl]-1,3-thiazol-2(3H)-ylidene]-1H-imidazole-1-carboxamide;
tert-butyl (2R,3S)-2-methyl-1-({[(2Z)-5-methyl-3-[4-(trifluoromethyl)phenyl]-1,3-thiazol-2(3H)-ylidene]amino}carbonyl)pyrrolidin-3-ylcarbamate;
2-isopropyl-N-[(2Z)-5-methyl-3-[4-(trifluoromethyl)phenyl]-1,3-thiazol-2(3H)-ylidene]pyrrolidine-1-carboxamide;
2-isobutyl-N-[(2Z)-5-methyl-3-[4-(trifluoromethyl)phenyl]-1,3-thiazol-2(3H)-ylidene]pyrrolidine-1-carboxamide;
(3R)-3-(dimethylamino)-N-[(2Z)-5-methyl-3-[4-(trifluoromethyl)phenyl]-1,3-thiazol-2(3H)-ylidene]pyrrolidine-1-carboxamide;
tert-butyl (3S)-1-({[(2Z)-5-methyl-3-[4-(trifluoromethyl)phenyl]-1,3-thiazol-2(3H)-ylidene]amino}carbonyl)pyrrolidin-3-ylcarbamate;
tert-butyl 5-({[(2Z)-5-methyl-3-[4-(trifluoromethyl)phenyl]-1,3-thiazol-2(3H)-ylidene]amino}carbonyl)hexahydropyrrolo[3,4-c]pyrrole-2(1H)-carboxylate;
tert-butyl (1S,5R)-3-({[(2Z)-5-methyl-3-[4-(trifluoromethyl)phenyl]-1,3-thiazol-2(3H)-ylidene]amino}carbonyl)-3,6-diazabicyclo[3.2.0]heptane-6-carboxylate;
tert-butyl (3R)-3-methyl-1-({[(2Z)-5-methyl-3-[4-(trifluoromethyl)phenyl]-1,3-thiazol-2(3H)-ylidene]amino}carbonyl)pyrrolidin-3-ylcarbamate;
tert-butyl 6-({[(2Z)-5-methyl-3-[4-(trifluoromethyl)phenyl]-1,3-thiazol-2(3H)-ylidene]amino}carbonyl)octahydro-1H-pyrrolo[3,4-b]pyridine-1-carboxylate;
tert-butyl (1S,4S)-5-({[(2Z)-5-methyl-3-[4-(trifluoromethyl)phenyl]-1,3-thiazol-2(3H)-ylidene]amino}carbonyl)-2,5-diazabicyclo[2.2.1]heptane-2-carboxylate;
(3S)-3-amino-N-[(2Z)-5-methyl-3-[4-(trifluoromethyl)phenyl]-1,3-thiazol-2(3H)-ylidene]pyrrolidine-1-carboxamide;
(2R,3S)-3-amino-2-methyl-N-[(2Z)-5-methyl-3-[4-(trifluoromethyl)phenyl]-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]hexahydropyrrolo[3,4-c]pyrrole-2(1H)-carboxamide;
(3R)-3-amino-3-methyl-N-[(2Z)-5-methyl-3-[4-(trifluoromethyl)phenyl]-1,3-thiazol-2(3H)-ylidene]pyrrolidine-1-carboxamide;
(1R,5R)—N-[(2Z)-5-methyl-3-[4-(trifluoromethyl)phenyl]-1,3-thiazol-2(3H)-ylidene]-3,6-diazabicyclo[3.2.0]heptane-3-carboxamide;
(4aS,7aS)—N-[(2Z)-5-methyl-3-[4-(trifluoromethyl)phenyl]-1,3-thiazol-2(3H)-ylidene]octahydro-6H-pyrrolo[3,4-b]pyridine-6-carboxamide;
(1S,4S)—N-[(2Z)-5-methyl-3-[4-(trifluoromethyl)phenyl]-1,3-thiazol-2(3H)-ylidene]-2,5-diazabicyclo[2.2.1]heptane-2-carboxamide;
N′-[(2Z)-3-(4-chlorophenyl)-4,5-dimethyl-1,3-thiazol-2(3H)-ylidene]-N,N-dimethylurea;
N′-[(2Z)-4,5-dimethyl-3-[4-(trifluoromethyl)phenyl]-1,3-thiazol-2(3H)-ylidene]-N,N-dimethylurea;
N′-[(2Z)-3-(3-ethoxyphenyl)-4,5-dimethyl-1,3-thiazol-2(3H)-ylidene]-N,N-dimethylurea;
N-[(2Z)-3-(3-ethoxyphenyl)-4,5-dimethyl-1,3-thiazol-2(3H)-ylidene]pyrrolidine-1-carboxamide;
N′-[(2Z)-3-(4-chlorophenyl)-4-phenyl-1,3-thiazol-2(3H)-ylidene]-N,N-dimethylurea;
N-isopropyl-N-methyl-N′-[(2Z)-5-methyl-3-[4-(trifluoromethyl)phenyl]-1,3-thiazol-2(3H)-ylidene]urea;
N-butyl-N-methyl-N′-[(2Z)-5-methyl-3-[4-(trifluoromethyl)phenyl]-1,3-thiazol-2(3H)-ylidene]urea;
N-isobutyl-N-methyl-N′-[(2Z)-5-methyl-3-[4-(trifluoromethyl)phenyl]-1,3-thiazol-2(3H)-ylidene]urea;
N-cyclohexyl-N-methyl-N′-[(2Z)-5-methyl-3-[4-(trifluoromethyl)phenyl]-1,3-thiazol-2(3H)-ylidene]urea;
N-ethyl-N-isopropyl-N′-[(2Z)-5-methyl-3-[4-(trifluoromethyl)phenyl]-1,3-thiazol-2(3H)-ylidene]urea;
N-cyclohexyl-N-ethyl-N′-[(2Z)-5-methyl-3-[4-(trifluoromethyl)phenyl]-1,3-thiazol-2(3H)-ylidene]urea;
N′-[(2Z)-5-methyl-3-[4-(trifluoromethyl)phenyl]-1,3-thiazol-2(3H)-ylidene]-N,N-dipropylurea;
N-isopropyl-N′-[(2Z)-5-methyl-3-[4-(trifluoromethyl)phenyl]-1,3-thiazol-2(3H)-ylidene]-N-propylurea;
N-butyl-N′-[(2Z)-5-methyl-3-[4-(trifluoromethyl)phenyl]-1,3-thiazol-2(3H)-ylidene]-N-propylurea;
N-(sec-butyl)-N′-[(2Z)-5-methyl-3-[4-(trifluoromethyl)phenyl]-1,3-thiazol-2(3H)-ylidene]-N-propylurea;
N-(cyclopropylmethyl)-N′-[(2Z)-5-methyl-3-[4-(trifluoromethyl)phenyl]-1,3-thiazol-2(3H)-ylidene]-N-propylurea;
N-(cyanomethyl)-N-methyl-N′-[(2Z)-5-methyl-3-[4-(trifluoromethyl)phenyl]-1,3-thiazol-2(3H)-ylidene]urea;
N-(2-cyanoethyl)-N-cyclopropyl-N′-[(2Z)-5-methyl-3-[4-(trifluoromethyl)phenyl]-1,3-thiazol-2(3H)-ylidene]urea;
N,N-diisobutyl-N′-[(2Z)-5-methyl-3-[4-(trifluoromethyl)phenyl]-1,3-thiazol-2(3H)-ylidene]urea;
3-methyl-N-[(2Z)-5-methyl-3-[4-(trifluoromethyl)phenyl]-1,3-thiazol-2(3H)-ylidene]piperidine-1-carboxamide;
4-methyl-N-[(2Z)-5-methyl-3-[4-(trifluoromethyl)phenyl]-1,3-thiazol-2(3H)-ylidene]piperidine-1-carboxamide;
2-ethyl-N-[(2Z)-5-methyl-3-[4-(trifluoromethyl)phenyl]-1,3-thiazol-2(3H)-ylidene]piperidine-1-carboxamide;
N-[(2Z)-5-methyl-3-[4-(trifluoromethyl)phenyl]-1,3-thiazol-2(3H)-ylidene]-4-propylpiperidine-1-carboxamide;
N-[(2Z)-5-methyl-3-[4-(trifluoromethyl)phenyl]-1,3-thiazol-2(3H)-ylidene]azepane-1-carboxamide;
N,N-bis(2-ethoxyethyl)-N′-[(2Z)-5-methyl-3-[4-(trifluoromethyl)phenyl]-1,3-thiazol-2(3H)-ylidene]urea;
N-ethyl-N-(2-methoxyethyl)-N′-[(2Z)-5-methyl-3-[4-(trifluoromethyl)phenyl]-1,3-thiazol-2(3H)-ylidene]urea;
N-(2-methoxyethyl)-N′-[(2Z)-5-methyl-3-[4-(trifluoromethyl)phenyl]-1,3-thiazol-2(3H)-ylidene]-N-propylurea;
N-isopropyl-N-(2-methoxyethyl)-N′-[(2Z)-5-methyl-3-[4-(trifluoromethyl)phenyl]-1,3-thiazol-2(3H)-ylidene]urea;
N,N-bis(2-methoxyethyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]morpholine-4-carboxamide;
N-[(2Z)-5-methyl-3-[4-(trifluoromethyl)phenyl]-1,3-thiazol-2(3H)-ylidene]thiomorpholine-4-carboxamide;
N-(2-cyanoethyl)-N-methyl-N′-[(2Z)-5-methyl-3-[4-(trifluoromethyl)phenyl]-1,3-thiazol-2(3H)-ylidene]urea;
N-benzyl-N′-[(2Z)-5-methyl-3-[4-(trifluoromethyl)phenyl]-1,3-thiazol-2(3H)-ylidene]urea;
N-(3-chlorobenzyl)-N′-[(2Z)-5-methyl-3-[4-(trifluoromethyl)phenyl]-1,3-thiazol-2(3H)-ylidene]urea;
N-(3,5-dichlorobenzyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-(2-phenylethyl)urea;
N-[(2Z)-5-methyl-3-[4-(trifluoromethyl)phenyl]-1,3-thiazol-2(3H)-ylidene]-N′-(2-pyridin-2-ylethyl)urea;
N-[(2Z)-5-methyl-3-[4-(trifluoromethyl)phenyl]-1,3-thiazol-2(3H)-ylidene]-N′-(2-pyridin-3-ylethyl)urea;
N-[(2Z)-5-methyl-3-[4-(trifluoromethyl)phenyl]-1,3-thiazol-2(3H)-ylidene]-N′-(2-pyridin-4-ylethyl)urea;
N-[(2Z)-5-methyl-3-[4-(trifluoromethyl)phenyl]-1,3-thiazol-2(3H)-ylidene]-N′-(pyridin-3-ylmethyl)urea;
N-[2-(dimethylamino)ethyl]-N′-[(2Z)-5-methyl-3-[4-(trifluoromethyl)phenyl]-1,3-thiazol-2(3H)-ylidene]urea;
N-[3-(dimethylamino)propyl]-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-(2-pyrrolidin-1-ylethyl)urea;
N-[(2Z)-5-methyl-3-[4-(trifluoromethyl)phenyl]-1,3-thiazol-2(3H)-ylidene]-N′-(3-pyrrolidin-1-ylpropyl)urea;
N-[2-(4-methylpiperazin-1-yl)ethyl]-N′-[(2Z)-5-methyl-3-[4-(trifluoromethyl)phenyl]-1,3-thiazol-2(3H)-ylidene]urea;
N-[2-(dimethylamino)ethyl]-N-methyl-N′-[(2Z)-5-methyl-3-[4-(trifluoromethyl)phenyl]-1,3-thiazol-2(3H)-ylidene]urea;
N-[3-(dimethylamino)propyl]-N-methyl-N′-[(2Z)-5-methyl-3-[4-(trifluoromethyl)phenyl]-1,3-thiazol-2(3H)-ylidene]urea;
N-[2-(dimethylamino)ethyl]-N-ethyl-N′-[(2Z)-5-methyl-3-[4-(trifluoromethyl)phenyl]-1,3-thiazol-2(3H)-ylidene]urea;
N-[2-(diethylamino)ethyl]-N-methyl-N′-[(2Z)-5-methyl-3-[4-(trifluoromethyl)phenyl]-1,3-thiazol-2(3H)-ylidene]urea;
N,N-dibenzyl-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-(2-phenylethyl)urea;
N-methyl-N′-[(2Z)-5-methyl-3-[4-(trifluoromethyl)phenyl]-1,3-thiazol-2(3H)-ylidene]-N-(2-pyridin-2-ylethyl)urea;
4-hydroxy-N-[(2Z)-5-methyl-3-[4-(trifluoromethyl)phenyl]-1,3-thiazol-2(3H)-ylidene]piperidine-1-carboxamide;
2,6-dimethyl-N-[(2Z)-5-methyl-3-[4-(trifluoromethyl)phenyl]-1,3-thiazol-2(3H)-ylidene]morpholine-4-carboxamide;
4-methyl-N-[(2Z)-5-methyl-3-[4-(trifluoromethyl)phenyl]-1,3-thiazol-2(3H)-ylidene]piperazine-1-carboxamide;
4-ethyl-N-[(2Z)-5-methyl-3-[4-(trifluoromethyl)phenyl]-1,3-thiazol-2(3H)-ylidene]piperazine-1-carboxamide;
N-[(2Z)-5-methyl-3-[4-(trifluoromethyl)phenyl]-1,3-thiazol-2(3H)-ylidene]-4-phenylpiperazine-1-carboxamide;
N-[(2Z)-5-methyl-3-[4-(trifluoromethyl)phenyl]-1,3-thiazol-2(3H)-ylidene]-4-pyridin-2-ylpiperazine-1-carboxamide;
4-methyl-N-[(2Z)-5-methyl-3-[4-(trifluoromethyl)phenyl]-1,3-thiazol-2(3H)-ylidene]-1,4-diazepane-1-carboxamide;
N-[(2Z)-5-methyl-3-[4-(trifluoromethyl)phenyl]-1,3-thiazol-2(3H)-ylidene]-4-pyrimidin-2-ylpiperazine-1-carboxamide;
N-isobutyl-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-pentylurea;
N-(3-methylbutyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-neopentylurea;
N-(2-methoxyethyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-[(2S)-tetrahydrofuran-2-ylmethyl]urea;
N-[(2Z)-5-methyl-3-[4-(trifluoromethyl)phenyl]-1,3-thiazol-2(3H)-ylidene]-N′-[(2R)-tetrahydrofuran-2-ylmethyl]urea;
N-[(2Z)-5-methyl-3-[4-(trifluoromethyl)phenyl]-1,3-thiazol-2(3H)-ylidene]-N′-(tetrahydrofuran-3-ylmethyl)urea;
N-[(2Z)-5-methyl-3-[4-(trifluoromethyl)phenyl]-1,3-thiazol-2(3H)-ylidene]-N′-prop-2-ynylurea;
N-(cyclopropylmethyl)-N′-[(2Z)-5-methyl-3-[4-(trifluoromethyl)phenyl]-1,3-thiazol-2(3H)-ylidene]urea;
N-(cyclopentylmethyl)-N′-[(2Z)-5-methyl-3-[4-(trifluoromethyl)phenyl]-1,3-thiazol-2(3H)-ylidene]urea;
N-cyclohexyl-N′-[(2Z)-5-methyl-3-[4-(trifluoromethyl)phenyl]-1,3-thiazol-2(3H)-ylidene]urea;
N-cycloheptyl-N′-[(2Z)-5-methyl-3-[4-(trifluoromethyl)phenyl]-1,3-thiazol-2(3H)-ylidene]urea;
N-1-adamantyl-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-phenylurea;
N-(2-methylphenyl)-N′-[(2Z)-5-methyl-3-[4-(trifluoromethyl)phenyl]-1,3-thiazol-2(3H)-ylidene]urea;
N-(3-methylphenyl)-N′-[(2Z)-5-methyl-3-[4-(trifluoromethyl)phenyl]-1,3-thiazol-2(3H)-ylidene]urea;
N-(4-methylphenyl)-N′-[(2Z)-5-methyl-3-[4-(trifluoromethyl)phenyl]-1,3-thiazol-2(3H)-ylidene]urea;
N-(3-methoxyphenyl)-N′-[(2Z)-5-methyl-3-[4-(trifluoromethyl)phenyl]-1,3-thiazol-2(3H)-ylidene]urea;
N-(3-fluorophenyl)-N′-[(2Z)-5-methyl-3-[4-(trifluoromethyl)phenyl]-1,3-thiazol-2(3H)-ylidene]urea;
N-(3-chlorophenyl)-N′-[(2Z)-5-methyl-3-[4-(trifluoromethyl)phenyl]-1,3-thiazol-2(3H)-ylidene]urea;
N-(3-bromophenyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-[3-(trifluoromethyl)phenyl]urea;
N-(3,5-dichlorophenyl)-N′-[(2Z)-5-methyl-3-[4-(trifluoromethyl)phenyl]-1,3-thiazol-2(3H)-ylidene]urea;
N-methyl-N-(4-methylphenyl)-N′-[(2Z)-5-methyl-3-[4-(trifluoromethyl)phenyl]-1,3-thiazol-2(3H)-ylidene]urea;
N-{(2Z)-5-methyl-3-[4-(trifluoromethyl)phenyl]-1,3-thiazolidin-2-ylidene}acetamide;
N-[(2Z)-4,5-dimethyl-3-(6-morpholin-4-ylpyridin-3-yl)-1,3-thiazol-2(3H)-ylidene]pyrrolidine-1-carboxamide;
N-{(2Z)-5-methylene-3-[4-(trifluoromethyl)phenyl]-1,3-thiazolidin-2-ylidene}pyrrolidine-1-carboxamide;
N-{(2Z)-4-hydroxy-5-methylene-3-[4-(trifluoromethyl)phenyl]-1,3-thiazolidin-2-ylidene}pyrrolidine-1-carboxamide;
N-{(2Z)-5-methylene-4-(1H-pyrazol-1-yl)-3-[4-(trifluoromethyl)phenyl]-1,3-thiazolidin-2-ylidene}acetamide;
N-[(2Z)-5-(1H-pyrazol-1-ylmethyl)-3-[4-(trifluoromethyl)phenyl]-1,3-thiazol-2(3H)-ylidene]acetamide;
N-{(2Z,5Z)-5-(iodomethylene)-3-[4-(trifluoromethyl)phenyl]-1,3-thiazolidin-2-ylidene}acetamide;
N-[(2Z)-5-(cyanomethyl)-3-[4-(trifluoromethyl)phenyl]-1,3-thiazol-2(3H)-ylidene]acetamide;
N-[(2Z)-5-[(ethylamino)methyl]-3-[4-(trifluoromethyl)phenyl]-1,3-thiazol-2(3H)-ylidene]acetamide;
N-[(2Z)-5-[(propylamino)methyl]-3-[4-(trifluoromethyl)phenyl]-1,3-thiazol-2(3H)-ylidene]acetamide;
N-[(2Z)-5-[(butylamino)methyl]-3-[4-(trifluoromethyl)phenyl]-1,3-thiazol-2(3H)-ylidene]acetamide;
N-[(2Z)-5-[(tert-butylamino)methyl]-3-[4-(trifluoromethyl)phenyl]-1,3-thiazol-2(3H)-ylidene]acetamide;
N-[(2Z)-5-{[(cyanomethyl)amino]methyl}-3-[4-(trifluoromethyl)phenyl]-1,3-thiazol-2(3H)-ylidene]acetamide;
N-[(2Z)-5-[(dimethylamino)methyl]-3-[4-(trifluoromethyl)phenyl]-1,3-thiazol-2(3H)-ylidene]acetamide;
N-[(2Z)-5-{[butyl(methyl)amino]methyl}-3-[4-(trifluoromethyl)phenyl]-1,3-thiazol-2(3H)-ylidene]acetamide;
N-[(2Z)-5-{[(1,3-dioxolan-2-ylmethyl)(methyl)amino]methyl}-3-[4-(trifluoromethyl)phenyl]-1,3-thiazol-2(3H)-ylidene]acetamide;
N-[(2Z)-5-[(diethylamino)methyl]-3-[4-(trifluoromethyl)phenyl]-1,3-thiazol-2(3H)-ylidene]acetamide;
N-[(2Z)-5-(azetidin-1-ylmethyl)-3-[4-(trifluoromethyl)phenyl]-1,3-thiazol-2(3H)-ylidene]acetamide;
N-[(2Z)-5-{[bis(2-methoxyethyl)amino]methyl}-3-[4-(trifluoromethyl)phenyl]-1,3-thiazol-2(3H)-ylidene]acetamide;
N-[(2Z)-5-{[(1-phenylethyl)amino]methyl}-3-[4-(trifluoromethyl)phenyl]-1,3-thiazol-2(3H)-ylidene]acetamide;
N-[(2Z)-5-methyl-3-(2-methylphenyl)-1,3-thiazol-2(3H)-ylidene]pyrrolidine-1-carboxamide;
N-[(2Z)-3-(2-aminophenyl)-5-methyl-1,3-thiazol-2(3H)-ylidene]pyrrolidine-1-carboxamide;
N-[(2Z)-3-(2-hydroxyphenyl)-5-methyl-1,3-thiazol-2(3H)-ylidene]pyrrolidine-1-carboxamide;
N-[(2Z)-3-(4-hydroxyphenyl)-5-methyl-1,3-thiazol-2(3H)-ylidene]pyrrolidine-1-carboxamide;
N-[(2Z)-3-(2-methoxyphenyl)-5-methyl-1,3-thiazol-2(3H)-ylidene]pyrrolidine-1-carboxamide;
N-[(2Z)-3-(2-fluorophenyl)-5-methyl-1,3-thiazol-2(3H)-ylidene]pyrrolidine-1-carboxamide;
N-[(2Z)-3-(2-chlorophenyl)-5-methyl-1,3-thiazol-2(3H)-ylidene]pyrrolidine-1-carboxamide;
N-[(2Z)-3-[3-(dimethylamino)phenyl]-5-methyl-1,3-thiazol-2(3H)-ylidene]pyrrolidine-1-carboxamide;
N-[(2Z)-3-(2,3-dimethylphenyl)-5-methyl-1,3-thiazol-2(3H)-ylidene]pyrrolidine-1-carboxamide;
N-[(2Z)-3-(2,4-dimethylphenyl)-5-methyl-1,3-thiazol-2(3H)-ylidene]pyrrolidine-1-carboxamide;
N-[(2Z)-3-(2,5-dimethylphenyl)-5-methyl-1,3-thiazol-2(3H)-ylidene]pyrrolidine-1-carboxamide;
N-[(2Z)-3-(2,4-dimethoxyphenyl)-5-methyl-1,3-thiazol-2(3H)-ylidene]pyrrolidine-1-carboxamide;
N-[(2Z)-3-(2,5-dimethoxyphenyl)-5-methyl-1,3-thiazol-2(3H)-ylidene]pyrrolidine-1-carboxamide;
N-[(2Z)-3-(3,4-dimethoxyphenyl)-5-methyl-1,3-thiazol-2(3H)-ylidene]pyrrolidine-1-carboxamide;
N-[(2Z)-5-methyl-3-(3,4,5-trimethoxyphenyl)-1,3-thiazol-2(3H)-ylidene]pyrrolidine-1-carboxamide;
N-[(2Z)-3-(2,3-dichlorophenyl)-5-methyl-1,3-thiazol-2(3H)-ylidene]pyrrolidine-1-carboxamide;
N-[(2Z)-3-(2,4-dichlorophenyl)-5-methyl-1,3-thiazol-2(3H)-ylidene]pyrrolidine-1-carboxamide;
N-[(2Z)-3-(2,5-dichlorophenyl)-5-methyl-1,3-thiazol-2(3H)-ylidene]pyrrolidine-1-carboxamide;
N-[(2Z)-3-(4-aminophenyl)-5-methyl-1,3-thiazol-2(3H)-ylidene]pyrrolidine-1-carboxamide;
N-[(2Z)-5-methyl-3-(3-propionylphenyl)-1,3-thiazol-2(3H)-ylidene]pyrrolidine-1-carboxamide;
N-[(2Z)-3-(4-tert-butylphenyl)-5-methyl-1,3-thiazol-2(3H)-ylidene]pyrrolidine-1-carboxamide;
N-[(2Z)-3-(4-ethylphenyl)-5-methyl-1,3-thiazol-2(3H)-ylidene]pyrrolidine-1-carboxamide;
N-[(2Z)-5-methyl-3-(4-propylphenyl)-1,3-thiazol-2(3H)-ylidene]pyrrolidine-1-carboxamide;
N-[(2Z)-5-methyl-3-pyridin-3-yl-1,3-thiazol-2(3H)-ylidene]pyrrolidine-1-carboxamide;
N-[(2Z)-3-(4-butylphenyl)-5-methyl-1,3-thiazol-2(3H)-ylidene]pyrrolidine-1-carboxamide;
N-[(2Z)-5-methyl-3-[3-(methylthio)phenyl]-1,3-thiazol-2(3H)-ylidene]pyrrolidine-1-carboxamide;
N-[(2Z)-5-methyl-3-[2,3,5,6-tetrafluoro-4-(trifluoromethyl)phenyl]-1,3-thiazol-2(3H)-ylidene]pyrrolidine-1-carboxamide;
N-[(2Z)-3-(3-isopropoxyphenyl)-5-methyl-1,3-thiazol-2(3H)-ylidene]pyrrolidine-1-carboxamide;
N-[(2Z)-3-[2,4-bis(trifluoromethyl)phenyl]-5-methyl-1,3-thiazol-2(3H)-ylidene]pyrrolidine-1-carboxamide;
N-[(2Z)-3-(4-chloro-3-methoxyphenyl)-5-methyl-1,3-thiazol-2(3H)-ylidene]pyrrolidine-1-carboxamide;
N-[(2Z)-5-methyl-3-(1-oxo-2,3-dihydro-1H-inden-5-yl)-1,3-thiazol-2(3H)-ylidene]pyrrolidine-1-carboxamide;
N-[(2Z)-3-(4-methoxy-3-methylphenyl)-5-methyl-1,3-thiazol-2(3H)-ylidene]pyrrolidine-1-carboxamide;
N-[(2Z)-3-(4-cyclohexylphenyl)-5-methyl-1,3-thiazol-2(3H)-ylidene]pyrrolidine-1-carboxamide;
N-[(2Z)-5-methyl-3-(5-phenylthien-2-yl)-1,3-thiazol-2(3H)-ylidene]pyrrolidine-1-carboxamide;
N-[(2Z)-5-methyl-3-(1-naphthyl)-1,3-thiazol-2(3H)-ylidene]pyrrolidine-1-carboxamide;
N-[(2Z)-5-methyl-3-(6-methylpyridazin-3-yl)-1,3-thiazol-2(3H)-ylidene]pyrrolidine-1-carboxamide;
N-[(2Z)-5-methyl-3-(6-methylpyrazin-2-yl)-1,3-thiazol-2(3H)-ylidene]pyrrolidine-1-carboxamide;
N-[(2Z)-5-methyl-3-pyrazin-2-yl-1,3-thiazol-2(3H)-ylidene]pyrrolidine-1-carboxamide;
N-[(2Z)-5-methyl-3-quinoxalin-2-yl-1,3-thiazol-2(3H)-ylidene]pyrrolidine-1-carboxamide;
N-[(2Z)-5-methyl-3-[5-(trifluoromethyl)pyridin-2-yl]-1,3-thiazol-2(3H)-ylidene]pyrrolidine-1-carboxamide;
N-[(2Z)-5-methyl-3-[6-(trifluoromethyl)pyridin-2-yl]-1,3-thiazol-2(3H)-ylidene]pyrrolidine-1-carboxamide;
N-[(2Z)-5-methyl-3-[4-(trifluoromethyl)pyridin-2-yl]-1,3-thiazol-2(3H)-ylidene]pyrrolidine-1-carboxamide;
N-[(2Z)-3-(4′-fluoro-1,1′-biphenyl-4-yl)-5-methyl-1,3-thiazol-2(3H)-ylidene]pyrrolidine-1-carboxamide;
N-[(2Z)-3-(4-acetylphenyl)-1,3-thiazol-2(3H)-ylidene]pyrrolidine-1-carboxamide;
N-[(2Z)-3-(4-propionylphenyl)-1,3-thiazol-2(3H)-ylidene]pyrrolidine-1-carboxamide;
N-[(2Z)-3-(4-tert-butylphenyl)-1,3-thiazol-2(3H)-ylidene]pyrrolidine-1-carboxamide;
N-[(2Z)-3-(4-ethylphenyl)-1,3-thiazol-2(3H)-ylidene]pyrrolidine-1-carboxamide;
N-[(2Z)-3-(2,4-dichlorophenyl)-1,3-thiazol-2(3H)-ylidene]pyrrolidine-1-carboxamide;
N-[(2Z)-3-(2-methoxyphenyl)-1,3-thiazol-2(3H)-ylidene]pyrrolidine-1-carboxamide;
N-[(2Z)-3-(2-methylphenyl)-1,3-thiazol-2(3H)-ylidene]pyrrolidine-1-carboxamide;
N-[(2Z)-3-(3,5-dichlorophenyl)-1,3-thiazol-2(3H)-ylidene]pyrrolidine-1-carboxamide;
N-[(2Z)-3-(3-methoxyphenyl)-1,3-thiazol-2(3H)-ylidene]pyrrolidine-1-carboxamide;
N-[(2Z)-3-(3-methylphenyl)-1,3-thiazol-2(3H)-ylidene]pyrrolidine-1-carboxamide;
N-[(2Z)-3-(4-methoxyphenyl)-1,3-thiazol-2(3H)-ylidene]pyrrolidine-1-carboxamide;
N-[(2Z)-3-(4-propylphenyl)-1,3-thiazol-2(3H)-ylidene]pyrrolidine-1-carboxamide;
N-[(2Z)-3-(2,4-dimethylphenyl)-1,3-thiazol-2(3H)-ylidene]pyrrolidine-1-carboxamide;
N-[(2Z)-3-pyridin-3-yl-1,3-thiazol-2(3H)-ylidene]pyrrolidine-1-carboxamide;
N-[(2Z)-3-(4-butylphenyl)-1,3-thiazol-2(3H)-ylidene]pyrrolidine-1-carboxamide;
N-[(2Z)-3-[3-(methylthio)phenyl]-1,3-thiazol-2(3H)-ylidene]pyrrolidine-1-carboxamide;
N-[(2Z)-3-(2,3-dichlorophenyl)-1,3-thiazol-2(3H)-ylidene]pyrrolidine-1-carboxamide;
N-[(2Z)-3-(2-chlorophenyl)-1,3-thiazol-2(3H)-ylidene]pyrrolidine-1-carboxamide;
N-[(2Z)-3-[2,3,5,6-tetrafluoro-4-(trifluoromethyl)phenyl]-1,3-thiazol-2(3H)-ylidene]pyrrolidine-1-carboxamide;
N-[(2Z)-3-(3,5-dimethylphenyl)-1,3-thiazol-2(3H)-ylidene]pyrrolidine-1-carboxamide;
N-[(2Z)-3-(3-isopropoxyphenyl)-1,3-thiazol-2(3H)-ylidene]pyrrolidine-1-carboxamide;
N-[(2Z)-3-[2,4-bis(trifluoromethyl)phenyl]-1,3-thiazol-2(3H)-ylidene]pyrrolidine-1-carboxamide;
N-[(2Z)-3-(3-cyanophenyl)-1,3-thiazol-2(3H)-ylidene]pyrrolidine-1-carboxamide;
N-[(2Z)-3-(1-oxo-2,3-dihydro-1H-inden-5-yl)-1,3-thiazol-2(3H)-ylidene]pyrrolidine-1-carboxamide;
N-[(2Z)-3-(4-methoxy-3-methylphenyl)-1,3-thiazol-2(3H)-ylidene]pyrrolidine-1-carboxamide;
N-[(2Z)-3-[4-(dimethylamino)phenyl]-5-methyl-1,3-thiazol-2(3H)-ylidene]-2,2-dimethylpropanamide;
2,2-dimethyl-N-[(2Z)-5-methyl-3-[4-(methylthio)phenyl]-1,3-thiazol-2(3H)-ylidene]propanamide;
N-[(2Z)-3-(4-acetylphenyl)-5-methyl-1,3-thiazol-2(3H)-ylidene]-2,2-dimethylpropanamide;
N-[(2Z)-3-(4-tert-butylphenyl)-5-methyl-1,3-thiazol-2(3H)-ylidene]-2,2-dimethylpropanamide;
N-[(2Z)-3-(4-ethylphenyl)-5-methyl-1,3-thiazol-2(3H)-ylidene]-2,2-dimethylpropanamide;
2,2-dimethyl-N-[(2Z)-5-methyl-3-(2-methylphenyl)-1,3-thiazol-2(3H)-ylidene]propanamide;
N-[(2Z)-3-[3-(dimethylamino)phenyl]-5-methyl-1,3-thiazol-2(3H)-ylidene]-2,2-dimethylpropanamide;
2,2-dimethyl-N-[(2Z)-5-methyl-3-phenyl-1,3-thiazol-2(3H)-ylidene]propanamide;
N-[(2Z)-3-(2-methoxyphenyl)-5-methyl-1,3-thiazol-2(3H)-ylidene]-2,2-dimethylpropanamide;
N-[(2Z)-3-(3-methoxyphenyl)-5-methyl-1,3-thiazol-2(3H)-ylidene]-2,2-dimethylpropanamide;
2,2-dimethyl-N-[(2Z)-5-methyl-3-(3-methylphenyl)-1,3-thiazol-2(3H)-ylidene]propanamide;
N-[(2Z)-3-(4-methoxyphenyl)-5-methyl-1,3-thiazol-2(3H)-ylidene]-2,2-dimethylpropanamide;
2,2-dimethyl-N-[(2Z)-5-methyl-3-(4-methylphenyl)-1,3-thiazol-2(3H)-ylidene]propanamide;
2,2-dimethyl-N-[(2Z)-5-methyl-3-(2-naphthyl)-1,3-thiazol-2(3H)-ylidene]propanamide;
N-[(2Z)-3-(6-methoxy-2-naphthyl)-5-methyl-1,3-thiazol-2(3H)-ylidene]-2,2-dimethylpropanamide;
N-[(2Z)-3-(2,4-dimethylphenyl)-5-methyl-1,3-thiazol-2(3H)-ylidene]-2,2-dimethylpropanamide;
N-[(2Z)-3-(4-chloro-3-methylphenyl)-5-methyl-1,3-thiazol-2(3H)-ylidene]-2,2-dimethylpropanamide;
N-[(2Z)-3-(4-butylphenyl)-5-methyl-1,3-thiazol-2(3H)-ylidene]-2,2-dimethylpropanamide;
N-[(2Z)-3-(3,4-dimethylphenyl)-5-methyl-1,3-thiazol-2(3H)-ylidene]-2,2-dimethylpropanamide;
2,2-dimethyl-N-[(2Z)-5-methyl-3-[3-(methylthio)phenyl]-1,3-thiazol-2(3H)-ylidene]propanamide;
N-[(2Z)-3-(3-chloro-4-methylphenyl)-5-methyl-1,3-thiazol-2(3H)-ylidene]-2,2-dimethylpropanamide;
N-[(2Z)-3-(3,5-dimethylphenyl)-5-methyl-1,3-thiazol-2(3H)-ylidene]-2,2-dimethylpropanamide;
N-[(2Z)-3-(3-isopropoxyphenyl)-5-methyl-1,3-thiazol-2(3H)-ylidene]-2,2-dimethylpropanamide;
N-[(2Z)-3-(4-chloro-3-methoxyphenyl)-5-methyl-1,3-thiazol-2(3H)-ylidene]-2,2-dimethylpropanamide;
N-[(2Z)-3-(4-methoxy-3-methylphenyl)-5-methyl-1,3-thiazol-2(3H)-ylidene]-2,2-dimethylpropanamide;
N-[(2Z)-3-(2-chlorophenyl)-5-methyl-1,3-thiazol-2(3H)-ylidene]-2,2-dimethylpropanamide;
2,2-dimethyl-N-[(2Z)-5-methyl-3-[2,3,5,6-tetrafluoro-4-(trifluoromethyl)phenyl]-1,3-thiazol-2(3H)-ylidene]propanamide;
2,2-dimethyl-N-[(2Z)-5-methyl-3-pyridin-3-yl-1,3-thiazol-2(3H)-ylidene]propanamide;
N-[(2Z)-3-(4-cyanophenyl)-5-methyl-1,3-thiazol-2(3H)-ylidene]-2,2-dimethylpropanamide;
N-[(2Z)-3-(3-cyanophenyl)-5-methyl-1,3-thiazol-2(3H)-ylidene]-2,2-dimethylpropanamide;
N-[(2Z)-3-(2′-methyl-1,1′-biphenyl-4-yl)-1,3-thiazol-2(3H)-ylidene]pyrrolidine-1-carboxamide;
N-[(2Z)-3-(3′-methyl-1,1′-biphenyl-4-yl)-1,3-thiazol-2(3H)-ylidene]pyrrolidine-1-carboxamide;
N-[(2Z)-3-(4′-methyl-1,1′-biphenyl-4-yl)-1,3-thiazol-2(3H)-ylidene]pyrrolidine-1-carboxamide;
N-[(2Z)-3-(3′-chloro-1,1′-biphenyl-4-yl)-1,3-thiazol-2(3H)-ylidene]pyrrolidine-1-carboxamide;
N-[(2Z)-3-(4′-chloro-1,1′-biphenyl-4-yl)-1,3-thiazol-2(3H)-ylidene]pyrrolidine-1-carboxamide;
N-[(2Z)-3-(4′-methoxy-1,1′-biphenyl-4-yl)-1,3-thiazol-2(3H)-ylidene]pyrrolidine-1-carboxamide;
N-[(2Z)-3-[3′-(trifluoromethyl)-1,1′-biphenyl-4-yl]-1,3-thiazol-2(3H)-ylidene]pyrrolidine-1-carboxamide;
N-[(2Z)-3-(4-thien-3-ylphenyl)-1,3-thiazol-2(3H)-ylidene]pyrrolidine-1-carboxamide;
N-[(2Z)-3-[4′-(trifluoromethyl)-1,1′-biphenyl-4-yl]-1,3-thiazol-2(3H)-ylidene]pyrrolidine-1-carboxamide;
N-[(2Z)-3-(3′-ethoxy-1,1′-biphenyl-4-yl)-1,3-thiazol-2(3H)-ylidene]pyrrolidine-1-carboxamide;
N-[(2Z)-3-(3′-methoxy-1,1′-biphenyl-4-yl)-1,3-thiazol-2(3H)-ylidene]pyrrolidine-1-carboxamide;
N-[(2Z)-3-(3′,5′-dichloro-1,1′-biphenyl-4-yl)-1,3-thiazol-2(3H)-ylidene]pyrrolidine-1-carboxamide;
N-[(2Z)-3-(2′-chloro-1,1′-biphenyl-4-yl)-1,3-thiazol-2(3H)-ylidene]pyrrolidine-1-carboxamide;
N-[(2Z)-3-(2′-methoxy-1,1′-biphenyl-4-yl)-1,3-thiazol-2(3H)-ylidene]pyrrolidine-1-carboxamide;
N-[(2Z)-3-(2′-acetyl-1,1′-biphenyl-4-yl)-1,3-thiazol-2(3H)-ylidene]pyrrolidine-1-carboxamide;
N-[(2Z)-3-(3′-acetyl-1,1′-biphenyl-4-yl)-1,3-thiazol-2(3H)-ylidene]pyrrolidine-1-carboxamide;
N-[(2Z)-3-(4′-acetyl-1,1′-biphenyl-4-yl)-1,3-thiazol-2(3H)-ylidene]pyrrolidine-1-carboxamide;
N-[(2Z)-3-(3′,4′-dichloro-1,1′-biphenyl-4-yl)-1,3-thiazol-2(3H)-ylidene]pyrrolidine-1-carboxamide;
N-[(2Z)-3-(4-pyridin-3-ylphenyl)-1,3-thiazol-2(3H)-ylidene]pyrrolidine-1-carboxamide;
N-[(2Z)-3-(2′,5′-dichloro-1,1′-biphenyl-4-yl)-1,3-thiazol-2(3H)-ylidene]pyrrolidine-1-carboxamide;
N-[(2Z)-3-(3′-cyano-1,1′-biphenyl-4-yl)-1,3-thiazol-2(3H)-ylidene]pyrrolidine-1-carboxamide;
N-[(2Z)-3-(2′,3′-dichloro-1,1′-biphenyl-4-yl)-1,3-thiazol-2(3H)-ylidene]pyrrolidine-1-carboxamide;
N-[(2Z)-3-(2′,4′-dichloro-1,1′-biphenyl-4-yl)-1,3-thiazol-2(3H)-ylidene]pyrrolidine-1-carboxamide;
N-[(2Z)-3-(4′-cyano-1,1′-biphenyl-4-yl)-1,3-thiazol-2(3H)-ylidene]pyrrolidine-1-carboxamide;
N-[(2Z)-3-[4-(1-methyl-1H-pyrazol-4-yl)phenyl]-1,3-thiazol-2(3H)-ylidene]pyrrolidine-1-carboxamide;
N-[(2Z)-3-[4-(6-methoxypyridin-3-yl)phenyl]-1,3-thiazol-2(3H)-ylidene]pyrrolidine-1-carboxamide;
N-[(2Z)-3-(1,1′-biphenyl-4-yl)-1,3-thiazol-2(3H)-ylidene]pyrrolidine-1-carboxamide;
N-[(2Z)-3-[4-(2-methoxypyrimidin-5-yl)phenyl]-1,3-thiazol-2(3H)-ylidene]pyrrolidine-1-carboxamide;
N-[(2Z)-3-[4′-(cyanomethyl)-1,1′-biphenyl-4-yl]-1,3-thiazol-2(3H)-ylidene]pyrrolidine-1-carboxamide;
N-[(2Z)-3-quinoxalin-6-yl-1,3-thiazol-2(3H)-ylidene]pyrrolidine-1-carboxamide;
N-[(2Z)-3-(1-benzothien-5-yl)-1,3-thiazol-2(3H)-ylidene]pyrrolidine-1-carboxamide;
N-[(2Z)-3-pyrazin-2-yl-1,3-thiazol-2(3H)-ylidene]pyrrolidine-1-carboxamide;
N-[(2Z)-3-quinoxalin-2-yl-1,3-thiazol-2(3H)-ylidene]pyrrolidine-1-carboxamide;
N-[(2Z)-3-[2-(methylthio)pyrimidin-4-yl]-1,3-thiazol-2(3H)-ylidene]pyrrolidine-1-carboxamide;
N-[(2Z)-3-(6-phenylpyridazin-3-yl)-1,3-thiazol-2(3H)-ylidene]pyrrolidine-1-carboxamide;
N-[(2Z)-3-(6-methylpyridin-2-yl)-1,3-thiazol-2(3H)-ylidene]pyrrolidine-1-carboxamide;
N-[(2Z)-3-(1-benzothien-3-yl)-1,3-thiazol-2(3H)-ylidene]pyrrolidine-1-carboxamide;
N-[(2Z)-3-(4′-fluoro-1,1′-biphenyl-4-yl)-1,3-thiazol-2(3H)-ylidene]pyrrolidine-1-carboxamide;
N-[(2Z)-3-(5-fluoropyridin-2-yl)-1,3-thiazol-2(3H)-ylidene]pyrrolidine-1-carboxamide;
N-[(2Z)-3-(6-fluoropyridin-3-yl)-1,3-thiazol-2(3H)-ylidene]pyrrolidine-1-carboxamide;
N-[(2Z)-3-[5-(trifluoromethyl)pyridin-3-yl]-1,3-thiazol-2(3H)-ylidene]pyrrolidine-1-carboxamide;
N-[(2Z)-3-[5-(trifluoromethyl)pyridin-2-yl]-1,3-thiazol-2(3H)-ylidene]pyrrolidine-1-carboxamide;
N-[(2Z)-3-[4-(trifluoromethyl)pyridin-2-yl]-1,3-thiazol-2(3H)-ylidene]pyrrolidine-1-carboxamide;
N-[(2Z)-3-[6-(trifluoromethyl)pyridin-2-yl]-1,3-thiazol-2(3H)-ylidene]pyrrolidine-1-carboxamide;
N-[(2Z)-3-(6-ethoxy-2-naphthyl)-1,3-thiazol-2(3H)-ylidene]pyrrolidine-1-carboxamide;
N-[(2Z)-3-pyrido[2,3-b]pyrazin-7-yl-1,3-thiazol-2(3H)-ylidene]pyrrolidine-1-carboxamide;
N-[(2Z)-3-(4-cyclohexylphenyl)-1,3-thiazol-2(3H)-ylidene]pyrrolidine-1-carboxamide;
N-[(2Z)-3-(5-chloropyridin-2-yl)-1,3-thiazol-2(3H)-ylidene]pyrrolidine-1-carboxamide;
N-[(2Z)-3-[2-cyano-4-(trifluoromethyl)phenyl]-1,3-thiazol-2(3H)-ylidene]pyrrolidine-1-carboxamide;
N-[(2Z)-3-(5-phenylthien-2-yl)-1,3-thiazol-2(3H)-ylidene]pyrrolidine-1-carboxamide;
N-[(2Z)-3-[6-(1H-pyrazol-1-yl)pyridin-2-yl]-1,3-thiazol-2(3H)-ylidene]pyrrolidine-1-carboxamide;
N-[(2Z)-3-(6-methylpyrazin-2-yl)-1,3-thiazol-2(3H)-ylidene]pyrrolidine-1-carboxamide;
N-[(2Z)-3-(2-methylpyrimidin-4-yl)-1,3-thiazol-2(3H)-ylidene]pyrrolidine-1-carboxamide;
N-[(2Z)-3-(2,6-dimethylpyrimidin-4-yl)-1,3-thiazol-2(3H)-ylidene]pyrrolidine-1-carboxamide;
N-[(2Z)-3-(2-fluoropyridin-4-yl)-1,3-thiazol-2(3H)-ylidene]pyrrolidine-1-carboxamide;
N-[(2Z)-3-(2-methyl-1,3-benzothiazol-5-yl)-1,3-thiazol-2(3H)-ylidene]pyrrolidine-1-carboxamide;
(3R)-3-fluoro-N-[(2Z)-3-(4-isopropylphenyl)-5-methyl-1,3-thiazol-2(3H)-ylidene]pyrrolidine-1-carboxamide;
(3R)—N-[(2Z)-3-(3,4-dimethylphenyl)-5-methyl-1,3-thiazol-2(3H)-ylidene]-3-fluoropyrrolidine-1-carboxamide;
(3R)-3-fluoro-N-[(2Z)-3-(4-methoxyphenyl)-5-methyl-1,3-thiazol-2(3H)-ylidene]pyrrolidine-1-carboxamide;
(3R)-3-fluoro-N-[(2Z)-3-(4′-fluoro-1,1′-biphenyl-4-yl)-5-methyl-1,3-thiazol-2(3H)-ylidene]pyrrolidine-1-carboxamide;
2,2-dimethyl-N-[(2Z)-5-methyl-3-quinoxalin-6-yl-1,3-thiazol-2(3H)-ylidene]propanamide;
N-[(2Z)-3-(1-benzothien-5-yl)-5-methyl-1,3-thiazol-2(3H)-ylidene]-2,2-dimethylpropanamide;
2,2-dimethyl-N-[(2Z)-5-methyl-3-pyrazin-2-yl-1,3-thiazol-2(3H)-ylidene]propanamide;
2,2-dimethyl-N-[(2Z)-5-methyl-3-quinoxalin-2-yl-1,3-thiazol-2(3H)-ylidene]propanamide;
2,2-dimethyl-N-[(2Z)-5-methyl-3-[2-(methylthio)pyrimidin-4-yl]-1,3-thiazol-2(3H)-ylidene]propanamide;
2,2-dimethyl-N-[(2Z)-5-methyl-3-(6-phenylpyridazin-3-yl)-1,3-thiazol-2(3H)-ylidene]propanamide;
2,2-dimethyl-N-[(2Z)-5-methyl-3-(6-methylpyridin-2-yl)-1,3-thiazol-2(3H)-ylidene]propanamide;
N-[(2Z)-3-(1-benzothien-3-yl)-5-methyl-1,3-thiazol-2(3H)-ylidene]-2,2-dimethylpropanamide;
N-[(2Z)-3-(4′-fluoro-1,1′-biphenyl-4-yl)-5-methyl-1,3-thiazol-2(3H)-ylidene]-2,2-dimethylpropanamide;
N-[(2Z)-3-(5-fluoropyridin-2-yl)-5-methyl-1,3-thiazol-2(3H)-ylidene]-2,2-dimethylpropanamide;
N-[(2Z)-3-(6-chloroquinoxalin-2-yl)-5-methyl-1,3-thiazol-2(3H)-ylidene]-2,2-dimethylpropanamide;
2,2-dimethyl-N-[(2Z)-5-methyl-3-[5-(trifluoromethyl)pyridin-3-yl]-1,3-thiazol-2(3H)-ylidene]propanamide;
2,2-dimethyl-N-[(2Z)-5-methyl-3-[5-(trifluoromethyl)pyridin-2-yl]-1,3-thiazol-2(3H)-ylidene]propanamide;
2,2-dimethyl-N-[(2Z)-5-methyl-3-[4-(trifluoromethyl)pyridin-2-yl]-1,3-thiazol-2(3H)-ylidene]propanamide;
2,2-dimethyl-N-[(2Z)-5-methyl-3-[6-(trifluoromethyl)pyridin-2-yl]-1,3-thiazol-2(3H)-ylidene]propanamide;
2,2-dimethyl-N-[(2Z)-5-methyl-3-(6-methylpyridazin-3-yl)-1,3-thiazol-2(3H)-ylidene]propanamide;
2,2-dimethyl-N-[(2Z)-5-methyl-3-pyrido[2,3-b]pyrazin-7-yl-1,3-thiazol-2(3H)-ylidene]propanamide;
N-[(2Z)-3-(4-cyclohexylphenyl)-5-methyl-1,3-thiazol-2(3H)-ylidene]-2,2-dimethylpropanamide;
N-[(2Z)-3-(5-chloropyridin-2-yl)-5-methyl-1,3-thiazol-2(3H)-ylidene]-2,2-dimethylpropanamide;
N-[(2Z)-3-[2-cyano-4-(trifluoromethyl)phenyl]-5-methyl-1,3-thiazol-2(3H)-ylidene]-2,2-dimethylpropanamide;
2,2-dimethyl-N-[(2Z)-5-methyl-3-[2-(trifluoromethyl)pyrimidin-4-yl]-1,3-thiazol-2(3H)-ylidene]propanamide;
2,2-dimethyl-N-[(2Z)-5-methyl-3-(5-phenylthien-2-yl)-1,3-thiazol-2(3H)-ylidene]propanamide;
2,2-dimethyl-N-[(2Z)-5-methyl-3-(6-methylpyrazin-2-yl)-1,3-thiazol-2(3H)-ylidene]propanamide; and
2,2-dimethyl-N-[(2Z)-5-methyl-3-(2-methylpyrimidin-4-yl)-1,3-thiazol-2(3H)-ylidene]propanamide;
   or a pharmaceutically acceptable salt, ester, amide or prodrug thereof.

Other examples of compounds reported as positive allosteric modulators are 5-hydroxyindole analogs, for example as described in WO 01/32619, WO 01/32620, WO 01/32622; tetrahydroquinoline derivatives, for examples as described in WO 04/098600; amino-thiazole derivatives; and diarylurea derivatives, for example as described in WO 04/085433.

Compounds modulating activity of nicotinic acetylcholine receptor α7 subtype are suitable for the invention regardless of the manner in which they affect the receptor. Other compounds reported as demonstrating α7 activity include, but are not limited to, quinuclidine amide derivatives, for example PNU-282987, N-[(3R)-1-azabicyclo[2.2.2]oct-3-yl]-4-chlorobenzamide, and others as described in WO 04/052894, and MEM-3454. Additional compounds can include, but are not limited to, ARR-17779, AZD0328, WB-56203, SSR-180711A, GTS21, OH-GTS-21, and XY4083, which are all described in the publicly available literature. Yet other compounds that are reportedly under investigation that demonstrate α7 are TC-5619, ispronicline, and varenicline. Further information on TC-5619 can be obtained from Targacept. Further information on varenicline can be obtained from Pfizer.

### Histamine-3 Receptor Ligands

The activity at the H₃ receptors can be modified or regulated by the administration of H₃ receptor ligands. The ligands can demonstrate antagonist, inverse agonist, agonist, or partial agonist activity. The use of histamine-3 receptor antagonists is particularly contemplated. Examples of suitable histamine-3 receptor ligands can include, but are not limited to, aminoalkoxybiphenyl carboxamide derivatives, benzofuranyl amine derivatives, bicyclic substituted amine derivatives, di- and tri-substituted pyrrolidine derivatives, octahydropyrrolopyrrole derivatives, phenylcyclobutane derivatives, benzothiazole derivatives, azacyclosteroid derivatives, and cyclopropyl amine derivatives.

Examples of compounds reported as histamine-3 receptor ligands are well-known in the literature. For example, aminoalkoxybiphenyl carboxamide derivatives and methods for preparing them are described in WO 02/40461. Benzofuranyl amine derivatives and methods for preparing them are described in WO 02/74758. Bicyclic substituted amine derivatives and methods for preparing them are described in WO 04/43458. Di- and tri-substituted pyrrolidine derivatives and methods for preparing them are described in WO 02/06223 and WO 03/59341. Octahydropyrrolopyrrole derivatives and methods for preparing them are described in WO 2007/100990. Phenylcyclobutane derivatives and methods for preparing them are described in WO 06/132914. Benzothiazole derivatives and methods for preparing them are described in WO 07/038,074. Azacyclosteroid derivatives and methods for preparing them are described in U.S. Patent Publication No. US 2007/0066588. Cyclopropyl amine derivatives and methods for preparing them are described in U.S. Patent Publication No. 20080242653. Specific examples of compounds that are suitable histamine-3 receptor ligands include, but are not limited to, [4-(2-{2-[(2R)-2-methylpyrrolidinyl]ethyl}-benzofuran-5-yl)benzonitrile.

Other compounds demonstrating histamine-3 receptor activity that are suitable for the invention include, but are not limited to, ciproxifan; GSK-189254, which is 6-[(3-cyclobutyl-2,3,4,5-tetrahydro-1H-3-benzazepin-7-yl)oxy]-N-methyl-3-pyridinecarboxamide hydrochloride, and other benzodiazepine derivatives as described in International Publication No. WO2004/056369; GSK-239512; (1-{6-[(3-cyclobutyl-2,3,4,5-tetrahydro-1H-3-benzaaepin-7-yl)oxy]-3-pyridinyl}-2-pyrrolidinone as described in International Publication No. WO2004/056369; MK-0249; 3-(4-[1-cyclobutyl-4-piperidinyl)oxy]phenyl)-2-methyl-5-(trifluoromethyl)-4(3H)-quinazolinone, 2-methyl-3-(4-(3-(pyrrolidin-1-yl)propoxy)phenyl)-5-(trifluoromethyl)quinazolin-4(3H)-one, and other oxopyrimidine derivatives as described in U.S. Patent Publication No. US2005/0182045; carbamoyl-substituted spiro derivatives as described in International Publication No. WO2006/028239; BF-2649; (1-{3-[3-(4-chlorophenyl)propoxy]propyl}piperidine); JNJ-17216498; (4-isopropylpiperazin-1-yl)(4-(piperidin-1-ylmethyl)phenyl)methanone and (4-isopropylpiperazin-1-yl)(4-(morpholinomethyl)phenyl)methanone as described in International Publication No. WO2008/076685; PFE-03654746; 6-(2-(1-isopropylpiperidin-4-yloxy)-7,8-dihydro-1,6-naphthyridin-6(5H)-yl)nicotinamide; (1 r,3r)-N-ethyl-3-fluoro-3-(4-(3-fluoro-4-(pyrrolidin-1-ylmethyl)phenyl)cyclohexyl)cyclobutanecarboxamide; N-ethyl-3-fluoro-3-[3-fluoro-4-(pyrrolidin-1-ylmethyl)phenyl]cyclobutanecarboxamide as described in US Patent Publication No. US2008/0176925; SCH-497079; (1-((2-amino-6-methylpyridin-4-yl)methyl)-4-fluoropiperidin-4-yl)(4-(2-(pyridin-2-yl)-3H-imidazo[4,5-b]pyridin-3-yl)piperidin-1-yl)methanone; non-imidazole derivatives such as described in U.S. Pat. No. 6,720,328; CEP-26401; 6-{4-[3-(r-2-methyl-pyrrolidin-1-yl)-propoxy]-phenyl}-2h-pyridazin-3-one; compounds as described in US Patent Publication No. US2008/0027041; pyrrolidine derivatives as described in International Publication No. WO2008/137087; SAR-110894; APD916; and 4′-(2-(2-methylpyrrolidin-1-yl)ethyl)-N-(tetrahydro-2H-pyran-4-yl)biphenyl-4-sulfonamide and other pyrrolidine derivatives as described in International Publication No. WO2008/005338.

In addition to the specific compounds, one of ordinary skill in the art would readily recognize that a variety of pharmaceutically acceptable salts, esters, and amides of a parent compound also can be incorporated into a composition, method, or article of manufacture of the present invention.

Suitable pharmaceutically acceptable basic addition salts include, but are not limited to cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Other possible compounds include pharmaceutically acceptable amides and esters. “Pharmaceutically acceptable ester” refers to those esters which retain, upon hydrolysis of the ester bond, the biological effectiveness and properties of the carboxylic acid and are not biologically or otherwise undesirable. For a description of pharmaceutically acceptable esters as prodrugs, see Bundgaard, E ed., (1985) Design of Prodrugs, Elsevier Science Publishers, Amsterdam, which is hereby incorporated by reference. These esters are typically formed from the corresponding carboxylic acid and an alcohol. Generally, ester formation can be accomplished via conventional synthetic techniques. (See, e.g., March Advanced Organic Chemistry, 3rd Ed., John Wiley & Sons, New York p. 1157 (1985) and references cited therein, and Mark et al. Encyclopedia of Chemical Technology, John Wiley & Sons, New York (1980), both of which are hereby incorporated by reference. The alcohol component of the ester will generally comprise (i) a C2-C12 aliphatic alcohol that can or can not contain one or more double bonds and can or can not contain branched carbons or (ii) a C7-C12 aromatic or heteroaromatic alcohols. This invention also contemplates the use of those compositions which are both esters as described herein and at the same time are the pharmaceutically acceptable salts thereof.

“Pharmaceutically acceptable amide” refers to those amides which retain, upon hydrolysis of the amide bond, the biological effectiveness and properties of the carboxylic acid and are not biologically or otherwise undesirable. For a description of pharmaceutically acceptable amides as prodrugs, see Bundgaard, H., Ed., (1985) Design of Prodrugs, Elsevier Science Publishers, Amsterdam. These amides are typically formed from the corresponding carboxylic acid and an amine. Generally, amide formation can be accomplished via conventional synthetic techniques. (See, e.g., March Advanced Organic Chemistry, 3rd Ed., John Wiley & Sons, New York, p. 1152 (1985) and Mark et al. Encyclopedia of Chemical Technology, John Wiley & Sons, New York (1980), both of which are hereby incorporated by reference. This invention also contemplates the use of those compositions which are amides, as described herein, and at the same time are the pharmaceutically acceptable salts thereof.

It also will be readily apparent to one with skill in the art that the compounds can be generated in vivo by administration of a drug precursor which, following administration, releases the drug in vivo via a chemical or physiological process (e.g., a parent compound on being brought to the physiological pH or through enzyme action is converted to the desired drug form).

### Psychostimulants

Psychostimulants are well known for ADHD therapy. Their dosage and administration are publicly available in the product literature and other publicly available sources. However, for the sake of illustration a few non-limiting examples of psychostimulants are particularly suitable for the invention, such as methylphenidate, dextroamphetamine, amphetamine, and pemoline. Examples of suitable dosages of such active agents are further described below in connection with the Administration of compositions and methods of the invention.

Psychostimulants also include stimulant-like compounds that can increase alertness and awareness. Examples of such compounds include, but are not limited to, modafinil (or modaphonil), armodafinil, and the like.

### Monoamine Reuptake Inhibitors

Monoamine Reuptake Inhibitors are well known for ADHD therapy. Their dosage and administration are publicly available in the product literature and other publicly available sources. However, for the sake of illustration a few non-limiting examples of monoamine reuptake inhibitors are particularly suitable for the invention, such as desipramine, notriptyline, atomoxetine (or tomoxetine), reboxetine, venlafaxine, citalopram, escitalopram, fluoxetine, and bupropion. Examples of suitable dosages of such active agents are further described below in connection with the Administration of compositions and methods of the invention.

### Administration

As noted above, it has been discovered that cognitive conditions can be treated by concurrently administering to a patient (i.e. a human) in need thereof, a nicotinic acetylcholine receptor ligand and a histamine-3 receptor ligand. Similarly, the nicotinic acetylcholine receptor ligand or histamine-3 receptor ligand each can be concurrently administered with a psychostimulant or a monoamine reuptake inhibitor.

As used in this application, the term “concurrent administration” refers to administering the nicotinic receptor ligand to a patient, who has been prescribed (or has consumed) at least one histamine-3 receptor ligand, or other active agent for treating attention-deficit hyperactivity disorder, such as psychostimulants or monoamine reuptake inhibitors, at an appropriate time so that the patient's symptoms may subside. This may mean simultaneous administration of the active agents, or administration of the medications at different, but appropriate times. Establishing such a proper dosing schedule will be readily apparent to one skilled in the art, such as a psychiatrist, or other physician.

The dosage range at which the active agents will be administered concurrently can vary widely. The specific dosage will be chosen by the patient's physician taking into account the particular medicament chosen, the severity of the patient's illness, any other medical conditions or diseases the patient is suffering from, other drugs the patient is taking and their potential to cause an interaction or adverse event, the patient's previous response to medication, and other factors.

The active agents should be administered concurrently in amounts that are effective to treat the patient's attention-deficit hyperactivity disorder, schizophrenia or related condition. In more general terms, one would create a combination of the present invention by choosing a dosage of a nicotinic receptor ligand and a histamine-3 receptor ligand, psychostimulant, or monoamine reuptake inhibitor, according to the spirit of the guidelines presented above.

The cognitive therapy of the present invention is carried out by administering a nicotinic receptor ligand and a histamine-3 receptor ligand, psychostimulant, or monoamine reuptake inhibitor, in any manner which provides effective levels of the compounds in the body at the same time. Typically, the combination will be administered orally.

However, the invention is not limited to oral administration. The invention should be construed to cover any route of administration that is appropriate for the medications involved and for the patient. For example, transdermal administration may be very desirable for patients who are forgetful or petulant about taking oral medicine. Injections may be appropriate for patients refusing their medication. One of the drugs may be administered by one route, such as oral, and the others may be administered by the transdermal, percutaneous, intravenous, intramuscular, intranasal, or intrarectal route, in particular circumstances. The route of administration may be varied in any way, limited by the physical properties of the drugs and the convenience of the patient and the caregiver.

The following examples are being presented to further illustrate the invention. They should not be construed as limiting the invention in any manner. The dosage range of the currently available psychostimulants and monoamine reuptake inhibitors can be broad. Therefore, as an example, typical dose ranges for some commonly used antipsychotics are below. This list is not intended to be complete but is merely an illustration of current clinical usage.

TABLE 1
Active Agents for ADHD Treatment

| Active Agent | Clinical Dose Range |
|---|---|
| Methylphenidate | 0.01 to 0.85 mg/kg per day |
| (RITALIN) | |
| Dextroamphetamine | 0.07 to 0.85 mg/kg per day |
| (DEXEDRINE) | |
| Amphetamine | 0.05 to 0.6 mg/kg per day |
| (ADDERALL) | |
| Pemoline (CYLERT) | 0.1 to 1.6 mg/kg per day |
| Desipramine | 0.5 to 5.0 mg/kg per day |
| (NORPRAMIN) | |
| Nortriptyline | 0.1 to 3.0 mg/kg per day |
| Atomoxetine | 0.1 to 3.0 mg/kg per day or |
| (STRATTERA) | 0.1 to 1.1 mg/kg per day |
| Reboxetine | 0.03 to 3.0 mg/kg per day |
| Fluoxetine | 0.2 to 20 mg/kg per day |
| (PROZAC) | |
| Bupropion | 1.0 to 4.3 mg/kg per day |
| (WELLBUTRIN) | |
| Modafinil | 1.0 to 5.7 mg/kg per day |
| (PROVIGIL) | |
| Armodafinil | 1.0 to 5.7 mg/kg per day |
| (NUVIGIL) | |

The term “effective amount” as used herein refers to a sufficient amount of the individual compound to treat or prevent anxiety disorders, mood disorders, and psychotic disorders or the condition to be treated at a reasonable benefit/risk ratio in the judgment of the administering specialist applicable to any medical treatment.

The term “sub efficacious” as used herein, for example to refer to a “sub efficacious dose” or a “sub efficacious amount” refers to a dose or amount of the individual compound less than an amount for treating or preventing anxiety disorders, mood disorders, psychotic disorders or the condition to be treated at a reasonable benefit/risk ratio in the judgment of the administering specialist applicable to the medical treatment.

The term “maximally efficacious” as used herein, for example to refer to a “maximally efficacious dose” or a “maximally efficacious amount” refers to a dose or amount of the individual compound having the greatest effect for treating or preventing anxiety disorders, mood disorders, psychotic disorders or the condition to be treated at a reasonable benefit/risk ratio in the judgment of the administering specialist applicable to the medical treatment.

The specific effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The exact formulation, route of administration, and dosage can be chosen by the individual physician in view of the patient's condition. Dosage amount and interval can be adjusted individually to provide plasma levels of the active moiety which are sufficient to maintain therapeutic effects.

The following dosage amounts and other dosage amounts set forth elsewhere in this description and in the appendant claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and throughout the appendant claims, if applicable, are daily doses.

The suitable amount of active agent is based on recommended dose range, preferably at the low end, for example as illustrated in Table 1, and combined with an effective dose of a nicotinic acetylcholine receptor ligand or a histamine-3 receptor ligand. The effective dose range of the nicotinic acetylcholine receptor ligand or histamine-3 receptor ligand will be adjusted to ensure efficacious plasma levels judged from clinical trials and can range depending on the duration of administration (once or twice daily or sustained release) of the product, as recommended by the manufacturer.

### Formulations

The active agents can be administered as a single pharmaceutical composition, or separately to achieve a concomitant or controlled effect. Such compositions may take any physical form that is suitable for pharmaceuticals. Pharmaceutical compositions suitable for oral administration are particularly preferred. Such pharmaceutical compositions contain an effective amount of each of the compounds, which effective amount is related to the daily dose of the compounds to be administered. Each dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the antipsychotic therapy is being given.

The composition contains at least one pharmaceutically acceptable excipient, or inert ingredient. The inert ingredients and manner of formulating the pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The specific combination of any active agents can be chosen and formulated solely for convenience and economy. Any of the combinations may be formulated in any desired form of composition. Some examples of compositions are described herein, followed by some typical formulations.

Capsules are prepared by mixing the compounds with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours, and similar edible powders.

If desired, the capsules can be formulated so that the contents are removed from the capsules prior to ingestion by the patient. The capsule contents may be diluted in foods, juices, or other substance, in order to simplify administration to those who have difficulty swallowing. Methods for manufacturing such a dosage form would be readily apparent to one skilled in the art.

The medications may also be formulated into liquids or syrups, as is known in the art, in order to simplify administration. The medication can be dissolved in or added to liquids, flavorants, antioxidants, stabilizers, or other inactive ingredients, as is known in the art. Such dosage forms have particular suitability with the elderly, such as dementia patients.

Tablets are prepared by direct compression, by wet granulation, or by dry granulation. Their formulations usually incorporate diluents, binders, lubricants, and disintegrators as well as the compound. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid, and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp, and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches also are suitable for administering the combination. Typically transdermal patches comprise a resinous composition in which the drugs will dissolve, or partially dissolve, which is held in contact with the skin by a film which protects the composition. More complicated patch compositions are also in use, particularly those having a membrane pierced with innumerable pores through which the drugs are pumped by osmotic action.

### Packaging

To enhance patient convenience, any combination may be formulated into a single dosage form. Alternatively, separate dosage forms can be used, yet packaged in a single container for dispensing by the pharmacist, for example, as with a blister pack. Such packaging is typically designed to help a patient comply with a dosage regimen and to consume all of the required medication.

An article of manufacture, typically refers to the packaging, can be a first pharmaceutical dosage form with a nicotinic acetylcholine receptor ligand and a second pharmaceutical dosage form with histamine-3 receptor ligand. Alternatively, the article of manufacture can be a first pharmaceutical dosage form with a nicotinic acetylcholine receptor ligand (preferably of α4β2 subtype) or a histamine-3 receptor ligand with a second pharmaceutical dosage form of a psychostimulant or a monoamine reuptake inhibitor. The article of manufacture can contain a first and second pharmaceutical dosage form in a single dosage form or as separate dosage forms.

Examples of such packaging are well known to those skilled in the pharmaceutical arts. For example, Pfizer distributes an antibiotic known as Zithromax®. Patients must consume 2 pills on the first day and one pill after that for 4 days in order to eradicate the infection. To allow a patient to comply with such a complicated schedule, Pfizer packages the medication in a blister pack that is commonly referred to as a Z-pack. Similar packages are used with steroids in which the dosage must be tapered. Birth control pills are another example of packaging pharmaceuticals to enhance convenience.

The composition may be incorporated into such packaging to enhance patient convenience. If desired, such packaging may be used even if the active agents are in a single dosage form. The particulars of such packaging will be readily apparent to one skilled in the art.

As is well-known to those skilled in the art, the packaged pharmaceutical will include an insert. Such insert describes the drugs, their doses, possible side effects and indication.

The compounds may be in a single or separate dosage forms.

The compositions, methods, and articles of manufacture have been described with reference to various specific embodiments and techniques. The examples described herein illustrate but do not limit the scope of the invention as defined in the appended claims and equivalents thereof.

### EXAMPLES

### Example 1

### The Effect of Atomoxetine in 5-trial Inhibitory Avoidance in the Spontaneously Hyperactive rat pup

Methods: In this behavioral paradigm, hypothesized to test aspects of both attention and impulsivity, vehicle-treated Spontaneously Hypertensive rat (SHR) pups show an impaired ability to withhold a natural response to transfer from the light into a preferred dark chamber that is paired with a mild footshock. In this study, SHR pups at postnatal day 22-23 were dosed with either vehicle or atomoxetine (0.001, 0.01, 0.1, 1.0 and 6.0 mg/kg, s.c.) 30 min prior to being tested. They were then placed into the brightly illuminated compartment of a two compartment chamber (Hamilton Kinder, San Diego, Calif.) with a retractable door between the light and dark compartments opened. After animals transferred through the open door to the dark compartment, the door closed automatically and a 0.1 mA scrambled current was applied to a 31 bar stainless steel grid floor for one second. At this point, the pup was removed and returned to its home cage and littermates for a one minute inter trial interval, and the transfer latency was noted. The same pup was returned to the bright compartment, and the process repeated for five trials. Latency to enter the dark compartment was used to evaluate learning.

### Results:

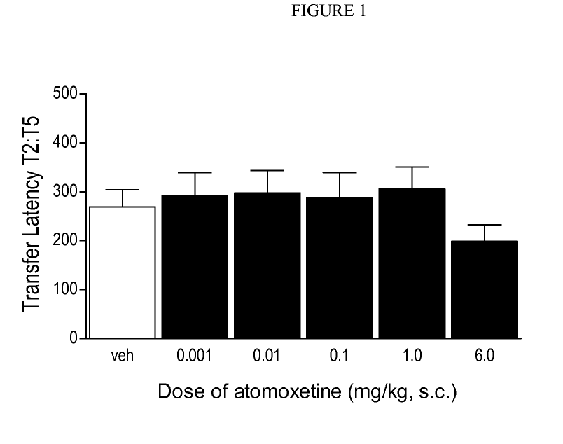

As shown in FIG. 1, atomoxetine did not alter transfer latency compared to vehicle treated animals (one way ANOVA: F(5,73)=0.58). This indicates that atomoxetine did not show efficacy in the SHR 5-trial inhibitory avoidance within the dose range tested.

### Example 2

### The Effect of the alpha7 Ligand, Compound 1, in 5-Trial Inhibitory Avoidance in the Spontaneously Hyperactive rat pup

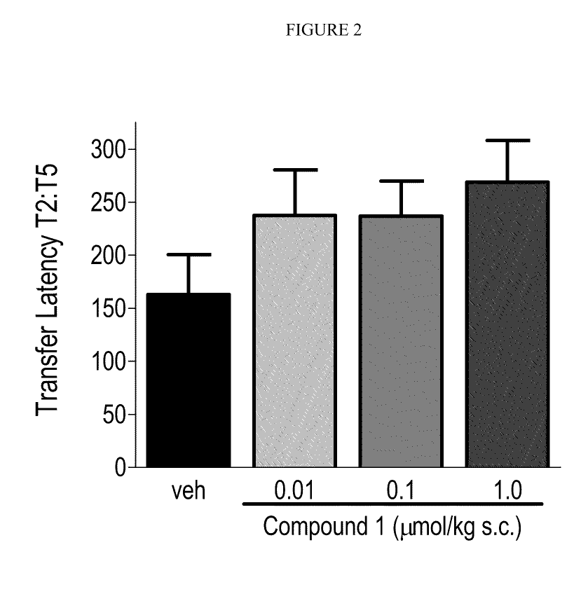

Methods: In this behavioral paradigm, hypothesized to test aspects of both attention and impulsivity, vehicle-treated SHR rat pups show an impaired ability to withhold a natural response to transfer from the light into a preferred dark chamber that is paired with a mild footshock. In this study, SHR pups at postnatal day 21-25 were dosed with either vehicle or the alpha7 agonist, compound 1,2-methyl-5-[6-phenylpyridazin-3-yl]octahydropyrrolo[3,4-c]pyrrole, (0.01, 0.1, and 1.0 umol/kg, s.c.) 30 min prior to being tested. They were then placed into the brightly illuminated compartment of a two compartment chamber (Hamilton Kinder, San Diego, Calif.) with a retractable door between the light and dark compartments opened. After animals transferred through the open door to the dark compartment, the door closed automatically and a 0.1 mA scrambled current was applied to a 31 bar stainless steel grid floor for one second. At this point, the pup was removed and returned to its home cage and littermates for a one minute inter trial interval, and the transfer latency was noted. The same pup was returned to the bright compartment, and the process repeated for five trials. Latency to enter the dark compartment was used to evaluate learning. Methods for evaluating 2-methyl-5-[6-phenylpyridazin-3-yl]octahydropyrrolo[3,4-c]pyrrole also are described in Tietje, Karin R., et al., Preclinical Characterization of A-582941: A Novel α7 Neuronal Nicotinic Receptor Agonist with Broad Spectrum Cognition-Enhancing Properties, CNS Neuroscience & Therapeutics 14 (2008) 65-82 (2008).
Results: As shown in FIG. 2, Compound 1 induced a significant, dose dependent effect (Mann Whitney, p<0.05), with the dose of 1 μmol/kg significantly increasing transfer latency. The level of efficacy was similar to that previously seen with methylphenidate (Fox et al., Behav Brain Res, 131 (2002): 151-161).

The 5-trial inhibitory avoidance assay in spontaneously hypertensive (SHR) rat pups has been proposed as a model with predictive validity for compounds showing efficacy in attention disorders such as ADHD. Stimulants such as methylphenidate are fully efficacious in this model (Fox et al., Behav Brain Res, 131 (2002): 151-161). In contrast, atomoxetine is not efficacious in the SHR 5-trial inhibitory avoidance model as is illustrated in Example 1. Thus, this model is able to differentiate medications that are fully efficacious in the clinic from those that only show moderate efficacy.

Similar to methylphenidate, alpha4beta2 ligands such ABT-418 and ABT-089 show full efficacy in SHR 5-trial inhibitory avoidance (Fox et al., Behav Brain Res, 131 (2002): 151-161; Decker et al, Society for Neuroscience, 2008). As can be seen in Example 1, alpha7 agonists such as Compound 1 also show full efficacy in this model. In addition, as reported by Rezvani, et al. in Rezvani, Amir H., Kholdebarin, Ehsan, Brucato, Frederic, Callahan, Patrick M., Lowe, David A., Levin, Edward D., Effect of R3487/MEM3454, a novel nicotinic α7 receptor partial agonist and 5-HT3 antagonist on sustained attentionbin rats, Progress in Neuropsychopharmacology & Biological Psychiatry (2008), doi: 10.1016/j.pnpbp.2008.11.018, alpha7 receptor partial agonists, for example R3487/MEM3454, improve visual sustained attention in rats performing an operant visual signal detection task. Histamine H₃ antagonists, such as ABT-239, show full efficacy in SHR 5-trial inhibitory avoidance (Fox et al., J Pharmacol Exp Ther, 313 (2005): 176-190). These results are summarized in the table below. Together, these data indicate that adding treatment with alpha4beta2, alpha7 or H₃ receptor ligands to treatment with reuptake inhibitors, such as atomoxetine, will result in greater clinical efficacy than that seen with reuptake inhibitors given alone.

| | | Level of Efficacy in | |
|---|---|---|---|
| | Example | SHR 5-trial Inhibitory | |
| Compound Class | Compounds | Avoidance Model | Reference |
| Stimulant | Methylphenidate | Full | Fox et al., Behav Brain |
| | | | Res, 131 (2002): 151-161 |
| Reuptake inhibitors | Atomoxetine | None | Example 1 |
| NNR alpha4beta2 | ABT-089, ABT-418 | Full | Fox et al., Behav Brain |
| ligands | | | Res, 131 (2002): 151-161; |
| | | | Decker et al, Society for |
| | | | Neuroscience, 2008 |
| NNR alpha7 ligands | Compound 1 | Full | Example 2 |
| H3 antagonists | ABT-239 | Full | Fox et al., J Pharmacol |
| | | | Exp Ther, 313 (2005): |
| | | | 176-190 |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the compounds, chemical structures, substituents, derivatives, dosages, formulations, or methods, or any combination of such changes and modifications of use of the invention, may be made without departing from the spirit and scope thereof.

What is claimed is:

1. A composition, comprising:
(i) a nicotinic ligand, wherein the nicotinic ligand is a nicotinic subtype α4β2 receptor ligand or a nicotinic subtype α7 receptor ligand; and
(ii) a histamine-3 receptor ligand.

2. The composition of claim 1, wherein the nicotinic subtype α4β2 receptor ligand is a nicotinic subtype α4β2 agonist or a nicotinic subtype α4β2 partial agonist.

3. The composition of claim 1, wherein the nicotinic subtype α4β2 receptor ligand is selected from a heterocyclic ether derivative, a N-substituted diazabicyclic derivative, or a heterocyclic substituted amino azacycle compound.

4. The composition of claim 1, wherein the nicotinic subtype α4β2 receptor ligand is selected from:
5-[(2R)-azetidin-2-ylmethoxy]-2-chloropyridine;
(3R)-1-pyridin-3-ylpyrrolidin-3-amine;
2-methyl-3-(2-(S)-pyrrolidinylmethoxy)pyridine;
3-(5,6-dichloro-pyridin-3-yl)-1S,5S-3,6-diazabicyclo[3.2.0]heptane;
(R,R)-1-(pyridin-3-yl)octahydro-pyrrolo[3,4-b]pyrrole;
6,1,0-methano-6H-pyrazino[2,3-h][3]benzazepine;
7,8,9,10-tetrahydro-(2S,4E)-N-methyl-5-(5-isopropoxy-3-pyridyl)-4-penten-2-amine;
(2S,4E)-N-methyl-5-(5-methoxy-3-pyridyl)-4-penten-2-amine;
(2S,4E)-N-methyl-5-(5-ethoxy-3-pyridyl)-4-penten-2-amine;
(2S,4E)-N-methyl-3-pyrimidine-4-penten-2-amine;
(5aS,8S,10aR)-5a,6,9,10-tetrahydro-7H,11H-8,10a-methanopyrido[2′,3′:5,6]pyrano[2,3-d]azepine;
3-[1-(2,4-dimethoxy-phenyl)-meth-(E)-ylidene]-3,4,5,6-tetrahydro-[2,3′]bipyridinyl; and
3-[1-(2-methoxy-4-hydroxyphenyl)-meth-(E)-ylidene]-3,4,5,6-tetrahydro-[2,3′]bipyridinyl;
or pharmaceutically acceptable salts thereof.

5. The composition of claim 1, wherein the nicotinic subtype α7 receptor ligand is a nicotinic subtype α7 agonist, nicotinic subtype α7 partial agonist, or nicotinic subtype α7 allosteric modulator.

6. The composition of claim 1, wherein the nicotinic receptor α7 receptor ligand demonstrates a ratio of the Kᵢ value that as measured by [³H]-cytisine binding assay (Kᵢ Cyt) to the Kᵢ value as measured by MLA binding assay (Kᵢ MLA) in a formula D=Kᵢ Cyt/Kᵢ MLA such that D is greater than a value of 50.

7. The composition of claim 1, wherein the nicotinic subtype α7 receptor ligand is selected from diazabicycloalkane derivatives, spirocyclic quinuclidinic ether derivatives, bicycloheterocycle substituted quinuclidine derivatives, 3-quinuclidinyl amino-substituted biaryl derivatives, 3-quinuclidinyl heteroatom-bridged biaryl derivatives, and amino-substituted tricyclic derivatives.

8. The composition of claim 1, wherein the nicotinic subtype α7 receptor ligand is selected from:
5-(6-[(3R)-1-azabicyclo[2.2.2]oct-3-yloxy]pyridazin-3-yl)-1H-indole;
2-(6-phenylpyridazine-3-yl)octahydropyrrolo[3,4-c]pyrrole;
5-[5-{(1R,5R)-6-methyl-3,6-diaza-bicyclo[3.2.0]hept-3-yl}-pyridin-2-yl]-1H-indole;
5-[6-(cis-5-methyl-hexahydro-pyrrolo[3,4-c]pyrrol-2-yl)-pyridazin-3-yl-1H-indole; and
(1R,4R,5S)-4-(5-phenyl-[1,3,4]thiazdiazol-2-yloxy)-1-aza-tricyclo[3.3.1^{3,7}]decane;
or is a pharmaceutically acceptable salt thereof.

9. The composition of claim 1, wherein the nicotinic subtype α7 receptor ligand is a compound of formula (I): 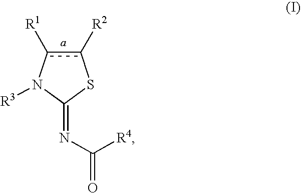
wherein
R¹ is hydrogen, methyl, phenyl, pyrazolyl, or hydroxyl;
R² is hydrogen, alkyl, alkenyl, ═CH₂, or ═CHR^{c} wherein the alkyl group and the alkenyl group are substituted with 0, 1, 2, or 3 substituents independently selected from the group consisting of alkoxycarbonyl, alkylcarbonyloxy, aryl, aryloxy, arylalkoxy, carboxy, cyano, cycloalkyl, haloalkoxy, heteroaryl, heterocycle, hydroxyl, nitro, and R^{d}R^{e}N—, wherein a group represented by R² can be further substituted with 0, 1, or 2 groups selected from halo and alkoxy;
R^{c} is alkyl or halo;
R^{d} and R^{e} are each independently hydrogen, alkyl, alkoxyalkyl, aryl, arylalkyl, cyanoalkyl, heteroaryl, heteroarylalkyl, heterocycle, or heterocyclealkyl;
R³ is optionally substituted aryl, heteroaryl, or -G¹-L-G²;
G¹ is aryl or heteroaryl;
G² is aryl, cycloalkyl, heteroaryl, or heterocycle;
L is a bond, 0, alkylene, or —O-alkylene-;
R⁴ is optionally substituted alkyl, cycloalkyl, heteroaryl, heterocycle or —NR⁵R⁶;
wherein, R⁵ and R⁶ are independently hydrogen, alkyl, (NRⁱR^{j})alkyl, alkynyl, aryl, heterocyclealkyl, cycloalkylalkyl, cyanoalkyl, cycloalkyl, alkoxyalkyl, arylalkyl, or heteroarylalkyl; and
a is single or double bond;
provided that when R¹ is hydroxyl or when R² is a radical attached to the thiazole ring through an exocyclic double bond, then a is single bond; and
provided that when a is double bond, R¹ is hydrogen, R² is methyl, R³ is phenyl substituted with 3-haloalkyl, 3-halo, 3-haloalkoxy, 2,5-dihalo, 2,3-dihalo, 3,4-dihalo, or 3,5-dihalo, then R⁴ is heterocycle;
or a pharmaceutically acceptable salt, ester, amide or prodrug thereof.

10. The composition of claim 1, wherein the nicotinic subtype α7 receptor ligand is a selected from:
N′-[(2Z)-3-(4-methoxyphenyl)-5-methyl-1,3-thiazol-2(3H)-ylidene]-N,N-dimethylurea;
N-[(2Z)-3-[4-(difluoromethoxy)phenyl]-5-methyl-1,3-thiazol-2(3H)-ylidene]pyrrolidine-1-carboxamide;
N-[(2Z)-3-[4-(difluoromethoxy)phenyl]-5-methyl-1,3-thiazol-2(3H)-ylidene]piperidine-1-carboxamide;
N′-[(2Z)-3-[4-(difluoromethoxy)phenyl]-5-methyl-1,3-thiazol-2(3H)-ylidene]-N,N-diethylurea;
N-[(2Z)-5-methyl-3-[4-(trifluoromethoxy)phenyl]-1,3-thiazol-2(3H)-ylidene]pyrrolidine-1-carboxamide;
N,N-diethyl-N′-[(2Z)-5-methyl-3-[4-(trifluoromethoxy)phenyl]-1,3-thiazol-2(3H)-ylidene]urea;
N,N-dimethyl-N′-[(2Z)-5-methyl-3-[4-(trifluoromethoxy)phenyl]-1,3-thiazol-2(3H)-ylidene]urea;
N-[(2Z)-3-(4-chlorophenyl)-5-methyl-1,3-thiazol-2(3H)-ylidene]pyrrolidine-1-carboxamide;
N-[(2Z)-3-(4-chlorophenyl)-5-methyl-1,3-thiazol-2(3H)-ylidene]piperidine-1-carboxamide;
N′-[(2Z)-3-(4-chlorophenyl)-5-methyl-1,3-thiazol-2(3H)-ylidene]-N,N-diethylurea;
N′-[(2Z)-3-(4-chlorophenyl)-5-methyl-1,3-thiazol-2(3H)-ylidene]-N-methyl-N-phenylurea;
N′-[(2Z)-3-(4-chlorophenyl)-5-methyl-1,3-thiazol-2(3H)-ylidene]-N,N-dimethylurea;
N-[(2Z)-3-(4-fluorophenyl)-5-methyl-1,3-thiazol-2(3H)-ylidene]pyrrolidine-1-carboxamide;
N-[(2Z)-3-(4-fluorophenyl)-5-methyl-1,3-thiazol-2(3H)-ylidene]piperidine-1-carboxamide;
N,N-diethyl-N′-[(2Z)-3-(4-fluorophenyl)-5-methyl-1,3-thiazol-2(3H)-ylidene]urea;
N′-[(2Z)-3-(4-fluorophenyl)-5-methyl-1,3-thiazol-2(3H)-ylidene]-N,N-dimethylurea;
N-[(2Z)-3-(4-fluorophenyl)-5-methyl-1,3-thiazol-2(3H)-ylidene]cyclobutanecarboxamide;
N-[(2Z)-5-methyl-3-[4-(trifluoromethyl)phenyl]-1,3-thiazol-2(3H)-ylidene]pyrrolidine-1-carboxamide;
N,N-dimethyl-N′-[(2Z)-5-methyl-3-[4-(trifluoromethyl)phenyl]-1,3-thiazol-2(3H)-ylidene]urea;
2-methyl-N-[(2Z)-5-methyl-3-[4-(trifluoromethyl)phenyl]-1,3-thiazol-2(3H)-ylidene]pyrrolidine-1-carboxamide;
(2R)-2-methyl-N-[(2Z)-5-methyl-3-[4-(trifluoromethyl)phenyl]-1,3-thiazol-2(3H)-ylidene]pyrrolidine-1-carboxamide;
(2S)-2-methyl-N-[(2Z)-5-methyl-3-[4-(trifluoromethyl)phenyl]-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]-2,5-dihydro-1H-pyrrole-1-carboxamide;
N-[(2Z)-3-(4-chlorophenyl)-1,3-thiazolidin-2-ylidene]pyrrolidine-1-carboxamide;
(3R)—N-[(2Z)-3-(4-chlorophenyl)-1,3-thiazolidin-2-ylidene]-3-fluoropyrrolidine-1-carboxamide;
N′-[(2Z)-3-(4-chlorophenyl)-1,3-thiazolidin-2-ylidene]-N-methyl-N-prop-2-ynylurea;
N′-[(2Z)-3-(4-chlorophenyl)-1,3-thiazolidin-2-ylidene]-N-(3-fluorobenzyl)-N-methylurea;
N-{(2Z)-5-methylene-3-[4-(trifluoromethyl)phenyl]-1,3-thiazolidin-2-ylidene}azetidine-1-carboxamide;
N-[(2Z)-5-methyl-3-[4-(trifluoromethyl)phenyl]-1,3-thiazol-2(3H)-ylidene]azetidine-1-carboxamide;
N-[(2Z)-5-methyl-3-(2-naphthyl)-1,3-thiazol-2(3H)-ylidene]pyrrolidine-1-carboxamide;
N-[(2Z)-5-methyl-3-(3-methylisothiazol-5-yl)-1,3-thiazol-2(3H)-ylidene]pyrrolidine-1-carboxamide;
N-[(2Z)-3-(4-bromophenyl)-1,3-thiazol-2(3H)-ylidene]pyrrolidine-1-carboxamide;
N-(3-fluorobenzyl)-N-methyl-N′-[(2Z)-5-methyl-3-(2-naphthyl)-1,3-thiazol-2(3H)-ylidene]urea;
(3R)—N-[(2Z)-3-(5-cyanothien-2-yl)-5-methyl-1,3-thiazol-2(3H)-ylidene]-3-fluoropyrrolidine-1-carboxamide;
N-[(2Z)-5-(hydroxymethyl)-3-(2-naphthyl)-1,3-thiazol-2(3H)-ylidene]pyrrolidine-1-carboxamide;
(3R)-3-fluoro-N-[(2Z)-5-(hydroxymethyl)-3-(2-naphthyl)-1,3-thiazol-2(3H)-ylidene]pyrrolidine-1-carboxamide;
(3R)-3-fluoro-N-[(2Z)-5-(hydroxymethyl)-3-[4-(trifluoromethyl)phenyl]-1,3-thiazol-2(3H)-ylidene]pyrrolidine-1-carboxamide;
(3R)-3-fluoro-N-[(2Z)-5-(hydroxymethyl)-3-[5-(trifluoromethyl)thien-2-yl]-1,3-thiazol-2(3H)-ylidene]pyrrolidine-1-carboxamide;
(3R)—N-[(2Z)-3-(5-chlorothien-2-yl)-5-(hydroxymethyl)-1,3-thiazol-2(3H)-ylidene]-3-fluoropyrrolidine-1-carboxamide;
(3R)—N-[(2Z)-3-(1-benzothien-5-yl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-(2,2,4,4-tetrafluoro-4H-1,3-benzodioxin-6-yl)-1,3-thiazol-2(3H)-ylidene]pyrrolidine-1-carboxamide;
(3R)—N-[(2Z)-3-(5,6-difluoro-2-naphthyl)-5-(hydroxymethyl)-1,3-thiazol-2(3H)-ylidene]-3-fluoropyrrolidine-1-carboxamide;
N-[(2Z)-5-methyl-3-phenyl-1,3-thiazol-2(3H)-ylidene]pyrrolidine-1-carboxamide;
N-[(2Z)-5-methyl-3-(3-methylphenyl)-1,3-thiazol-2(3H)-ylidene]pyrrolidine-1-carboxamide;
N-[(2Z)-5-methyl-3-(4-methylphenyl)-1,3-thiazol-2(3H)-ylidene]pyrrolidine-1-carboxamide;
N-[(2Z)-3-(3-aminophenyl)-5-methyl-1,3-thiazol-2(3H)-ylidene]pyrrolidine-1-carboxamide;
N-[(2Z)-3-(3-hydroxyphenyl)-5-methyl-1,3-thiazol-2(3H)-ylidene]pyrrolidine-1-carboxamide;
N-[(2Z)-3-(4-methoxyphenyl)-5-methyl-1,3-thiazol-2(3H)-ylidene]pyrrolidine-1-carboxamide;
N-[(2Z)-3-(3-fluorophenyl)-5-methyl-1,3-thiazol-2(3H)-ylidene]pyrrolidine-1-carboxamide;
N-[(2Z)-3-(3-chlorophenyl)-5-methyl-1,3-thiazol-2(3H)-ylidene]pyrrolidine-1-carboxamide;
N-[(2Z)-3-(2-bromophenyl)-5-methyl-1,3-thiazol-2(3H)-ylidene]pyrrolidine-1-carboxamide;
N-[(2Z)-3-(3-bromophenyl)-5-methyl-1,3-thiazol-2(3H)-ylidene]pyrrolidine-1-carboxamide;
N-[(2Z)-3-(3-cyanophenyl)-5-methyl-1,3-thiazol-2(3H)-ylidene]pyrrolidine-1-carboxamide;
N-[(2Z)-3-(4-cyanophenyl)-5-methyl-1,3-thiazol-2(3H)-ylidene]pyrrolidine-1-carboxamide;
N-[(2Z)-3-[4-(dimethylamino)phenyl]-5-methyl-1,3-thiazol-2(3H)-ylidene]pyrrolidine-1-carboxamide;
N-[(2Z)-5-methyl-3-[3-(trifluoromethyl)phenyl]-1,3-thiazol-2(3H)-ylidene]pyrrolidine-1-carboxamide;
N-[(2Z)-5-methyl-3-[3-(trifluoromethoxy)phenyl]-1,3-thiazol-2(3H)-ylidene]pyrrolidine-1-carboxamide;
N-[(2Z)-5-methyl-3-(4-phenoxyphenyl)-1,3-thiazol-2(3H)-ylidene]pyrrolidine-1-carboxamide;
N-[(2Z)-3-[4-(benzyloxy)phenyl]-5-methyl-1,3-thiazol-2(3H)-ylidene]pyrrolidine-1-carboxamide;
N-[(2Z)-3-(3,4-dimethylphenyl)-5-methyl-1,3-thiazol-2(3H)-ylidene]pyrrolidine-1-carboxamide;
N-[(2Z)-3-(3,5-dimethylphenyl)-5-methyl-1,3-thiazol-2(3H)-ylidene]pyrrolidine-1-carboxamide;
N-[(2Z)-5-methyl-3-pyridin-2-yl-1,3-thiazol-2(3H)-ylidene]pyrrolidine-1-carboxamide;
N-[(2Z)-3-(3,4-dichlorophenyl)-5-methyl-1,3-thiazol-2(3H)-ylidene]pyrrolidine-1-carboxamide;
N-[(2Z)-3-(3,5-dichlorophenyl)-5-methyl-1,3-thiazol-2(3H)-ylidene]pyrrolidine-1-carboxamide;
N-[(2Z)-5-methyl-3-[4-(methylthio)phenyl]-1,3-thiazol-2(3H)-ylidene]pyrrolidine-1-carboxamide;
N-[(2Z)-3-(3-methoxyphenyl)-5-methyl-1,3-thiazol-2(3H)-ylidene]pyrrolidine-1-carboxamide;
N-[(2Z)-3-(4-chloro-3-methylphenyl)-5-methyl-1,3-thiazol-2(3H)-ylidene]pyrrolidine-1-carboxamide;
N-[(2Z)-3-(4-benzylphenyl)-5-methyl-1,3-thiazol-2(3H)-ylidene]pyrrolidine-1-carboxamide;
N-[(2Z)-3-(1-benzothien-5-yl)-5-methyl-1,3-thiazol-2(3H)-ylidene]pyrrolidine-1-carboxamide;
N-[(2Z)-5-methyl-3-(5-methylthien-2-yl)-1,3-thiazol-2(3H)-ylidene]pyrrolidine-1-carboxamide;
N-[(2Z)-5-methyl-3-(2-methyl-1,3-benzothiazol-5-yl)-1,3-thiazol-2(3H)-ylidene]pyrrolidine-1-carboxamide;
N-[(2Z)-5-methyl-3-[6-(trifluoromethyl)pyridin-3-yl]-1,3-thiazol-2(3H)-ylidene]pyrrolidine-1-carboxamide;
N-[(2Z)-5-methyl-3-[5-(trifluoromethyl)pyridin-3-yl]-1,3-thiazol-2(3H)-ylidene]pyrrolidine-1-carboxamide;
N-[(2Z)-3-[4-(methylthio)phenyl]-1,3-thiazol-2(3H)-ylidene]pyrrolidine-1-carboxamide;
N-[(2Z)-3-phenyl-1,3-thiazol-2(3H)-ylidene]pyrrolidine-1-carboxamide;
N-[(2Z)-3-(3-chlorophenyl)-1,3-thiazol-2(3H)-ylidene]pyrrolidine-1-carboxamide;
N-[(2Z)-3-[3-(trifluoromethyl)phenyl]-1,3-thiazol-2(3H)-ylidene]pyrrolidine-1-carboxamide;
N-[(2Z)-3-(4-chlorophenyl)-1,3-thiazol-2(3H)-ylidene]pyrrolidine-1-carboxamide;
N-[(2Z)-3-(4-methylphenyl)-1,3-thiazol-2(3H)-ylidene]pyrrolidine-1-carboxamide;
N-[(2Z)-3-(4-chloro-3-methylphenyl)-1,3-thiazol-2(3H)-ylidene]pyrrolidine-1-carboxamide;
N-[(2Z)-3-(3,4-dichlorophenyl)-1,3-thiazol-2(3H)-ylidene]pyrrolidine-1-carboxamide;
N-[(2Z)-3-(3,4-dimethylphenyl)-1,3-thiazol-2(3H)-ylidene]pyrrolidine-1-carboxamide;
N-[(2Z)-3-(4-cyanophenyl)-1,3-thiazol-2(3H)-ylidene]pyrrolidine-1-carboxamide;
N-[(2Z)-3-(3-chloro-4-methylphenyl)-1,3-thiazol-2(3H)-ylidene]pyrrolidine-1-carboxamide;
N-[(2Z)-3-[4-(difluoromethoxy)phenyl]-1,3-thiazol-2(3H)-ylidene]pyrrolidine-1-carboxamide;
N-[(2Z)-3-(4-fluorophenyl)-5-methyl-1,3-thiazol-2(3H)-ylidene]-2,2-dimethylpropanamide;
N-[(2Z)-3-(4-chlorophenyl)-5-methyl-1,3-thiazol-2(3H)-ylidene]-2,2-dimethylpropanamide;
2,2-dimethyl-N-[(2Z)-5-methyl-3-[4-(trifluoromethyl)phenyl]-1,3-thiazol-2(3H)-ylidene]propanamide;
N-[(2Z)-3-[6-(trifluoromethyl)pyridin-3-yl]-1,3-thiazol-2(3H)-ylidene]pyrrolidine-1-carboxamide;
N-[(2Z)-3-[3-fluoro-4-(trifluoromethyl)phenyl]-1,3-thiazol-2(3H)-ylidene]pyrrolidine-1-carboxamide;
N-[(2Z)-3-(6-fluoro-5-methylpyridin-3-yl)-1,3-thiazol-2(3H)-ylidene]pyrrolidine-1-carboxamide;
2,2-dimethyl-N-[(2Z)-5-methyl-3-(5-methylthien-2-yl)-1,3-thiazol-2(3H)-ylidene]propanamide;
N-[(2Z)-3-(6-fluoropyridin-3-yl)-5-methyl-1,3-thiazol-2(3H)-ylidene]-2,2-dimethylpropanamide;
2,2-dimethyl-N-[(2Z)-5-methyl-3-[6-(trifluoromethyl)pyridin-3-yl]-1,3-thiazol-2(3H)-ylidene]propanamide;
N-[(2Z)-3-[3-fluoro-4-(trifluoromethyl)phenyl]-5-methyl-1,3-thiazol-2(3H)-ylidene]-2,2-dimethylpropanamide;
N-[(2Z)-3-(6-fluoro-5-methylpyridin-3-yl)-5-methyl-1,3-thiazol-2(3H)-ylidene]-2,2-dimethylpropanamide;
2,2-dimethyl-N-[(2Z)-5-methyl-3-[6-(1H-pyrazol-1-yl)pyridin-2-yl]-1,3-thiazol-2(3H)-ylidene]propanamide;
N-[(2Z)-3-(2-fluoropyridin-4-yl)-5-methyl-1,3-thiazol-2(3H)-ylidene]-2,2-dimethylpropanamide;
(3R)—N-[(2Z)-3-(3-bromophenyl)-5-methyl-1,3-thiazol-2(3H)-ylidene]-3-fluoropyrrolidine-1-carboxamide;
(3R)—N-[(2Z)-3-(4-cyano-3-methylphenyl)-5-methyl-1,3-thiazol-2(3H)-ylidene]-3-fluoropyrrolidine-1-carboxamide;
(3R)-3-fluoro-N-[(2Z)-5-methyl-3-(3-methylphenyl)-1,3-thiazol-2(3H)-ylidene]pyrrolidine-1-carboxamide;
(3R)—N-[(2Z)-3-(4-bromophenyl)-5-methyl-1,3-thiazol-2(3H)-ylidene]-3-fluoropyrrolidine-1-carboxamide;
(3R)-3-fluoro-N-[(2Z)-5-methyl-3-(4-phenoxyphenyl)-1,3-thiazol-2(3H)-ylidene]pyrrolidine-1-carboxamide;
(3R)-3-fluoro-N-[(2Z)-5-methyl-3-(4-methylphenyl)-1,3-thiazol-2(3H)-ylidene]pyrrolidine-1-carboxamide;
(3R)—N-[(2Z)-3-(4-ethylphenyl)-5-methyl-1,3-thiazol-2(3H)-ylidene]-3-fluoropyrrolidine-1-carboxamide;
(3R)-3-fluoro-N-[(2Z)-3-(3-fluorophenyl)-5-methyl-1,3-thiazol-2(3H)-ylidene]pyrrolidine-1-carboxamide;
(3R)-3-fluoro-N-[(2Z)-3-(4-fluorophenyl)-5-methyl-1,3-thiazol-2(3H)-ylidene]pyrrolidine-1-carboxamide;
(3R)-3-fluoro-N-[(2Z)-5-methyl-3-[3-(trifluoromethyl)phenyl]-1,3-thiazol-2(3H)-ylidene]pyrrolidine-1-carboxamide;
(3R)-3-fluoro-N-[(2Z)-5-methyl-3-[4-(trifluoromethyl)phenyl]-1,3-thiazol-2(3H)-ylidene]pyrrolidine-1-carboxamide;
(3R)—N-[(2Z)-3-(4-chlorophenyl)-5-methyl-1,3-thiazol-2(3H)-ylidene]-3-fluoropyrrolidine-1-carboxamide;
(3R)-3-fluoro-N-[(2Z)-3-(4-iodophenyl)-5-methyl-1,3-thiazol-2(3H)-ylidene]pyrrolidine-1-carboxamide;
(3R)—N-[(2Z)-3-(3-cyanophenyl)-5-methyl-1,3-thiazol-2(3H)-ylidene]-3-fluoropyrrolidine-1-carboxamide;
(3R)-3-fluoro-N-[(2Z)-5-methyl-3-(2-naphthyl)-1,3-thiazol-2(3H)-ylidene]pyrrolidine-1-carboxamide;
(3R)—N-[(2Z)-3-(5-bromothien-2-yl)-5-methyl-1,3-thiazol-2(3H)-ylidene]-3-fluoropyrrolidine-1-carboxamide;
(3R)—N-[(2Z)-3-(3,4-dichlorophenyl)-5-methyl-1,3-thiazol-2(3H)-ylidene]-3-fluoropyrrolidine-1-carboxamide;
(3R)-3-fluoro-N-[(2Z)-3-(3-iodophenyl)-5-methyl-1,3-thiazol-2(3H)-ylidene]pyrrolidine-1-carboxamide;
(3R)—N-[(2Z)-3-(3-chloro-4-methylphenyl)-5-methyl-1,3-thiazol-2(3H)-ylidene]-3-fluoropyrrolidine-1-carboxamide;
(3R)—N-[(2Z)-3-(1-benzothien-5-yl)-5-methyl-1,3-thiazol-2(3H)-ylidene]-3-fluoropyrrolidine-1-carboxamide;
N-[(2Z)-3-(5-bromothien-2-yl)-1,3-thiazol-2(3H)-ylidene]pyrrolidine-1-carboxamide;
N-[(2Z)-3-(2-naphthyl)-1,3-thiazol-2(3H)-ylidene]pyrrolidine-1-carboxamide;
N-[(2Z)-3-(5-chlorothien-2-yl)-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]indoline-1-carboxamide;
N-ethyl-N-methyl-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-propylurea;
N-(1,3-dioxolan-2-ylmethyl)-N-methyl-N′-[(2Z)-5-methyl-3-[4-(trifluoromethyl)phenyl]-1,3-thiazol-2(3H)-ylidene]urea;
N-methyl-N-(3-methylbutyl)-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-prop-2-ynylurea;
N,N-diethyl-N′-[(2Z)-5-methyl-3-[4-(trifluoromethyl)phenyl]-1,3-thiazol-2(3H)-ylidene]urea;
N-ethyl-N′-[(2Z)-5-methyl-3-[4-(trifluoromethyl)phenyl]-1,3-thiazol-2(3H)-ylidene]-N-propylurea;
N-butyl-N-ethyl-N′-[(2Z)-5-methyl-3-[4-(trifluoromethyl)phenyl]-1,3-thiazol-2(3H)-ylidene]urea;
N,N-dibutyl-N′-[(2Z)-5-methyl-3-[4-(trifluoromethyl)phenyl]-1,3-thiazol-2(3H)-ylidene]urea;
2,5-dimethyl-N-[(2Z)-5-methyl-3-[4-(trifluoromethyl)phenyl]-1,3-thiazol-2(3H)-ylidene]pyrrolidine-1-carboxamide;
2-methyl-N-[(2Z)-5-methyl-3-[4-(trifluoromethyl)phenyl]-1,3-thiazol-2(3H)-ylidene]piperidine-1-carboxamide;
N-(2-methoxyethyl)-N-methyl-N′-[(2Z)-5-methyl-3-[4-(trifluoromethyl)phenyl]-1,3-thiazol-2(3H)-ylidene]urea;
N-benzyl-N-ethyl-N′-[(2Z)-5-methyl-3-[4-(trifluoromethyl)phenyl]-1,3-thiazol-2(3H)-ylidene]urea;
N-benzyl-N-isopropyl-N′-[(2Z)-5-methyl-3-[4-(trifluoromethyl)phenyl]-1,3-thiazol-2(3H)-ylidene]urea;
N-benzyl-N-butyl-N′-[(2Z)-5-methyl-3-[4-(trifluoromethyl)phenyl]-1,3-thiazol-2(3H)-ylidene]urea;
4-benzyl-N-[(2Z)-5-methyl-3-[4-(trifluoromethyl)phenyl]-1,3-thiazol-2(3H)-ylidene]piperidine-1-carboxamide;
N-isopropyl-N′-[(2Z)-5-methyl-3-[4-(trifluoromethyl)phenyl]-1,3-thiazol-2(3H)-ylidene]urea;
N-(sec-butyl)-N′-[(2Z)-5-methyl-3-[4-(trifluoromethyl)phenyl]-1,3-thiazol-2(3H)-ylidene]urea;
N-(1-methylbutyl)-N′-[(2Z)-5-methyl-3-[4-(trifluoromethyl)phenyl]-1,3-thiazol-2(3H)-ylidene]urea;
N-(1,1-dimethylpropyl)-N′-[(2Z)-5-methyl-3-[4-(trifluoromethyl)phenyl]-1,3-thiazol-2(3H)-ylidene]urea;
N-(1-ethylpropyl)-N′-[(2Z)-5-methyl-3-[4-(trifluoromethyl)phenyl]-1,3-thiazol-2(3H)-ylidene]urea;
N-(2-methoxy-1-methylethyl)-N′-[(2Z)-5-methyl-3-[4-(trifluoromethyl)phenyl]-1,3-thiazol-2(3H)-ylidene]urea;
N-cyclopropyl-N′-[(2Z)-5-methyl-3-[4-(trifluoromethyl)phenyl]-1,3-thiazol-2(3H)-ylidene]urea;
N-cyclobutyl-N′-[(2Z)-5-methyl-3-[4-(trifluoromethyl)phenyl]-1,3-thiazol-2(3H)-ylidene]urea;
N-cyclopentyl-N′-[(2Z)-5-methyl-3-[4-(trifluoromethyl)phenyl]-1,3-thiazol-2(3H)-ylidene]urea;
(3R)—N-[(2Z)-3-(3,5-dichlorophenyl)-5-(hydroxymethyl)-1,3-thiazol-2(3H)-ylidene]-3-fluoropyrrolidine-1-carboxamide;
(3R)—N-[(2Z)-3-(3-chlorophenyl)-5-(hydroxymethyl)-1,3-thiazol-2(3H)-ylidene]-3-fluoropyrrolidine-1-carboxamide;
(3R)—N-[(2Z)-3-(3,4-dimethylphenyl)-5-(hydroxymethyl)-1,3-thiazol-2(3H)-ylidene]-3-fluoropyrrolidine-1-carboxamide;
(3R)—N-[(2Z)-3-(3,5-dimethylphenyl)-5-(hydroxymethyl)-1,3-thiazol-2(3H)-ylidene]-3-fluoropyrrolidine-1-carboxamide;
(3R)—N-[(2Z)-3-(4-chloro-3-methylphenyl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-[4-(trifluoromethoxy)phenyl]-1,3-thiazol-2(3H)-ylidene]pyrrolidine-1-carboxamide;
(3R)—N-[(2Z)-3-(3,4-dichlorophenyl)-5-(hydroxymethyl)-1,3-thiazol-2(3H)-ylidene]-3-fluoropyrrolidine-1-carboxamide;
(3R)-3-fluoro-N-[(2Z)-3-[3-fluoro-4-(trifluoromethyl)phenyl]-5-(hydroxymethyl)-1,3-thiazol-2(3H)-ylidene]pyrrolidine-1-carboxamide;
N-[(2Z)-3-(4-chlorophenyl)-4,5-dimethyl-1,3-thiazol-2(3H)-ylidene]pyrrolidine-1-carboxamide;
N-[(2Z)-4,5-dimethyl-3-[4-(trifluoromethyl)phenyl]-1,3-thiazol-2(3H)-ylidene]pyrrolidine-1-carboxamide;
N-[(2Z)-3-(6-chloropyridin-3-yl)-4,5-dimethyl-1,3-thiazol-2(3H)-ylidene]pyrrolidine-1-carboxamide;
N-[(2Z)-3-(6-chloropyridin-3-yl)-5-methyl-1,3-thiazol-2(3H)-ylidene]pyrrolidine-1-carboxamide;
4-acetyl-N-[(2Z)-5-methyl-3-[4-(trifluoromethyl)phenyl]-1,3-thiazol-2(3H)-ylidene]piperazine-1-carboxamide;
{(2Z)-2-(acetylimino)-3-[4-(trifluoromethyl)phenyl]-2,3-dihydro-1,3-thiazol-5-yl}methyl acetate;
N-[(2Z)-3-(3-methyl-1-benzothien-5-yl)-1,3-thiazol-2(3H)-ylidene]pyrrolidine-1-carboxamide;
N-methyl-N′-[(2Z)-5-methyl-3-[4-(trifluoromethyl)phenyl]-1,3-thiazol-2(3H)-ylidene]-N-pentylurea;
N-[(2′Z)-5-chloro-5′-methyl-2,3′-bi-1,3-thiazol-2′-ylidene]pyrrolidine-1-carboxamide;
N-[(2Z)-3-(4-fluorophenyl)-1,3-thiazol-2(3H)-ylidene]pyrrolidine-1-carboxamide;
N-[(2Z)-3-(5-methylthien-2-yl)-1,3-thiazol-2(3H)-ylidene]pyrrolidine-1-carboxamide;
(3R)—N-[(2Z)-3-(2-chloro-1-benzothien-5-yl)-5-methyl-1,3-thiazol-2(3H)-ylidene]-3-fluoropyrrolidine-1-carboxamide;
(3R)-3-fluoro-N-[(2Z)-5-methyl-3-[1,2,4]triazolo[1,5-a]pyridin-6-yl-1,3-thiazol-2(3H)-ylidene]pyrrolidine-1-carboxamide;
N-[(2Z)-3-[2,4-bis(trifluoromethyl)phenyl]-5-methyl-1,3-thiazol-2(3H)-ylidene]-2,2-dimethylpropanamide;
N-[(2Z)-3-[4-(difluoromethoxy)phenyl]-5-methyl-1,3-thiazol-2(3H)-ylidene]-2,2-dimethylpropanamide;
N-[(2Z)-3-(5-pyridin-2-ylthien-2-yl)-1,3-thiazol-2(3H)-ylidene]pyrrolidine-1-carboxamide;
N-[(2Z)-3-(1-benzofuran-2-yl)-1,3-thiazol-2(3H)-ylidene]pyrrolidine-1-carboxamide;
N-[(2Z)-3-(5-methyl-1-benzothien-2-yl)-1,3-thiazol-2(3H)-ylidene]pyrrolidine-1-carboxamide;
N-[(2Z)-3-thien-2-yl-1,3-thiazol-2(3H)-ylidene]pyrrolidine-1-carboxamide;
N-[(2Z)-3-(2,2′-bithien-5-yl)-1,3-thiazol-2(3H)-ylidene]pyrrolidine-1-carboxamide;
N-[(2Z)-3-(5-cyanothien-2-yl)-1,3-thiazol-2(3H)-ylidene]pyrrolidine-1-carboxamide;
N-[(2Z)-3-(4-chlorophenyl)-1,3-thiazolidin-2-ylidene]-1H-imidazole-1-carboxamide;
(3R)—N-[(2Z)-3-[5-(aminocarbonyl)thien-2-yl]-5-methyl-1,3-thiazol-2(3H)-ylidene]-3-fluoropyrrolidine-1-carboxamide;
N′-[(2Z)-3-(5-chlorothien-2-yl)-5-methyl-1,3-thiazol-2(3H)-ylidene]-N-(3-fluorobenzyl)-N-methylurea;
(3R)—N-[(2Z)-3-(4-cyanophenyl)-5-(hydroxymethyl)-1,3-thiazol-2(3H)-ylidene]-3-fluoropyrrolidine-1-carboxamide;
(3R)-3-fluoro-N-[(2Z)-5-(hydroxymethyl)-3-(5,6,8-trifluoro-2-naphthyl)-1,3-thiazol-2(3H)-ylidene]pyrrolidine-1-carboxamide;
N-[(2Z)-3-(4-methoxyphenyl)-5-methylene-1,3-thiazolidin-2-ylidene]acetamide;
N-[(2Z)-3-(4-methoxyphenyl)-5-methyl-1,3-thiazol-2(3H)-ylidene]acetamide;
N-{(2Z)-3-[4-(difluoromethoxy)phenyl]-5-methylene-1,3-thiazolidin-2-ylidene}acetamide;
N-[(2Z)-3-[4-(difluoromethoxy)phenyl]-5-methyl-1,3-thiazol-2(3H)-ylidene]acetamide;
N′-[(2Z)-3-[4-(difluoromethoxy)phenyl]-5-methyl-1,3-thiazol-2(3H)-ylidene]-N,N-diisopropylurea;
N′-[(2Z)-3-[4-(difluoromethoxy)phenyl]-5-methyl-1,3-thiazol-2(3H)-ylidene]-N-methyl-N-phenylurea;
N′-[(2Z)-3-[4-(difluoromethoxy)phenyl]-5-methyl-1,3-thiazol-2(3H)-ylidene]-N,N-dimethylurea;
N-{(2Z)-5-methylene-3-[4-(trifluoromethoxy)phenyl]-1,3-thiazolidin-2-ylidene}acetamide;
N-[(2Z)-5-methyl-3-[4-(trifluoromethoxy)phenyl]-1,3-thiazol-2(3H)-ylidene]acetamide;
N-[(2Z)-5-methyl-3-[4-(trifluoromethoxy)phenyl]-1,3-thiazol-2(3H)-ylidene]piperidine-1-carboxamide;
N,N-diisopropyl-N′-[(2Z)-5-methyl-3-[4-(trifluoromethoxy)phenyl]-1,3-thiazol-2(3H)-ylidene]urea;
N-methyl-N′-[(2Z)-5-methyl-3-[4-(trifluoromethoxy)phenyl]-1,3-thiazol-2(3H)-ylidene]-N-phenylurea;
N-[(2Z)-3-(4-chlorophenyl)-5-methylene-1,3-thiazolidin-2-ylidene]acetamide;
N-[(2Z)-3-(4-chlorophenyl)-5-methyl-1,3-thiazol-2(3H)-ylidene]acetamide;
N′-[(2Z)-3-(4-chlorophenyl)-5-methyl-1,3-thiazol-2(3H)-ylidene]-N,N-diisopropylurea;
N-[(2Z)-3-(6,7-dicyano-2-naphthyl)-5-methyl-1,3-thiazol-2(3H)-ylidene]-2,2-dimethylpropanamide;
N-[(2Z)-3-(4-benzylphenyl)-5-methyl-1,3-thiazol-2(3H)-ylidene]-2,2-dimethylpropanamide;
N′-[(2Z)-3-(4-fluorophenyl)-5-methyl-1,3-thiazol-2(3H)-ylidene]-N,N-diisopropylurea;
N-[(2Z)-3-[4-(cyanomethyl)phenyl]-5-methyl-1,3-thiazol-2(3H)-ylidene]pyrrolidine-1-carboxamide;
N-[(2Z)-3-[4-(cyanomethyl)phenyl]-5-methyl-1,3-thiazol-2(3H)-ylidene]piperidine-1-carboxamide;
N′-[(2Z)-3-[4-(cyanomethyl)phenyl]-5-methyl-1,3-thiazol-2(3H)-ylidene]-N,N-diethylurea;
N′-[(2Z)-3-[4-(cyanomethyl)phenyl]-5-methyl-1,3-thiazol-2(3H)-ylidene]-N,N-dimethylurea;
N-[(2Z)-5-methyl-3-[4-(trifluoromethyl)phenyl]-1,3-thiazol-2(3H)-ylidene]piperidine-1-carboxamide;
N-[(2Z)-3-[4-(cyanomethyl)phenyl]-5-methyl-1,3-thiazol-2(3H)-ylidene]-2,2-dimethylpropanamide;
N-[(2Z)-3-(2,6-dimethylpyrimidin-4-yl)-5-methyl-1,3-thiazol-2(3H)-ylidene]-2,2-dimethylpropanamide;
N-[(2Z)-5-methyl-3-[4-(trifluoromethyl)phenyl]-1,3-thiazol-2(3H)-ylidene]-1H-imidazole-1-carboxamide;
tert-butyl (2R,3S)-2-methyl-1-({[(2Z)-5-methyl-3-[4-(trifluoromethyl)phenyl]-1,3-thiazol-2(3H)-ylidene]amino}carbonyl)pyrrolidin-3-ylcarbamate;
2-isopropyl-N-[(2Z)-5-methyl-3-[4-(trifluoromethyl)phenyl]-1,3-thiazol-2(3H)-ylidene]pyrrolidine-1-carboxamide;
2-isobutyl-N-[(2Z)-5-methyl-3-[4-(trifluoromethyl)phenyl]-1,3-thiazol-2(3H)-ylidene]pyrrolidine-1-carboxamide;
(3R)-3-(dimethylamino)-N-[(2Z)-5-methyl-3-[4-(trifluoromethyl)phenyl]-1,3-thiazol-2(3H)-ylidene]pyrrolidine-1-carboxamide;
tert-butyl (3S)-1-({[(2Z)-5-methyl-3-[4-(trifluoromethyl)phenyl]-1,3-thiazol-2(3H)-ylidene]amino}carbonyl)pyrrolidin-3-ylcarbamate;
tert-butyl 5-({[(2Z)-5-methyl-3-[4-(trifluoromethyl)phenyl]-1,3-thiazol-2(3H)-ylidene]amino}carbonyl)hexahydropyrrolo[3,4-c]pyrrole-2(1H)-carboxylate;
tert-butyl (1S,5R)-3-({[(2Z)-5-methyl-3-[4-(trifluoromethyl)phenyl]-1,3-thiazol-2(3H)-ylidene]amino}carbonyl)-3,6-diazabicyclo[3.2.0]heptane-6-carboxylate;
tert-butyl (3R)-3-methyl-1-({[(2Z)-5-methyl-3-[4-(trifluoromethyl)phenyl]-1,3-thiazol-2(3H)-ylidene]amino}carbonyl)pyrrolidin-3-ylcarbamate;
tert-butyl 6-({[(2Z)-5-methyl-3-[4-(trifluoromethyl)phenyl]-1,3-thiazol-2(3H)-ylidene]amino}carbonyl)octahydro-1H-pyrrolo[3,4-b]pyridine-1-carboxylate;
tert-butyl (1S,4S)-5-({[(2Z)-5-methyl-3-[4-(trifluoromethyl)phenyl]-1,3-thiazol-2(3H)-ylidene]amino}carbonyl)-2,5-diazabicyclo[2.2.1]heptane-2-carboxylate;
(3S)-3-amino-N-[(2Z)-5-methyl-3-[4-(trifluoromethyl)phenyl]-1,3-thiazol-2(3H)-ylidene]pyrrolidine-1-carboxamide;
(2R,3S)-3-amino-2-methyl-N-[(2Z)-5-methyl-3-[4-(trifluoromethyl)phenyl]-1,3-thiazol-2(3H)-ylidene]pyrrolidine-1-carboxamide;
N-[(2Z)-5-methyl-3-[4-(trifluoromethyl)phenyl]-1,3-thiazol-2(3H)-ylidene]hexahydropyrrolo[3,4-c]pyrrole-2(1H)-carboxamide;
(3R)-3-amino-3-methyl-N-[(2Z)-5-methyl-3-[4-(trifluoromethyl)phenyl]-1,3-thiazol-2(3H)-ylidene]pyrrolidine-1-carboxamide;
(1R,5R)—N-[(2Z)-5-methyl-3-[4-(trifluoromethyl)phenyl]-1,3-thiazol-2(3H)-ylidene]-3,6-diazabicyclo[3.2.0]heptane-3-carboxamide;
(4aS,7aS)—N-[(2Z)-5-methyl-3-[4-(trifluoromethyl)phenyl]-1,3-thiazol-2(3H)-ylidene]octahydro-6H-pyrrolo[3,4-b]pyridine-6-carboxamide;
(1S,4S)—N-[(2Z)-5-methyl-3-[4-(trifluoromethyl)phenyl]-1,3-thiazol-2(3H)-ylidene]-2,5-diazabicyclo[2.2.1]heptane-2-carboxamide;
N′-[(2Z)-3-(4-chlorophenyl)-4,5-dimethyl-1,3-thiazol-2(3H)-ylidene]-N,N-dimethylurea;
N′-[(2Z)-4,5-dimethyl-3-[4-(trifluoromethyl)phenyl]-1,3-thiazol-2(3H)-ylidene]-N,N-dimethylurea;
N′-[(2Z)-3-(3-ethoxyphenyl)-4,5-dimethyl-1,3-thiazol-2(3H)-ylidene]-N,N-dimethylurea;
N-[(2Z)-3-(3-ethoxyphenyl)-4,5-dimethyl-1,3-thiazol-2(3H)-ylidene]pyrrolidine-1-carboxamide;
N′-[(2Z)-3-(4-chlorophenyl)-4-phenyl-1,3-thiazol-2(3H)-ylidene]-N,N-dimethylurea;
N-isopropyl-N-methyl-N′-[(2Z)-5-methyl-3-[4-(trifluoromethyl)phenyl]-1,3-thiazol-2(3H)-ylidene]urea;
N-butyl-N-methyl-N′-[(2Z)-5-methyl-3-[4-(trifluoromethyl)phenyl]-1,3-thiazol-2(3H)-ylidene]urea;
N-isobutyl-N-methyl-N′-[(2Z)-5-methyl-3-[4-(trifluoromethyl)phenyl]-1,3-thiazol-2(3H)-ylidene]urea;
N-cyclohexyl-N-methyl-N′-[(2Z)-5-methyl-3-[4-(trifluoromethyl)phenyl]-1,3-thiazol-2(3H)-ylidene]urea;
N-ethyl-N-isopropyl-N′-[(2Z)-5-methyl-3-[4-(trifluoromethyl)phenyl]-1,3-thiazol-2(3H)-ylidene]urea;
N-cyclohexyl-N-ethyl-N′-[(2Z)-5-methyl-3-[4-(trifluoromethyl)phenyl]-1,3-thiazol-2(3H)-ylidene]urea;
N′-[(2Z)-5-methyl-3-[4-(trifluoromethyl)phenyl]-1,3-thiazol-2(3H)-ylidene]-N,N-dipropylurea;
N-isopropyl-N′-[(2Z)-5-methyl-3-[4-(trifluoromethyl)phenyl]-1,3-thiazol-2(3H)-ylidene]-N-propylurea;
N-butyl-N′-[(2Z)-5-methyl-3-[4-(trifluoromethyl)phenyl]-1,3-thiazol-2(3H)-ylidene]-N-propylurea;
N-(sec-butyl)-N′-[(2Z)-5-methyl-3-[4-(trifluoromethyl)phenyl]-1,3-thiazol-2(3H)-ylidene]-N-propylurea;
N-(cyclopropylmethyl)-N′-[(2Z)-5-methyl-3-[4-(trifluoromethyl)phenyl]-1,3-thiazol-2(3H)-ylidene]-N-propylurea;
N-(cyanomethyl)-N-methyl-N′-[(2Z)-5-methyl-3-[4-(trifluoromethyl)phenyl]-1,3-thiazol-2(3H)-ylidene]urea;
N-(2-cyanoethyl)-N-cyclopropyl-N′-[(2Z)-5-methyl-3-[4-(trifluoromethyl)phenyl]-1,3-thiazol-2(3H)-ylidene]urea;
N,N-diisobutyl-N′-[(2Z)-5-methyl-3-[4-(trifluoromethyl)phenyl]-1,3-thiazol-2(3H)-ylidene]urea;
3-methyl-N-[(2Z)-5-methyl-3-[4-(trifluoromethyl)phenyl]-1,3-thiazol-2(3H)-ylidene]piperidine-1-carboxamide;
4-methyl-N-[(2Z)-5-methyl-3-[4-(trifluoromethyl)phenyl]-1,3-thiazol-2(3H)-ylidene]piperidine-1-carboxamide;
2-ethyl-N-[(2Z)-5-methyl-3-[4-(trifluoromethyl)phenyl]-1,3-thiazol-2(3H)-ylidene]piperidine-1-carboxamide;
N-[(2Z)-5-methyl-3-[4-(trifluoromethyl)phenyl]-1,3-thiazol-2(3H)-ylidene]-4-propylpiperidine-1-carboxamide;
N-[(2Z)-5-methyl-3-[4-(trifluoromethyl)phenyl]-1,3-thiazol-2(3H)-ylidene]azepane-1-carboxamide;
N,N-bis(2-ethoxyethyl)-N′-[(2Z)-5-methyl-3-[4-(trifluoromethyl)phenyl]-1,3-thiazol-2(3H)-ylidene]urea;
N-ethyl-N-(2-methoxyethyl)-N′-[(2Z)-5-methyl-3-[4-(trifluoromethyl)phenyl]-1,3-thiazol-2(3H)-ylidene]urea;
N-(2-methoxyethyl)-N′-[(2Z)-5-methyl-3-[4-(trifluoromethyl)phenyl]-1,3-thiazol-2(3H)-ylidene]-N-propylurea;
N-isopropyl-N-(2-methoxyethyl)-N′-[(2Z)-5-methyl-3-[4-(trifluoromethyl)phenyl]-1,3-thiazol-2(3H)-ylidene]urea;
N,N-bis(2-methoxyethyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]morpholine-4-carboxamide;
N-[(2Z)-5-methyl-3-[4-(trifluoromethyl)phenyl]-1,3-thiazol-2(3H)-ylidene]thiomorpholine-4-carboxamide;
N-(2-cyanoethyl)-N-methyl-N′-[(2Z)-5-methyl-3-[4-(trifluoromethyl)phenyl]-1,3-thiazol-2(3H)-ylidene]urea;
N-benzyl-N′-[(2Z)-5-methyl-3-[4-(trifluoromethyl)phenyl]-1,3-thiazol-2(3H)-ylidene]urea;
N-(3-chlorobenzyl)-N′-[(2Z)-5-methyl-3-[4-(trifluoromethyl)phenyl]-1,3-thiazol-2(3H)-ylidene]urea;
N-(3,5-dichlorobenzyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-(2-phenylethyl)urea;
N-[(2Z)-5-methyl-3-[4-(trifluoromethyl)phenyl]-1,3-thiazol-2(3H)-ylidene]-N′-(2-pyridin-2-ylethyl)urea;
N-[(2Z)-5-methyl-3-[4-(trifluoromethyl)phenyl]-1,3-thiazol-2(3H)-ylidene]-N′-(2-pyridin-3-ylethyl)urea;
N-[(2Z)-5-methyl-3-[4-(trifluoromethyl)phenyl]-1,3-thiazol-2(3H)-ylidene]-N′-(2-pyridin-4-ylethyl)urea;
N-[(2Z)-5-methyl-3-[4-(trifluoromethyl)phenyl]-1,3-thiazol-2(3H)-ylidene]-N′-(pyridin-3-ylmethyl)urea;
N-[2-(dimethylamino)ethyl]-N′-[(2Z)-5-methyl-3-[4-(trifluoromethyl)phenyl]-1,3-thiazol-2(3H)-ylidene]urea;
N-[3-(dimethylamino)propyl]-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-(2-pyrrolidin-1-ylethyl)urea;
N-[(2Z)-5-methyl-3-[4-(trifluoromethyl)phenyl]-1,3-thiazol-2(3H)-ylidene]-N′-(3-pyrrolidin-1-ylpropyl)urea;
N-[2-(4-methylpiperazin-1-yl)ethyl]-N′-[(2Z)-5-methyl-3-[4-(trifluoromethyl)phenyl]-1,3-thiazol-2(3H)-ylidene]urea;
N-[2-(dimethylamino)ethyl]-N-methyl-N′-[(2Z)-5-methyl-3-[4-(trifluoromethyl)phenyl]-1,3-thiazol-2(3H)-ylidene]urea;
N-[3-(dimethylamino)propyl]-N-methyl-N′-[(2Z)-5-methyl-3-[4-(trifluoromethyl)phenyl]-1,3-thiazol-2(3H)-ylidene]urea;
N-[2-(dimethylamino)ethyl]-N-ethyl-N′-[(2Z)-5-methyl-3-[4-(trifluoromethyl)phenyl]-1,3-thiazol-2(3H)-ylidene]urea;
N-[2-(diethylamino)ethyl]-N-methyl-N′-[(2Z)-5-methyl-3-[4-(trifluoromethyl)phenyl]-1,3-thiazol-2(3H)-ylidene]urea;
N,N-dibenzyl-N′-[(2Z)-5-methyl-3-[4-(trifluoromethyl)phenyl]-1,3-thiazol-2(3H)-ylidene]urea;
N-methyl-N′-[(2Z)-5-methyl-3-[4-(trifluoromethyl)phenyl]-1,3-thiazol-2(3H)-ylidene]-N-(2-phenylethyl)urea;
N-methyl-N′-[(2Z)-5-methyl-3-[4-(trifluoromethyl)phenyl]-1,3-thiazol-2(3H)-ylidene]-N-(2-pyridin-2-ylethyl)urea;
4-hydroxy-N-[(2Z)-5-methyl-3-[4-(trifluoromethyl)phenyl]-1,3-thiazol-2(3H)-ylidene]piperidine-1-carboxamide;
2,6-dimethyl-N-[(2Z)-5-methyl-3-[4-(trifluoromethyl)phenyl]-1,3-thiazol-2(3H)-ylidene]morpholine-4-carboxamide;
4-methyl-N-[(2Z)-5-methyl-3-[4-(trifluoromethyl)phenyl]-1,3-thiazol-2(3H)-ylidene]piperazine-1-carboxamide;
4-ethyl-N-[(2Z)-5-methyl-3-[4-(trifluoromethyl)phenyl]-1,3-thiazol-2(3H)-ylidene]piperazine-1-carboxamide;
N-[(2Z)-5-methyl-3-[4-(trifluoromethyl)phenyl]-1,3-thiazol-2(3H)-ylidene]-4-phenylpiperazine-1-carboxamide;
N-[(2Z)-5-methyl-3-[4-(trifluoromethyl)phenyl]-1,3-thiazol-2(3H)-ylidene]-4-pyridin-2-ylpiperazine-1-carboxamide;
4-methyl-N-[(2Z)-5-methyl-3-[4-(trifluoromethyl)phenyl]-1,3-thiazol-2(3H)-ylidene]-1,4-diazepane-1-carboxamide;
N-[(2Z)-5-methyl-3-[4-(trifluoromethyl)phenyl]-1,3-thiazol-2(3H)-ylidene]-4-pyrimidin-2-ylpiperazine-1-carboxamide;
N-isobutyl-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-pentylurea;
N-(3-methylbutyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-neopentylurea;
N-(2-methoxyethyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-[(2S)-tetrahydrofuran-2-ylmethyl]urea;
N-[(2Z)-5-methyl-3-[4-(trifluoromethyl)phenyl]-1,3-thiazol-2(3H)-ylidene]-N′-[(2R)-tetrahydrofuran-2-ylmethyl]urea;
N-[(2Z)-5-methyl-3-[4-(trifluoromethyl)phenyl]-1,3-thiazol-2(3H)-ylidene]-N′-(tetrahydrofuran-3-ylmethyl)urea;
N-[(2Z)-5-methyl-3-[4-(trifluoromethyl)phenyl]-1,3-thiazol-2(3H)-ylidene]-N′-prop-2-ynylurea;
N-(cyclopropylmethyl)-N′-[(2Z)-5-methyl-3-[4-(trifluoromethyl)phenyl]-1,3-thiazol-2(3H)-ylidene]urea;
N-(cyclopentylmethyl)-N′-[(2Z)-5-methyl-3-[4-(trifluoromethyl)phenyl]-1,3-thiazol-2(3H)-ylidene]urea;
N-cyclohexyl-N′-[(2Z)-5-methyl-3-[4-(trifluoromethyl)phenyl]-1,3-thiazol-2(3H)-ylidene]urea;
N-cycloheptyl-N′-[(2Z)-5-methyl-3-[4-(trifluoromethyl)phenyl]-1,3-thiazol-2(3H)-ylidene]urea;
N-1-adamantyl-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-phenylurea;
N-(2-methylphenyl)-N′-[(2Z)-5-methyl-3-[4-(trifluoromethyl)phenyl]-1,3-thiazol-2(3H)-ylidene]urea;
N-(3-methylphenyl)-N′-[(2Z)-5-methyl-3-[4-(trifluoromethyl)phenyl]-1,3-thiazol-2(3H)-ylidene]urea;
N-(4-methylphenyl)-N′-[(2Z)-5-methyl-3-[4-(trifluoromethyl)phenyl]-1,3-thiazol-2(3H)-ylidene]urea;
N-(3-methoxyphenyl)-N′-[(2Z)-5-methyl-3-[4-(trifluoromethyl)phenyl]-1,3-thiazol-2(3H)-ylidene]urea;
N-(3-fluorophenyl)-N′-[(2Z)-5-methyl-3-[4-(trifluoromethyl)phenyl]-1,3-thiazol-2(3H)-ylidene]urea;
N-(3-chlorophenyl)-N′-[(2Z)-5-methyl-3-[4-(trifluoromethyl)phenyl]-1,3-thiazol-2(3H)-ylidene]urea;
N-(3-bromophenyl)-N′-[(2Z)-5-methyl-3-[4-(trifluoromethyl)phenyl]-1,3-thiazol-2(3H)-ylidene]urea;
N-[(2Z)-5-methyl-3-[4-(trifluoromethyl)phenyl]-1,3-thiazol-2(3H)-ylidene]-N′-[3-(trifluoromethyl)phenyl]urea;
N-(3,5-dichlorophenyl)-N′-[(2Z)-5-methyl-3-[4-(trifluoromethyl)phenyl]-1,3-thiazol-2(3H)-ylidene]urea;
N-methyl-N-(4-methylphenyl)-N′-[(2Z)-5-methyl-3-[4-(trifluoromethyl)phenyl]-1,3-thiazol-2(3H)-ylidene]urea;
N-{(2Z)-5-methyl-3-[4-(trifluoromethyl)phenyl]-1,3-thiazolidin-2-ylidene}acetamide;
N-[(2Z)-4,5-dimethyl-3-(6-morpholin-4-ylpyridin-3-yl)-1,3-thiazol-2(3H)-ylidene]pyrrolidine-1-carboxamide;
N-{(2Z)-5-methylene-3-[4-(trifluoromethyl)phenyl]-1,3-thiazolidin-2-ylidene}pyrrolidine-1-carboxamide;
N-{(2Z)-4-hydroxy-5-methylene-3-[4-(trifluoromethyl)phenyl]-1,3-thiazolidin-2-ylidene}pyrrolidine-1-carboxamide;
N-{(2Z)-5-methylene-4-(1H-pyrazol-1-yl)-3-[4-(trifluoromethyl)phenyl]-1,3-thiazolidin-2-ylidene}acetamide;
N-[(2Z)-5-(1H-pyrazol-1-ylmethyl)-3-[4-(trifluoromethyl)phenyl]-1,3-thiazol-2(3H)-ylidene]acetamide;
N-{(2Z,5Z)-5-(iodomethylene)-3-[4-(trifluoromethyl)phenyl]-1,3-thiazolidin-2-ylidene}acetamide;
N-[(2Z)-5-(cyanomethyl)-3-[4-(trifluoromethyl)phenyl]-1,3-thiazol-2(3H)-ylidene]acetamide;
N-[(2Z)-5-[(ethylamino)methyl]-3-[4-(trifluoromethyl)phenyl]-1,3-thiazol-2(3H)-ylidene]acetamide;
N-[(2Z)-5-[(propylamino)methyl]-3-[4-(trifluoromethyl)phenyl]-1,3-thiazol-2(3H)-ylidene]acetamide;
N-[(2Z)-5-[(butylamino)methyl]-3-[4-(trifluoromethyl)phenyl]-1,3-thiazol-2(3H)-ylidene]acetamide;
N-[(2Z)-5-[(tert-butylamino)methyl]-3-[4-(trifluoromethyl)phenyl]-1,3-thiazol-2(3H)-ylidene]acetamide;
N-[(2Z)-5-{[(cyanomethyl)amino]methyl}-3-[4-(trifluoromethyl)phenyl]-1,3-thiazol-2(3H)-ylidene]acetamide;
N-[(2Z)-5-[(dimethylamino)methyl]-3-[4-(trifluoromethyl)phenyl]-1,3-thiazol-2(3H)-ylidene]acetamide;
N-[(2Z)-5-{[butyl(methyl)amino]methyl}-3-[4-(trifluoromethyl)phenyl]-1,3-thiazol-2(3H)-ylidene]acetamide;
N-[(2Z)-5-{[(1,3-dioxolan-2-ylmethyl)(methyl)amino]methyl}-3-[4-(trifluoromethyl)phenyl]-1,3-thiazol-2(3H)-ylidene]acetamide;
N-[(2Z)-5-[(diethylamino)methyl]-3-[4-(trifluoromethyl)phenyl]-1,3-thiazol-2(3H)-ylidene]acetamide;
N-[(2Z)-5-(azetidin-1-ylmethyl)-3-[4-(trifluoromethyl)phenyl]-1,3-thiazol-2(3H)-ylidene]acetamide;
N-[(2Z)-5-{[bis(2-methoxyethyl)amino]methyl}-3-[4-(trifluoromethyl)phenyl]-1,3-thiazol-2(3H)-ylidene]acetamide;
N-[(2Z)-5-{[(1-phenylethyl)amino]methyl}-3-[4-(trifluoromethyl)phenyl]-1,3-thiazol-2(3H)-ylidene]acetamide;
N-[(2Z)-5-methyl-3-(2-methylphenyl)-1,3-thiazol-2(3H)-ylidene]pyrrolidine-1-carboxamide;
N-[(2Z)-3-(2-aminophenyl)-5-methyl-1,3-thiazol-2(3H)-ylidene]pyrrolidine-1-carboxamide;
N-[(2Z)-3-(2-hydroxyphenyl)-5-methyl-1,3-thiazol-2(3H)-ylidene]pyrrolidine-1-carboxamide;
N-[(2Z)-3-(4-hydroxyphenyl)-5-methyl-1,3-thiazol-2(3H)-ylidene]pyrrolidine-1-carboxamide;
N-[(2Z)-3-(2-methoxyphenyl)-5-methyl-1,3-thiazol-2(3H)-ylidene]pyrrolidine-1-carboxamide;
N-[(2Z)-3-(2-fluorophenyl)-5-methyl-1,3-thiazol-2(3H)-ylidene]pyrrolidine-1-carboxamide;
N-[(2Z)-3-(2-chlorophenyl)-5-methyl-1,3-thiazol-2(3H)-ylidene]pyrrolidine-1-carboxamide;
N-[(2Z)-3-[3-(dimethylamino)phenyl]-5-methyl-1,3-thiazol-2(3H)-ylidene]pyrrolidine-1-carboxamide;
N-[(2Z)-3-(2,3-dimethylphenyl)-5-methyl-1,3-thiazol-2(3H)-ylidene]pyrrolidine-1-carboxamide;
N-[(2Z)-3-(2,4-dimethylphenyl)-5-methyl-1,3-thiazol-2(3H)-ylidene]pyrrolidine-1-carboxamide;
N-[(2Z)-3-(2,5-dimethylphenyl)-5-methyl-1,3-thiazol-2(3H)-ylidene]pyrrolidine-1-carboxamide;
N-[(2Z)-3-(2,4-dimethoxyphenyl)-5-methyl-1,3-thiazol-2(3H)-ylidene]pyrrolidine-1-carboxamide;
N-[(2Z)-3-(2,5-dimethoxyphenyl)-5-methyl-1,3-thiazol-2(3H)-ylidene]pyrrolidine-1-carboxamide;
N-[(2Z)-3-(3,4-dimethoxyphenyl)-5-methyl-1,3-thiazol-2(3H)-ylidene]pyrrolidine-1-carboxamide;
N-[(2Z)-5-methyl-3-(3,4,5-trimethoxyphenyl)-1,3-thiazol-2(3H)-ylidene]pyrrolidine-1-carboxamide;
N-[(2Z)-3-(2,3-dichlorophenyl)-5-methyl-1,3-thiazol-2(3H)-ylidene]pyrrolidine-1-carboxamide;
N-[(2Z)-3-(2,4-dichlorophenyl)-5-methyl-1,3-thiazol-2(3H)-ylidene]pyrrolidine-1-carboxamide;
N-[(2Z)-3-(2,5-dichlorophenyl)-5-methyl-1,3-thiazol-2(3H)-ylidene]pyrrolidine-1-carboxamide;
N-[(2Z)-3-(4-aminophenyl)-5-methyl-1,3-thiazol-2(3H)-ylidene]pyrrolidine-1-carboxamide;
N-[(2Z)-5-methyl-3-(3-propionylphenyl)-1,3-thiazol-2(3H)-ylidene]pyrrolidine-1-carboxamide;
N-[(2Z)-3-(4-tert-butylphenyl)-5-methyl-1,3-thiazol-2(3H)-ylidene]pyrrolidine-1-carboxamide;
N-[(2Z)-3-(4-ethylphenyl)-5-methyl-1,3-thiazol-2(3H)-ylidene]pyrrolidine-1-carboxamide;
N-[(2Z)-5-methyl-3-(4-propylphenyl)-1,3-thiazol-2(3H)-ylidene]pyrrolidine-1-carboxamide;
N-[(2Z)-5-methyl-3-pyridin-3-yl-1,3-thiazol-2(3H)-ylidene]pyrrolidine-1-carboxamide;
N-[(2Z)-3-(4-butylphenyl)-5-methyl-1,3-thiazol-2(3H)-ylidene]pyrrolidine-1-carboxamide;
N-[(2Z)-5-methyl-3-[3-(methylthio)phenyl]-1,3-thiazol-2(3H)-ylidene]pyrrolidine-1-carboxamide;
N-[(2Z)-5-methyl-3-[2,3,5,6-tetrafluoro-4-(trifluoromethyl)phenyl]-1,3-thiazol-2(3H)-ylidene]pyrrolidine-1-carboxamide;
N-[(2Z)-3-(3-isopropoxyphenyl)-5-methyl-1,3-thiazol-2(3H)-ylidene]pyrrolidine-1-carboxamide;
N-[(2Z)-3-[2,4-bis(trifluoromethyl)phenyl]-5-methyl-1,3-thiazol-2(3H)-ylidene]pyrrolidine-1-carboxamide;
N-[(2Z)-3-(4-chloro-3-methoxyphenyl)-5-methyl-1,3-thiazol-2(3H)-ylidene]pyrrolidine-1-carboxamide;
N-[(2Z)-5-methyl-3-(1-oxo-2,3-dihydro-1H-inden-5-yl)-1,3-thiazol-2(3H)-ylidene]pyrrolidine-1-carboxamide;
N-[(2Z)-3-(4-methoxy-3-methylphenyl)-5-methyl-1,3-thiazol-2(3H)-ylidene]pyrrolidine-1-carboxamide;
N-[(2Z)-3-(4-cyclohexylphenyl)-5-methyl-1,3-thiazol-2(3H)-ylidene]pyrrolidine-1-carboxamide;
N-[(2Z)-5-methyl-3-(5-phenylthien-2-yl)-1,3-thiazol-2(3H)-ylidene]pyrrolidine-1-carboxamide;
N-[(2Z)-5-methyl-3-(1-naphthyl)-1,3-thiazol-2(3H)-ylidene]pyrrolidine-1-carboxamide;
N-[(2Z)-5-methyl-3-(6-methylpyridazin-3-yl)-1,3-thiazol-2(3H)-ylidene]pyrrolidine-1-carboxamide;
N-[(2Z)-5-methyl-3-(6-methylpyrazin-2-yl)-1,3-thiazol-2(3H)-ylidene]pyrrolidine-1-carboxamide;
N-[(2Z)-5-methyl-3-pyrazin-2-yl-1,3-thiazol-2(3H)-ylidene]pyrrolidine-1-carboxamide;
N-[(2Z)-5-methyl-3-quinoxalin-2-yl-1,3-thiazol-2(3H)-ylidene]pyrrolidine-1-carboxamide;
N-[(2Z)-5-methyl-3-[5-(trifluoromethyl)pyridin-2-yl]-1,3-thiazol-2(3H)-ylidene]pyrrolidine-1-carboxamide;
N-[(2Z)-5-methyl-3-[6-(trifluoromethyl)pyridin-2-yl]-1,3-thiazol-2(3H)-ylidene]pyrrolidine-1-carboxamide;
N-[(2Z)-5-methyl-3-[4-(trifluoromethyl)pyridin-2-yl]-1,3-thiazol-2(3H)-ylidene]pyrrolidine-1-carboxamide;
N-[(2Z)-3-(4′-fluoro-1,1′-biphenyl-4-yl)-5-methyl-1,3-thiazol-2(3H)-ylidene]pyrrolidine-1-carboxamide;
N-[(2Z)-3-(4-acetylphenyl)-1,3-thiazol-2(3H)-ylidene]pyrrolidine-1-carboxamide;
N-[(2Z)-3-(4-propionylphenyl)-1,3-thiazol-2(3H)-ylidene]pyrrolidine-1-carboxamide;
N-[(2Z)-3-(4-tert-butylphenyl)-1,3-thiazol-2(3H)-ylidene]pyrrolidine-1-carboxamide;
N-[(2Z)-3-(4-ethylphenyl)-1,3-thiazol-2(3H)-ylidene]pyrrolidine-1-carboxamide;
N-[(2Z)-3-(2,4-dichlorophenyl)-1,3-thiazol-2(3H)-ylidene]pyrrolidine-1-carboxamide;
N-[(2Z)-3-(2-methoxyphenyl)-1,3-thiazol-2(3H)-ylidene]pyrrolidine-1-carboxamide;
N-[(2Z)-3-(2-methylphenyl)-1,3-thiazol-2(3H)-ylidene]pyrrolidine-1-carboxamide;
N-[(2Z)-3-(3,5-dichlorophenyl)-1,3-thiazol-2(3H)-ylidene]pyrrolidine-1-carboxamide;
N-[(2Z)-3-(3-methoxyphenyl)-1,3-thiazol-2(3H)-ylidene]pyrrolidine-1-carboxamide;
N-[(2Z)-3-(3-methylphenyl)-1,3-thiazol-2(3H)-ylidene]pyrrolidine-1-carboxamide;
N-[(2Z)-3-(4-methoxyphenyl)-1,3-thiazol-2(3H)-ylidene]pyrrolidine-1-carboxamide;
N-[(2Z)-3-(4-propylphenyl)-1,3-thiazol-2(3H)-ylidene]pyrrolidine-1-carboxamide;
N-[(2Z)-3-(2,4-dimethylphenyl)-1,3-thiazol-2(3H)-ylidene]pyrrolidine-1-carboxamide;
N-[(2Z)-3-pyridin-3-yl-1,3-thiazol-2(3H)-ylidene]pyrrolidine-1-carboxamide;
N-[(2Z)-3-(4-butylphenyl)-1,3-thiazol-2(3H)-ylidene]pyrrolidine-1-carboxamide;
N-[(2Z)-3-[3-(methylthio)phenyl]-1,3-thiazol-2(3H)-ylidene]pyrrolidine-1-carboxamide;
N-[(2Z)-3-(2,3-dichlorophenyl)-1,3-thiazol-2(3H)-ylidene]pyrrolidine-1-carboxamide;
N-[(2Z)-3-(2-chlorophenyl)-1,3-thiazol-2(3H)-ylidene]pyrrolidine-1-carboxamide;
N-[(2Z)-3-[2,3,5,6-tetrafluoro-4-(trifluoromethyl)phenyl]-1,3-thiazol-2(3H)-ylidene]pyrrolidine-1-carboxamide;
N-[(2Z)-3-(3,5-dimethylphenyl)-1,3-thiazol-2(3H)-ylidene]pyrrolidine-1-carboxamide;
N-[(2Z)-3-(3-isopropoxyphenyl)-1,3-thiazol-2(3H)-ylidene]pyrrolidine-1-carboxamide;
N-[(2Z)-3-[2,4-bis(trifluoromethyl)phenyl]-1,3-thiazol-2(3H)-ylidene]pyrrolidine-1-carboxamide;
N-[(2Z)-3-(3-cyanophenyl)-1,3-thiazol-2(3H)-ylidene]pyrrolidine-1-carboxamide;
N-[(2Z)-3-(1-oxo-2,3-dihydro-1H-inden-5-yl)-1,3-thiazol-2(3H)-ylidene]pyrrolidine-1-carboxamide;
N-[(2Z)-3-(4-methoxy-3-methylphenyl)-1,3-thiazol-2(3H)-ylidene]pyrrolidine-1-carboxamide;
N-[(2Z)-3-[4-(dimethylamino)phenyl]-5-methyl-1,3-thiazol-2(3H)-ylidene]-2,2-dimethylpropanamide;
2,2-dimethyl-N-[(2Z)-5-methyl-3-[4-(methylthio)phenyl]-1,3-thiazol-2(3H)-ylidene]propanamide;
N-[(2Z)-3-(4-acetylphenyl)-5-methyl-1,3-thiazol-2(3H)-ylidene]-2,2-dimethylpropanamide;
N-[(2Z)-3-(4-tert-butylphenyl)-5-methyl-1,3-thiazol-2(3H)-ylidene]-2,2-dimethylpropanamide;
N-[(2Z)-3-(4-ethylphenyl)-5-methyl-1,3-thiazol-2(3H)-ylidene]-2,2-dimethylpropanamide;
2,2-dimethyl-N-[(2Z)-5-methyl-3-(2-methylphenyl)-1,3-thiazol-2(3H)-ylidene]propanamide;
N-[(2Z)-3-[3-(dimethylamino)phenyl]-5-methyl-1,3-thiazol-2(3H)-ylidene]-2,2-dimethylpropanamide;
2,2-dimethyl-N-[(2Z)-5-methyl-3-phenyl-1,3-thiazol-2(3H)-ylidene]propanamide;
N-[(2Z)-3-(2-methoxyphenyl)-5-methyl-1,3-thiazol-2(3H)-ylidene]-2,2-dimethylpropanamide;
N-[(2Z)-3-(3-methoxyphenyl)-5-methyl-1,3-thiazol-2(3H)-ylidene]-2,2-dimethylpropanamide;
2,2-dimethyl-N-[(2Z)-5-methyl-3-(3-methylphenyl)-1,3-thiazol-2(3H)-ylidene]propanamide;
N-[(2Z)-3-(4-methoxyphenyl)-5-methyl-1,3-thiazol-2(3H)-ylidene]-2,2-dimethylpropanamide;
2,2-dimethyl-N-[(2Z)-5-methyl-3-(4-methylphenyl)-1,3-thiazol-2(3H)-ylidene]propanamide;
2,2-dimethyl-N-[(2Z)-5-methyl-3-(2-naphthyl)-1,3-thiazol-2(3H)-ylidene]propanamide;
N-[(2Z)-3-(6-methoxy-2-naphthyl)-5-methyl-1,3-thiazol-2(3H)-ylidene]-2,2-dimethylpropanamide;
N-[(2Z)-3-(2,4-dimethylphenyl)-5-methyl-1,3-thiazol-2(3H)-ylidene]-2,2-dimethylpropanamide;
N-[(2Z)-3-(4-chloro-3-methylphenyl)-5-methyl-1,3-thiazol-2(3H)-ylidene]-2,2-dimethylpropanamide;
N-[(2Z)-3-(4-butylphenyl)-5-methyl-1,3-thiazol-2(3H)-ylidene]-2,2-dimethylpropanamide;
N-[(2Z)-3-(3,4-dimethylphenyl)-5-methyl-1,3-thiazol-2(3H)-ylidene]-2,2-dimethylpropanamide;
2,2-dimethyl-N-[(2Z)-5-methyl-3-[3-(methylthio)phenyl]-1,3-thiazol-2(3H)-ylidene]propanamide;
N-[(2Z)-3-(3-chloro-4-methylphenyl)-5-methyl-1,3-thiazol-2(3H)-ylidene]-2,2-dimethylpropanamide;
N-[(2Z)-3-(3,5-dimethylphenyl)-5-methyl-1,3-thiazol-2(3H)-ylidene]-2,2-dimethylpropanamide;
N-[(2Z)-3-(3-isopropoxyphenyl)-5-methyl-1,3-thiazol-2(3H)-ylidene]-2,2-dimethylpropanamide;
N-[(2Z)-3-(4-chloro-3-methoxyphenyl)-5-methyl-1,3-thiazol-2(3H)-ylidene]-2,2-dimethylpropanamide;
N-[(2Z)-3-(4-methoxy-3-methylphenyl)-5-methyl-1,3-thiazol-2(3H)-ylidene]-2,2-dimethylpropanamide;
N-[(2Z)-3-(2-chlorophenyl)-5-methyl-1,3-thiazol-2(3H)-ylidene]-2,2-dimethylpropanamide;
2,2-dimethyl-N-[(2Z)-5-methyl-3-[2,3,5,6-tetrafluoro-4-(trifluoromethyl)phenyl]-1,3-thiazol-2(3H)-ylidene]propanamide;
2,2-dimethyl-N-[(2Z)-5-methyl-3-pyridin-3-yl-1,3-thiazol-2(3H)-ylidene]propanamide;
N-[(2Z)-3-(4-cyanophenyl)-5-methyl-1,3-thiazol-2(3H)-ylidene]-2,2-dimethylpropanamide;
N-[(2Z)-3-(3-cyanophenyl)-5-methyl-1,3-thiazol-2(3H)-ylidene]-2,2-dimethylpropanamide;
N-[(2Z)-3-(2′-methyl-1,1′-biphenyl-4-yl)-1,3-thiazol-2(3H)-ylidene]pyrrolidine-1-carboxamide;
N-[(2Z)-3-(3′-methyl-1,1′-biphenyl-4-yl)-1,3-thiazol-2(3H)-ylidene]pyrrolidine-1-carboxamide;
N-[(2Z)-3-(4′-methyl-1,1′-biphenyl-4-yl)-1,3-thiazol-2(3H)-ylidene]pyrrolidine-1-carboxamide;
N-[(2Z)-3-(3′-chloro-1,1′-biphenyl-4-yl)-1,3-thiazol-2(3H)-ylidene]pyrrolidine-1-carboxamide;
N-[(2Z)-3-(4′-chloro-1,1′-biphenyl-4-yl)-1,3-thiazol-2(3H)-ylidene]pyrrolidine-1-carboxamide;
N-[(2Z)-3-(4′-methoxy-1,1′-biphenyl-4-yl)-1,3-thiazol-2(3H)-ylidene]pyrrolidine-1-carboxamide;
N-[(2Z)-3-[3′-(trifluoromethyl)-1,1′-biphenyl-4-yl]-1,3-thiazol-2(3H)-ylidene]pyrrolidine-1-carboxamide;
N-[(2Z)-3-(4-thien-3-ylphenyl)-1,3-thiazol-2(3H)-ylidene]pyrrolidine-1-carboxamide;
N-[(2Z)-3-[4′-(trifluoromethyl)-1,1′-biphenyl-4-yl]-1,3-thiazol-2(3H)-ylidene]pyrrolidine-1-carboxamide;
N-[(2Z)-3-(3′-ethoxy-1,1′-biphenyl-4-yl)-1,3-thiazol-2(3H)-ylidene]pyrrolidine-1-carboxamide;
N-[(2Z)-3-(3′-methoxy-1,1′-biphenyl-4-yl)-1,3-thiazol-2(3H)-ylidene]pyrrolidine-1-carboxamide;
N-[(2Z)-3-(3′,5′-dichloro-1,1′-biphenyl-4-yl)-1,3-thiazol-2(3H)-ylidene]pyrrolidine-1-carboxamide;
N-[(2Z)-3-(2′-chloro-1,1′-biphenyl-4-yl)-1,3-thiazol-2(3H)-ylidene]pyrrolidine-1-carboxamide;
N-[(2Z)-3-(2′-methoxy-1,1′-biphenyl-4-yl)-1,3-thiazol-2(3H)-ylidene]pyrrolidine-1-carboxamide;
N-[(2Z)-3-(2′-acetyl-1,1′-biphenyl-4-yl)-1,3-thiazol-2(3H)-ylidene]pyrrolidine-1-carboxamide;
N-[(2Z)-3-(3′-acetyl-1,1′-biphenyl-4-yl)-1,3-thiazol-2(3H)-ylidene]pyrrolidine-1-carboxamide;
N-[(2Z)-3-(4′-acetyl-1,1′-biphenyl-4-yl)-1,3-thiazol-2(3H)-ylidene]pyrrolidine-1-carboxamide;
N-[(2Z)-3-(3′,4′-dichloro-1,1′-biphenyl-4-yl)-1,3-thiazol-2(3H)-ylidene]pyrrolidine-1-carboxamide;
N-[(2Z)-3-(4-pyridin-3-ylphenyl)-1,3-thiazol-2(3H)-ylidene]pyrrolidine-1-carboxamide;
N-[(2Z)-3-(2′,5′-dichloro-1,1′-biphenyl-4-yl)-1,3-thiazol-2(3H)-ylidene]pyrrolidine-1-carboxamide;
N-[(2Z)-3-(3′-cyano-1,1′-biphenyl-4-yl)-1,3-thiazol-2(3H)-ylidene]pyrrolidine-1-carboxamide;
N-[(2Z)-3-(2′,3′-dichloro-1,1′-biphenyl-4-yl)-1,3-thiazol-2(3H)-ylidene]pyrrolidine-1-carboxamide;
N-[(2Z)-3-(2′,4′-dichloro-1,1′-biphenyl-4-yl)-1,3-thiazol-2(3H)-ylidene]pyrrolidine-1-carboxamide;
N-[(2Z)-3-(4′-cyano-1,1′-biphenyl-4-yl)-1,3-thiazol-2(3H)-ylidene]pyrrolidine-1-carboxamide;
N-[(2Z)-3-[4-(1-methyl-1H-pyrazol-4-yl)phenyl]-1,3-thiazol-2(3H)-ylidene]pyrrolidine-1-carboxamide;
N-[(2Z)-3-[4-(6-methoxypyridin-3-yl)phenyl]-1,3-thiazol-2(3H)-ylidene]pyrrolidine-1-carboxamide;
N-[(2Z)-3-(1,1′-biphenyl-4-yl)-1,3-thiazol-2(3H)-ylidene]pyrrolidine-1-carboxamide;
N-[(2Z)-3-[4-(2-methoxypyrimidin-5-yl)phenyl]-1,3-thiazol-2(3H)-ylidene]pyrrolidine-1-carboxamide;
N-[(2Z)-3-[4′-(cyanomethyl)-1,1′-biphenyl-4-yl]-1,3-thiazol-2(3H)-ylidene]pyrrolidine-1-carboxamide;
N-[(2Z)-3-quinoxalin-6-yl-1,3-thiazol-2(3H)-ylidene]pyrrolidine-1-carboxamide;
N-[(2Z)-3-(1-benzothien-5-yl)-1,3-thiazol-2(3H)-ylidene]pyrrolidine-1-carboxamide;
N-[(2Z)-3-pyrazin-2-yl-1,3-thiazol-2(3H)-ylidene]pyrrolidine-1-carboxamide;
N-[(2Z)-3-quinoxalin-2-yl-1,3-thiazol-2(3H)-ylidene]pyrrolidine-1-carboxamide;
N-[(2Z)-3-[2-(methylthio)pyrimidin-4-yl]-1,3-thiazol-2(3H)-ylidene]pyrrolidine-1-carboxamide;
N-[(2Z)-3-(6-phenylpyridazin-3-yl)-1,3-thiazol-2(3H)-ylidene]pyrrolidine-1-carboxamide;
N-[(2Z)-3-(6-methylpyridin-2-yl)-1,3-thiazol-2(3H)-ylidene]pyrrolidine-1-carboxamide;
N-[(2Z)-3-(1-benzothien-3-yl)-1,3-thiazol-2(3H)-ylidene]pyrrolidine-1-carboxamide;
N-[(2Z)-3-(4′-fluoro-1,1′-biphenyl-4-yl)-1,3-thiazol-2(3H)-ylidene]pyrrolidine-1-carboxamide;
N-[(2Z)-3-(5-fluoropyridin-2-yl)-1,3-thiazol-2(3H)-ylidene]pyrrolidine-1-carboxamide;
N-[(2Z)-3-(6-fluoropyridin-3-yl)-1,3-thiazol-2(3H)-ylidene]pyrrolidine-1-carboxamide;
N-[(2Z)-3-[5-(trifluoromethyl)pyridin-3-yl]-1,3-thiazol-2(3H)-ylidene]pyrrolidine-1-carboxamide;
N-[(2Z)-3-[5-(trifluoromethyl)pyridin-2-yl]-1,3-thiazol-2(3H)-ylidene]pyrrolidine-1-carboxamide;
N-[(2Z)-3-[4-(trifluoromethyl)pyridin-2-yl]-1,3-thiazol-2(3H)-ylidene]pyrrolidine-1-carboxamide;
N-[(2Z)-3-[6-(trifluoromethyl)pyridin-2-yl]-1,3-thiazol-2(3H)-ylidene]pyrrolidine-1-carboxamide;
N-[(2Z)-3-(6-ethoxy-2-naphthyl)-1,3-thiazol-2(3H)-ylidene]pyrrolidine-1-carboxamide;
N-[(2Z)-3-pyrido[2,3-b]pyrazin-7-yl-1,3-thiazol-2(3H)-ylidene]pyrrolidine-1-carboxamide;
N-[(2Z)-3-(4-cyclohexylphenyl)-1,3-thiazol-2(3H)-ylidene]pyrrolidine-1-carboxamide;
N-[(2Z)-3-(5-chloropyridin-2-yl)-1,3-thiazol-2(3H)-ylidene]pyrrolidine-1-carboxamide;
N-[(2Z)-3-[2-cyano-4-(trifluoromethyl)phenyl]-1,3-thiazol-2(3H)-ylidene]pyrrolidine-1-carboxamide;
N-[(2Z)-3-(5-phenylthien-2-yl)-1,3-thiazol-2(3H)-ylidene]pyrrolidine-1-carboxamide;
N-[(2Z)-3-[6-(1H-pyrazol-1-yl)pyridin-2-yl]-1,3-thiazol-2(3H)-ylidene]pyrrolidine-1-carboxamide;
N-[(2Z)-3-(6-methylpyrazin-2-yl)-1,3-thiazol-2(3H)-ylidene]pyrrolidine-1-carboxamide;
N-[(2Z)-3-(2-methylpyrimidin-4-yl)-1,3-thiazol-2(3H)-ylidene]pyrrolidine-1-carboxamide;
N-[(2Z)-3-(2,6-dimethylpyrimidin-4-yl)-1,3-thiazol-2(3H)-ylidene]pyrrolidine-1-carboxamide;
N-[(2Z)-3-(2-fluoropyridin-4-yl)-1,3-thiazol-2(3H)-ylidene]pyrrolidine-1-carboxamide;
N-[(2Z)-3-(2-methyl-1,3-benzothiazol-5-yl)-1,3-thiazol-2(3H)-ylidene]pyrrolidine-1-carboxamide;
(3R)-3-fluoro-N-[(2Z)-3-(4-isopropylphenyl)-5-methyl-1,3-thiazol-2(3H)-ylidene]pyrrolidine-1-carboxamide;
(3R)—N-[(2Z)-3-(3,4-dimethylphenyl)-5-methyl-1,3-thiazol-2(3H)-ylidene]-3-fluoropyrrolidine-1-carboxamide;
(3R)-3-fluoro-N-[(2Z)-3-(4-methoxyphenyl)-5-methyl-1,3-thiazol-2(3H)-ylidene]pyrrolidine-1-carboxamide;
(3R)-3-fluoro-N-[(2Z)-3-(4′-fluoro-1,1′-biphenyl-4-yl)-5-methyl-1,3-thiazol-2(3H)-ylidene]pyrrolidine-1-carboxamide;
2,2-dimethyl-N-[(2Z)-5-methyl-3-quinoxalin-6-yl-1,3-thiazol-2(3H)-ylidene]propanamide;
N-[(2Z)-3-(1-benzothien-5-yl)-5-methyl-1,3-thiazol-2(3H)-ylidene]-2,2-dimethylpropanamide;
2,2-dimethyl-N-[(2Z)-5-methyl-3-pyrazin-2-yl-1,3-thiazol-2(3H)-ylidene]propanamide;
2,2-dimethyl-N-[(2Z)-5-methyl-3-quinoxalin-2-yl-1,3-thiazol-2(3H)-ylidene]propanamide;
2,2-dimethyl-N-[(2Z)-5-methyl-3-[2-(methylthio)pyrimidin-4-yl]-1,3-thiazol-2(3H)-ylidene]propanamide;
2,2-dimethyl-N-[(2Z)-5-methyl-3-(6-phenylpyridazin-3-yl)-1,3-thiazol-2(3H)-ylidene]propanamide;
2,2-dimethyl-N-[(2Z)-5-methyl-3-(6-methylpyridin-2-yl)-1,3-thiazol-2(3H)-ylidene]propanamide;
N-[(2Z)-3-(1-benzothien-3-yl)-5-methyl-1,3-thiazol-2(3H)-ylidene]-2,2-dimethylpropanamide;
N-[(2Z)-3-(4′-fluoro-1,1′-biphenyl-4-yl)-5-methyl-1,3-thiazol-2(3H)-ylidene]-2,2-dimethylpropanamide;
N-[(2Z)-3-(5-fluoropyridin-2-yl)-5-methyl-1,3-thiazol-2(3H)-ylidene]-2,2-dimethylpropanamide;
N-[(2Z)-3-(6-chloroquinoxalin-2-yl)-5-methyl-1,3-thiazol-2(3H)-ylidene]-2,2-dimethylpropanamide;
2,2-dimethyl-N-[(2Z)-5-methyl-3-[5-(trifluoromethyl)pyridin-3-yl]-1,3-thiazol-2(3H)-ylidene]propanamide;
2,2-dimethyl-N-[(2Z)-5-methyl-3-[5-(trifluoromethyl)pyridin-2-yl]-1,3-thiazol-2(3H)-ylidene]propanamide;
2,2-dimethyl-N-[(2Z)-5-methyl-3-[4-(trifluoromethyl)pyridin-2-yl]-1,3-thiazol-2(3H)-ylidene]propanamide;
2,2-dimethyl-N-[(2Z)-5-methyl-3-[6-(trifluoromethyl)pyridin-2-yl]-1,3-thiazol-2(3H)-ylidene]propanamide;
2,2-dimethyl-N-[(2Z)-5-methyl-3-(6-methylpyridazin-3-yl)-1,3-thiazol-2(3H)-ylidene]propanamide;
2,2-dimethyl-N-[(2Z)-5-methyl-3-pyrido[2,3-b]pyrazin-7-yl-1,3-thiazol-2(3H)-ylidene]propanamide;
N-[(2Z)-3-(4-cyclohexylphenyl)-5-methyl-1,3-thiazol-2(3H)-ylidene]-2,2-dimethylpropanamide;
N-[(2Z)-3-(5-chloropyridin-2-yl)-5-methyl-1,3-thiazol-2(3H)-ylidene]-2,2-dimethylpropanamide;
N-[(2Z)-3-[2-cyano-4-(trifluoromethyl)phenyl]-5-methyl-1,3-thiazol-2(3H)-ylidene]-2,2-dimethylpropanamide;
2,2-dimethyl-N-[(2Z)-5-methyl-3-[2-(trifluoromethyl)pyrimidin-4-yl]-1,3-thiazol-2(3H)-ylidene]propanamide;
2,2-dimethyl-N-[(2Z)-5-methyl-3-(5-phenylthien-2-yl)-1,3-thiazol-2(3H)-ylidene]propanamide;
2,2-dimethyl-N-[(2Z)-5-methyl-3-(6-methylpyrazin-2-yl)-1,3-thiazol-2(3H)-ylidene]propanamide; and
2,2-dimethyl-N-[(2Z)-5-methyl-3-(2-methylpyrimidin-4-yl)-1,3-thiazol-2(3H)-ylidene]propanamide;
or a pharmaceutically acceptable salt, ester, amide or prodrug thereof.

11. The composition of claim 1, wherein the nicotinic receptor ligand is N-[(3R)-1-azabicyclo[2.2.2]oct-3-yl]-4-chlorobenzamide (PNU-282987), MEM-3454, ARR-17779, AZD0328, WB-56203, SSR-180711A, GTS21, OH-GTS-21, XY4083, TC-5619, isoprinicline, or varenicline.

12. The composition of claim 1, wherein the histamine-3 receptor ligand is selected from aminoalkoxybiphenyl carboxamide derivatives, benzofuranyl amine derivatives, bicyclic substituted amine derivatives, di- and tri-substituted pyrrolidine derivatives, octahydropyrrolopyrrole derivatives, phenylcyclobutane derivatives, benzothiazole derivatives, azacyclosteroid derivatives, and cyclopropyl amine derivatives.

13. The composition of claim 1, wherein the histamine-3 receptor ligand is selected from:
[4-(2-{2-[(2R)-2-methylpyrrolidinyl]ethyl}-benzofuran-5-yl)benzonitrile;
or is a pharmaceutically acceptable salt thereof.

14. The composition of claim 1, wherein the histamine-3 receptor ligand is selected from ciproxifan; 6-[(3-cyclobutyl-2,3,4,5-tetrahydro-1H-3-benzazepin-7-yl)oxy]-N-methyl-3-pyridinecarboxamide hydrochloride; GSK-239512; (1-{6-[(3-cyclobutyl-2,3,4,5-tetrahydro-1H-3-benzaaepin-7-yl)oxy]-3-pyridinyl}-2-pyrrolidinone; MK-0249; 3-(4-[1-cyclobutyl-4-piperidinyl)oxy]phenyl)-2-methyl-5-(trifluoromethyl)-4(3H)-quinazolinone, 2-methyl-3-(4-(3-(pyrrolidin-1-yl)propoxy)phenyl)-5-(trifluoromethyl)quinazolin-4(3H)-one; BF-2649; (1-{3-[3-(4-chlorophenyl)propoxy]propyl}piperidine); JNJ-17216498; (4-isopropylpiperazin-1-yl)(4-(piperidin-1-ylmethyl)phenyl)methanone; (4-isopropylpiperazin-1-yl)(4-(morpholinomethyl)phenyl)methanone; PFE-03654746; 6-(2-(1-isopropylpiperidin-4-yloxy)-7,8-dihydro-1,6-naphthyridin-6(5H)-yl)nicotinamide; (1 r,3r)-N-ethyl-3-fluoro-3-(4-(3-fluoro-4-(pyrrolidin-1-ylmethyl)phenyl)cyclohexyl)cyclobutanecarboxamide; N-ethyl-3-fluoro-3-[3-fluoro-4-(pyrrolidin-1-ylmethyl)phenyl]cyclobutanecarboxamide; SCH-497079; (1-((2-amino-6-methylpyridin-4-yl)methyl)-4-fluoropiperidin-4-yl)(4-(2-(pyridin-2-yl)-3H-imidazo[4,5-b]pyridin-3-yl)piperidin-1-yl)methanone; CEP-26401; 6-{4-[3-(r-2-methyl-pyrrolidin-1-yl)-propoxy]-phenyl}-2h-pyridazin-3-one; SAR-110894; APD916; and 4′-(2-(2-methylpyrrolidin-1-yl)ethyl)-N-(tetrahydro-2H-pyran-4-yl)biphenyl-4-sulfonamide.

15. The composition of claim 1, wherein the nicotinic receptor ligand is present in a sub efficacious amount for treating a cognitive disorder.

16. The composition of claim 1, wherein the histamine-3 receptor ligand is present in a sub efficacious amount for treating a cognitive disorder.

17. The composition of claim 1, wherein the nicotinic receptor ligand is present in a therapeutically effective amount and the histamine-3 receptor ligand is present in a therapeutically effective amount.

18. A method for treating a cognitive disorder in a patient, comprising:
(i) administering an amount of nicotinic receptor ligand to the patient; and
(ii) administering an amount of histamine-3 receptor ligand to the patient; wherein the amounts of (i) and (ii) together are effective in treating a cognitive disorder.

19. A composition, comprising:
(i) nicotinic subtype α4β2 receptor ligand or a histamine-3 receptor ligand; and
(ii) a psychostimulant, or a monoamine reuptake inhibitor.

20. The composition of claim 19, wherein the psychostimulant is selected from amphetamine, methylphenidate, and pemoline.

21. The composition of claim 19, wherein the monoamine reuptake inhibitor is desipramine, notriptyline, atomoxetine (or tomoxetine), reboxetine, venlafaxine, citalopram, escitalopram, fluoxetine, and bupropion.

22. The composition of claim 19, wherein the psychostimulant is modafinil or armodafinil.

23. A method for treating a cognitive disorder in a patient, comprising:
(i) administering an amount of a nicotinic subtype α4β2 receptor ligand or a histamine-3 receptor ligand to the patient; and
(ii) administering an amount of a psychostimulant, a monoamine reuptake inhibitor, or both a psychostimulant and a monoamine reuptake inhibitor, to the patient; wherein the amounts of (i) and (ii) together are effective in treating a cognitive disorder.

24. An article of manufacture, comprising:
(i) a first pharmaceutical dosage form comprising at least one nicotinic acetylcholine receptor ligand;
(ii) a second pharmaceutical dosage form comprising at least one histamine-3 receptor ligand;
wherein the article contains first and second pharmaceutical dosage forms.

25. An article of manufacture, comprising:
(i) a first pharmaceutical dosage form comprising at least one nicotinic subtype α4β2 receptor ligand or histamine-3 receptor ligand;
(ii) a second pharmaceutical dosage form comprising at least one psychostimulant, a monoamine reuptake inhibitor, or both a psychostimulant and a monoamine reuptake inhibitor, to the patient;
wherein the article contains first and second pharmaceutical dosage forms.
